(19) United States
(12) Patent Application Publication
(10) Pub. No.: US 2018/0346901 A1
(43) Pub. Date: Dec. 6, 2018

(54) MEANS AND METHODS FOR PREPARING ENGINEERED PROTEINS BY GENETIC CODE EXPANSION IN INSECT CELLS

(71) Applicant: EUROPEAN MOLECULAR BIOLOGY LABORATORY, Heidelberg (DE)

(72) Inventors: Imre BERGER, St Egreve (FR); Edward LEMKE, Mannheim (DE); Christine KOEHLER, Forst (DE); Gemma ESTRADA GIRONA, Heidelberg (DE)

(21) Appl. No.: 15/780,187

(22) PCT Filed: Nov. 29, 2016
(86) PCT No.: PCT/EP2016/079140

§ 371 Date: May 30, 2018

(30) Foreign Application Priority Data
Nov. 30, 2015 (EP) 15197057.1

(51) Int. Cl. C12N 9/00; C12N 15/67; C12N 15/52; C12N 15/86; C12N 5/07; C07K 16/18; C07K 16/32; C07K 14/47; C07K 16/24

(52) U.S. Cl. CPC C12N 9/93; C12N 15/67; C12N 15/52; C12N 15/86; C12N 5/0601; C07K 16/18; C07K 2317/55; C07K 16/32; C07K 14/47; C07K 16/248; C12N 2710/14143; C12N 2510/02; C12Y 601/01026

(57) ABSTRACT

The invention relates to a method of preparing engineered target polypeptides (TP) comprising in its amino acid sequence one or more, identical or different, non-canonical amino acid (ncAA) residues, by expressing said TP in an insect cell line (ICL) and by expressing novel orthogonal bacterial aminoacyl tRNA synthetase/tRNA pairs in said ICL; a baculoviral shuttle vector (bacmid) suitable or introducing the genetic information of said orthogonal tRNA synthetase/tRNA into said ILC; particular expression cassettes for expressing said particular tRNAs in said ILC; TPs obtained by said method; as we as a kit for preparing said TPs.

### FIELD OF THE INVENTION

The invention relates to a method of preparing engineered target polypeptides (TP) comprising in its amino acid sequence one or more, identical or different, non-canonical amino acid (ncAA) residues, by expressing said TP in an insect cell line (ICL) and by expressing novel orthogonal bacterial aminoacyl tRNA synthetase/tRNA pairs in said ICL; a baculoviral shuttle vector (bacmid) suitable for introducing the genetic information of said orthogonal tRNA synthetase/tRNA into said ILC; particular expression cassettes for expressing said particular tRNAs in said ILC; TPs obtained by said method; as well as a kit for preparing said TPs.

### BACKGROUND OF THE INVENTION

The incorporation of non-canonical amino acids (ncAAs) is a major tool for functionalization of proteins in E. coli and eukaryotic cells. Genetic code expansion is used since decades and is meanwhile well established in E. coli, as well as in eukaryotic system, like mammalians (Chatterjee et al, PNAS 2013, 110, 29: 11803-11808), yeast (Chin, J. W., Cropp, T. A., Anderson, J. C., Mukherji, M., Zhang, Z., and Schultz, P. G. (2003). Science 301, 964-967) or Drosophila melanogaster (Mukai, T. et al Protein Science 2010, 19: 440-448) The system is used to incorporate a non-canonical amino acid (ncAA) site-specifically into a protein. Introduction of non-canonical amino acids with various functional groups is applied e.g. in labeling of proteins for single molecule studies or super resolution microscopy, cross-linking of proteins or attaching a post-translational modification of choice. For this purpose, an synthetase/tRNA pair (which is orthogonal to the expression host) has to be co-transfected with the protein of interest. The synthetase can recognize the non-canonical amino acid, which will be inserted into the elongated protein chain, in response to the amber stop codon. Several systems already exist, e.g. Methanococcus jannaschii TyrRS/tRNA^{T yr} or Methanosarcina mazei PylRS/tRNA^{Pyl} (Liu, C. C., and Schultz, P. G. (2010). Annual review of biochemistry 79, 413-444.

The amber stop codon, UAG, has been successfully used in in vitro biosynthetic system and in Xenopus oocytes to direct the incorporation of unnatural amino acids. Among the three stop codons, UAG is the least used stop codon in E. coli. Some E. coli strains contain natural suppressor tRNAs, which recognize UAG and insert a natural amino acid. In addition, these amber suppressor tRNAs have been used in conventional protein mutagenesis.

In E. coli proteins of small and medium size can be easily expressed in large amounts. However, such simple laboratory hosts are not well suited for expression of large multi-protein complexes with ideally native eukaryotic posttranslational protein modifications. For the expression of high molecular weight proteins or protein complexes, especially originating from eukaryotic organisms, other expression hosts, e.g. mammalian cultures, are preferred. In addition to the size limit, also most posttranslational modifications are absent in E. coli expressed proteins.

Mukai et al (see above), developed a D. melanogaster Schneider 2-cell-based system for incorporating ncAAs into proteins at specific sites. Different expression systems comprising prokaryotic tRNA^{Tyr} were construed and examined in S2 cells. An expression system designated U6-EYR comprising E. coli tRNA^{Tyr} under the control of the D. melanogaster U6 promoter No.2 worked best. A plasmid vector carrying three copies of U6-EYR and the coding sequence of E. coli TyrRS specific for 3-iodo-L-tyrosine was used for stably transfecting S2 cells. Furthermore, analogously Chin et al have shown that the Methanosarcina pyrrolysine tRNA/RS pair can be used in Drosophila cells and animals (Bianco, A., Townsley, F. M., Greiss, S., Lang, K., and Chin, J. W. (2012). r. Nature chemical biology 8, 748-750. And Elliott, T. S., Townsley, F. M., Bianco, A., Ernst, R. J., Sachdeva, A., Elsasser, S. J., Davis, L., Lang, K., Pisa, R., Greiss, S., et al. (2014). Nature biotechnology 32, 465-472).

However, eukaryotic protein expression of engineered proteins as described in the above-mentioned prior art is still not satisfactory.

Thus, the problem to be solved by the invention was to develop novel methods and tools which allow in insect cell lines the cost-effective large scale expression of properly processed eukaryotic proteins or multiple proteins carrying non-canonical amino acid residues within their sequence so that post-translational modification of the expressed protein may be effected.

### SUMMARY OF THE INVENTION

The problem of the invention was surprisingly solved by establishing genetic code expansion in insect cell lines, in particular Sf21 cells, combined with a revised Baculovirus vector. In particular the inventors shuffled an orthogonal synthetase PylRS/tRNA pair into a widely used Bacmid vector, resulting in new DH10 Bac-TAG cells. In particular, the MultiBac system, which is a versatile platform to easily generate large protein assemblies and express them in eukaryotes is applied. By said method the inventors succeeded in introducing ncAA into green fluorescent protein (GFP), as well as in a number of different multi-protein complexes.

### BRIEF DESCRIPTION OF THE FIGURES

FIG. 1 shows snRNA U6 genes from various organism (Homo sapiens, Drosophila melanogaster, Spodoptera frugiperda and Bombyx mori) (SEQ ID NO: 10 to 14). The snRNA genes from Human and Drosophila were extracted from genBank, as well as snRNA U6 isoform E. For Spodoptera frugiperda and the first Bombyx mori snRNA shown, the sequence was taken out of the genome sequence. All the genes are highly similar to each other, only a minor proportion of nucleotides are different (highlighted by underlining). Also the sequences are equal in length, merely the snRNA U6 Bombyx mori isoform E has 15 nucleotides missing at the N-terminus (5′-end) and 13 nucleotides plus the polyT-tail at the C-terminus (3′-end).

FIG. 2 shows the alignments of snRNA U6 genes (highlighted by underlining) and corresponding upstream U6 promoter regions and corresponding downstream 3′termination signals of Spodoptera frugiperda, aligned to U6 promoter, snRNA U6 and 3′termination of Bombyx mori. The alignment, which was done using ClustalW (Larkin, M. A. et al. Clustal W and Clustal X version 2.0. Bioinformatics 23, 2947-2948 (2007). Goujon, M. et al. A new bioinformatics analysis tools framework at EMBL-EBI. Nucleic acids research 38, W695-699 (2010)) shows that the snRNA U6 gene is well conserved. The promoter regions look similar, especially at the region close to the snRNA gene, same is true for the 3′ termination signal. Only the U6-1 and U6-3 sequences show a higher degree of misalignments compared to the other ones.

FIG. 3 shows different tRNA^{Pyl} constructs and the FACS results of Sf21 cells transfected with these constructs.

A: The U6 promoter sequences of different organism (Human, Drosophila, Bombyx and Spodoptera) are illustrated, upstream of the tRNA^{Pyl} gene from Methanosarcina mazei, followed by the corresponding downstream 3′termination signal of each snRNA U6 gene.

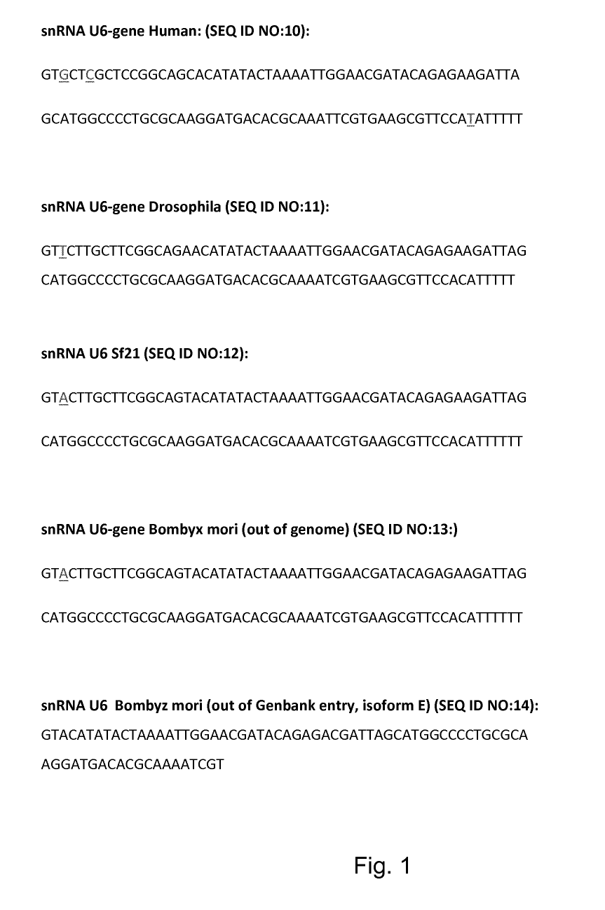

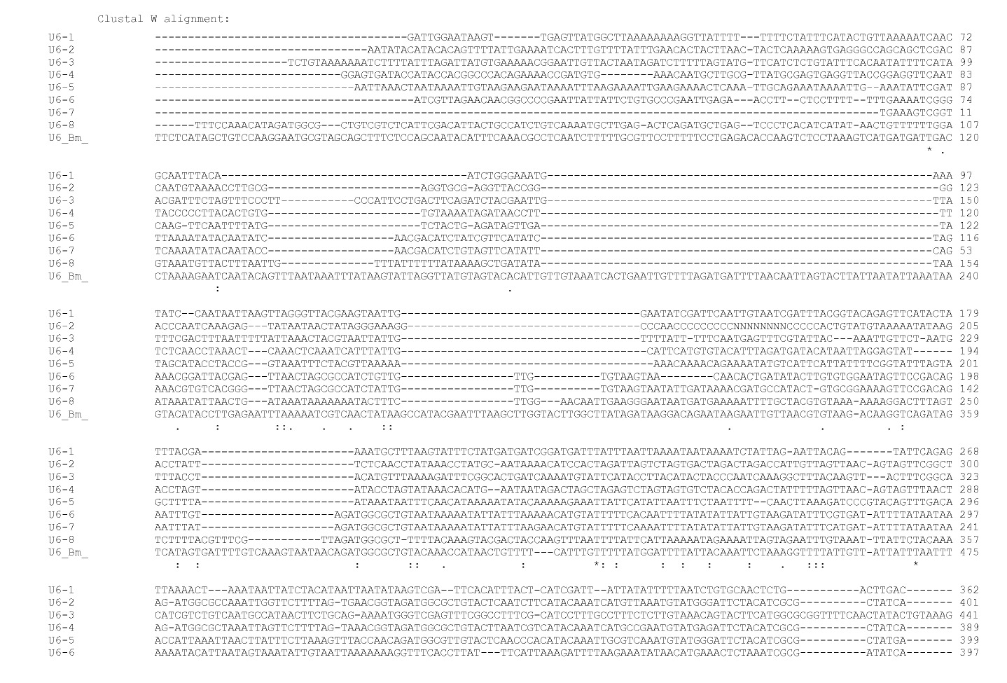

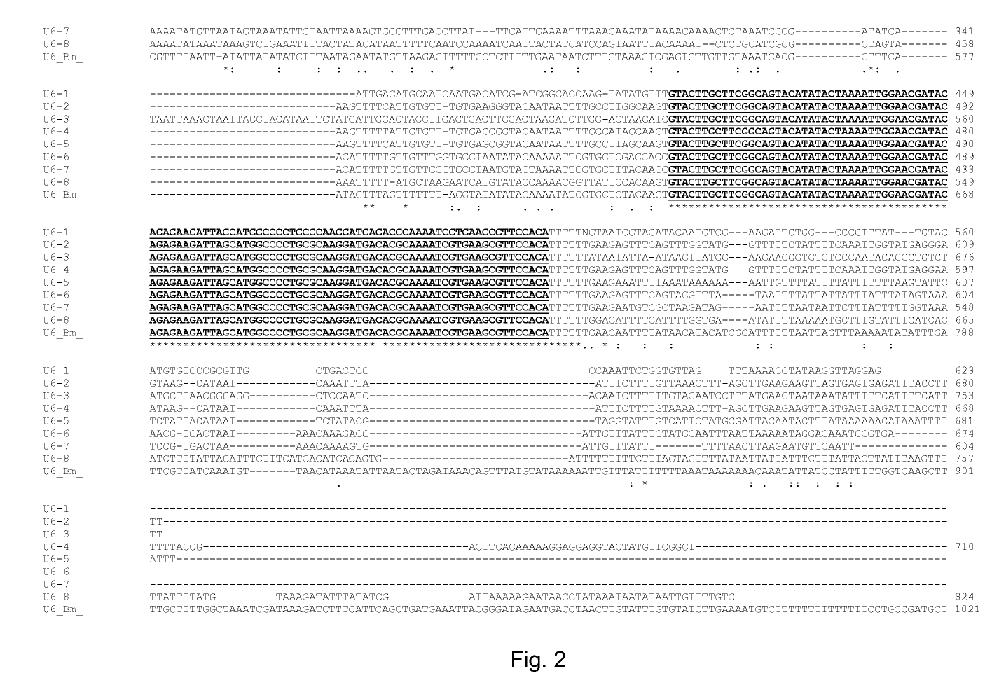

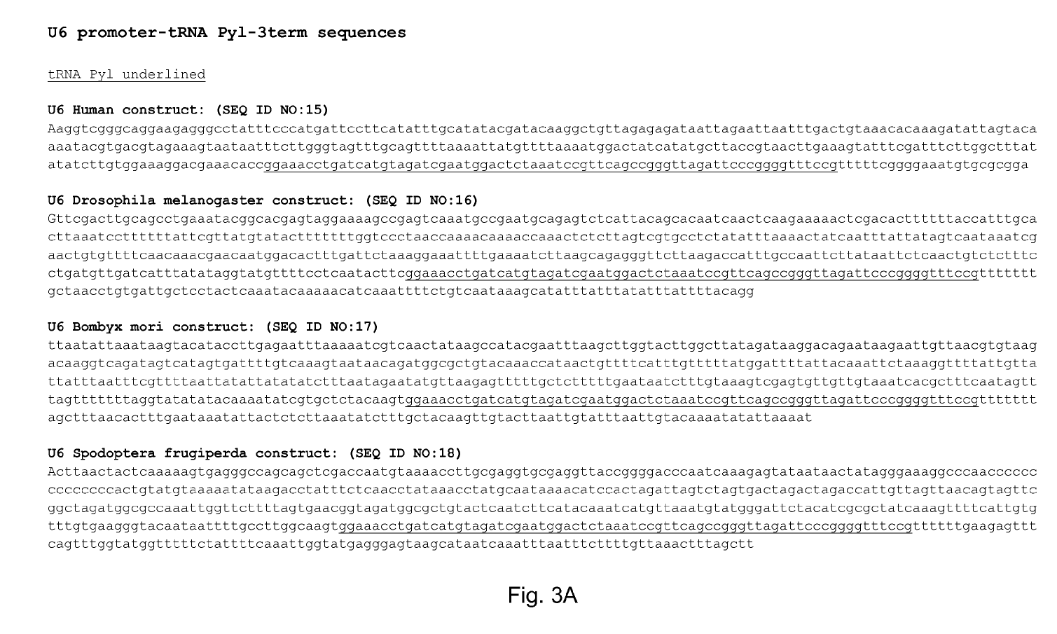

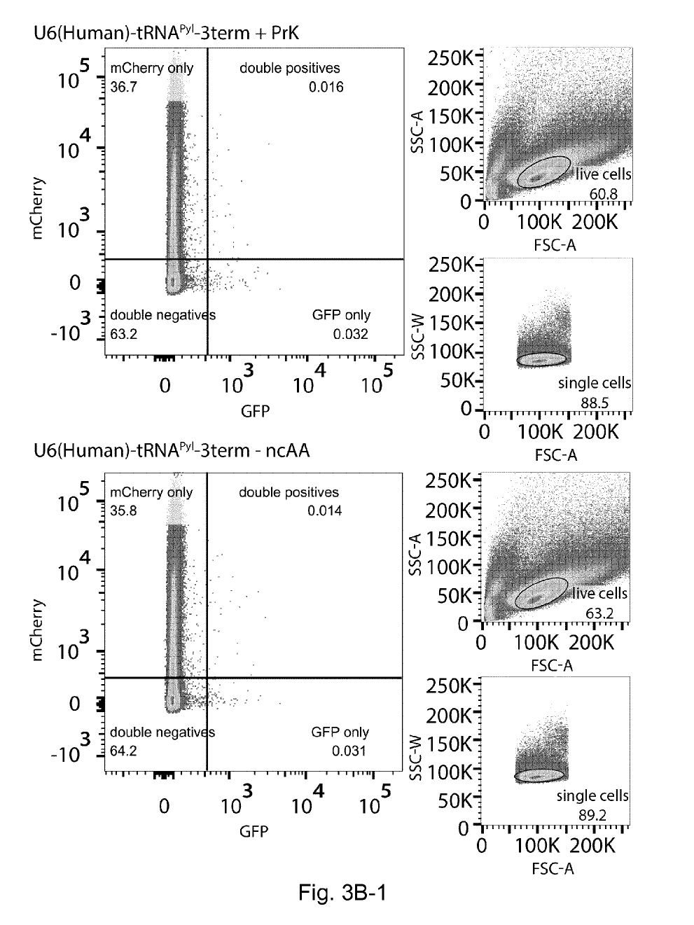

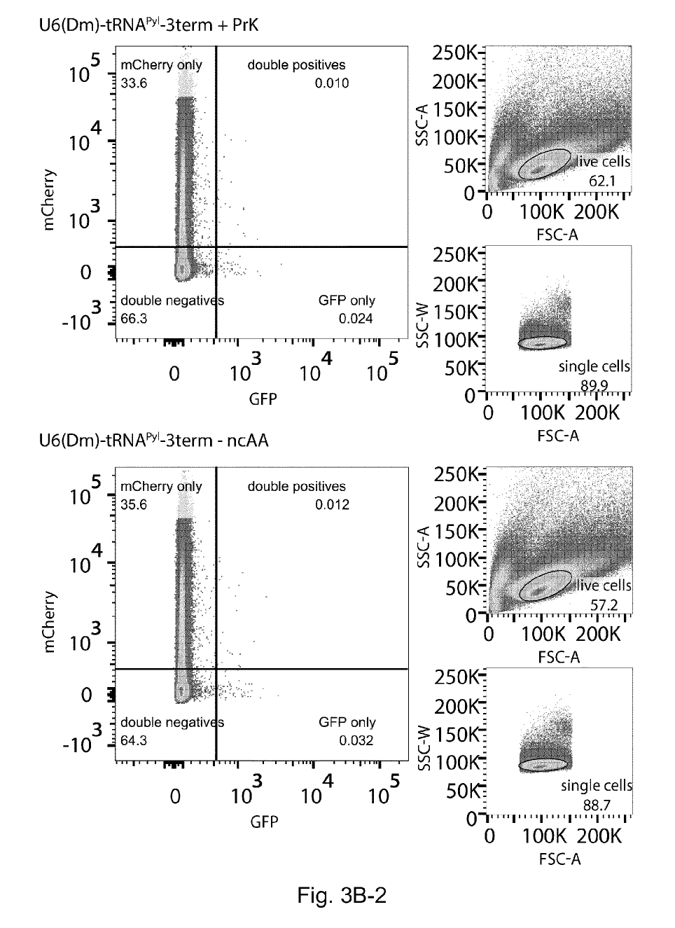

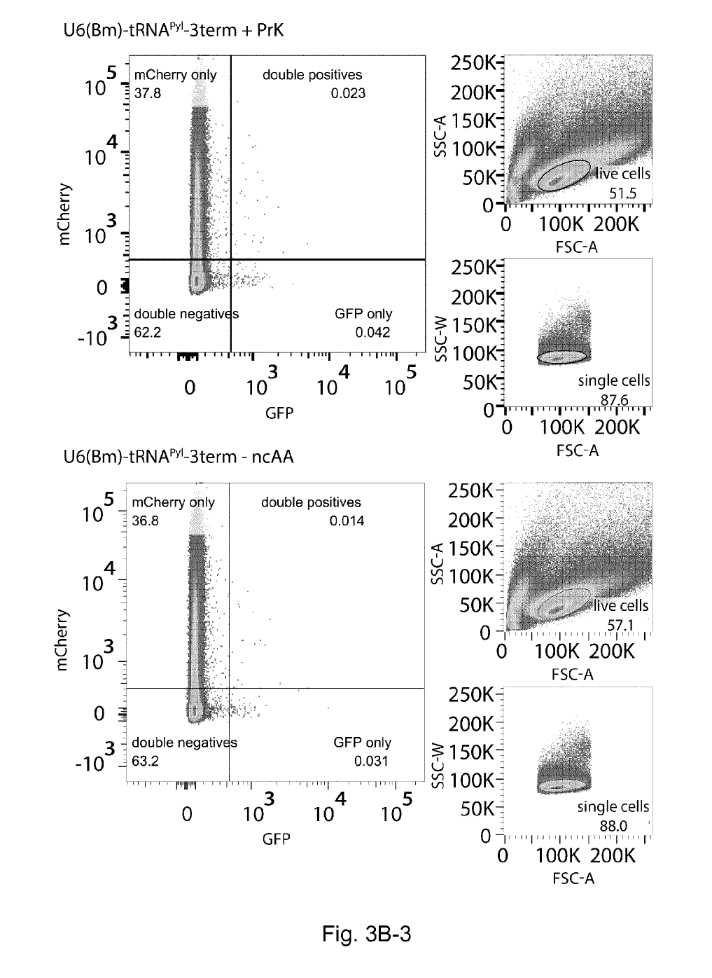

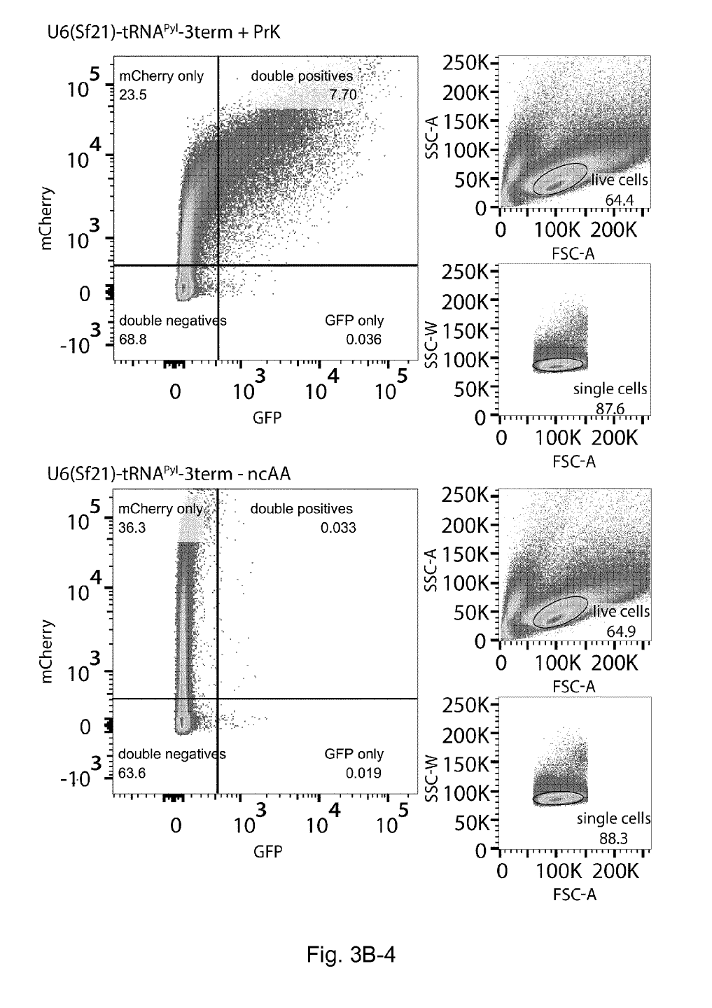

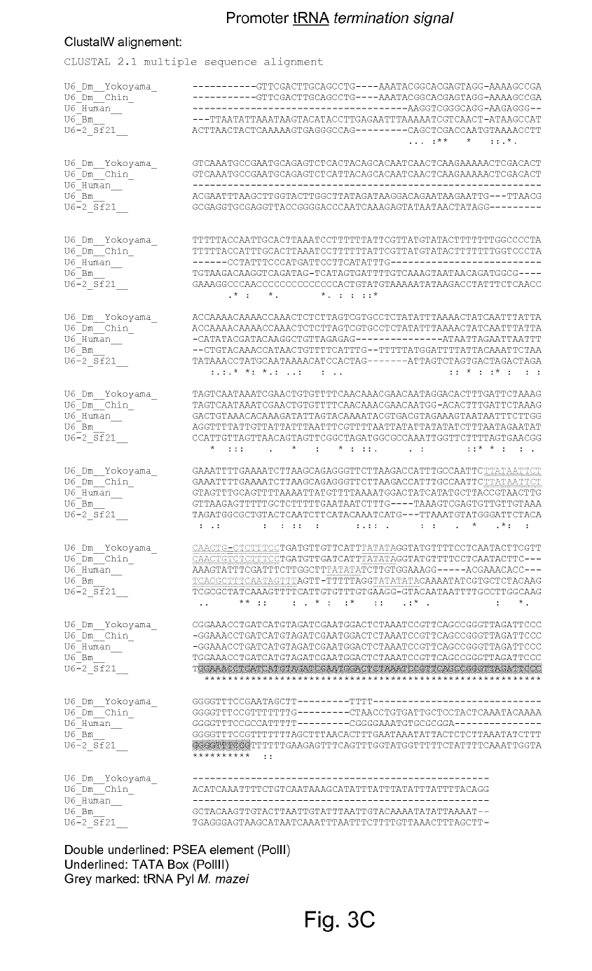

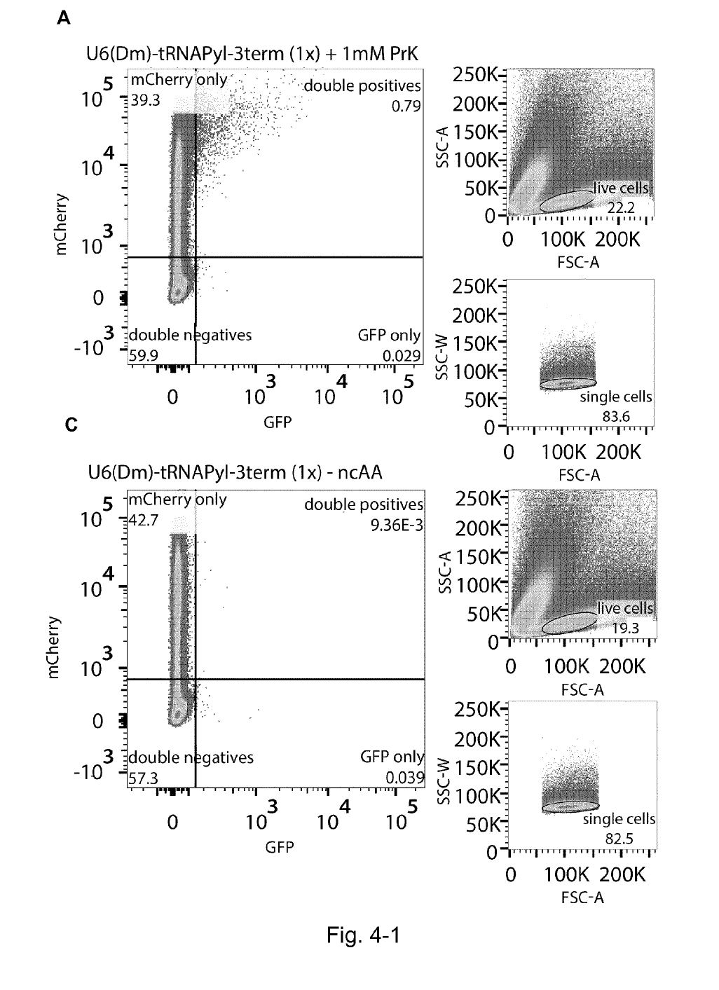

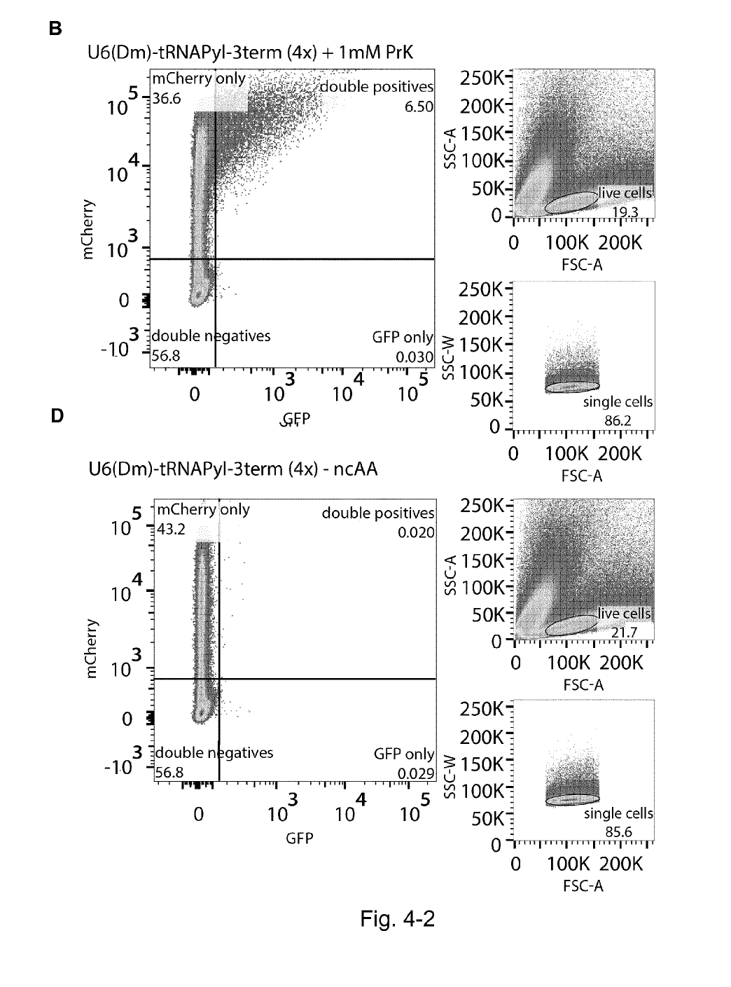

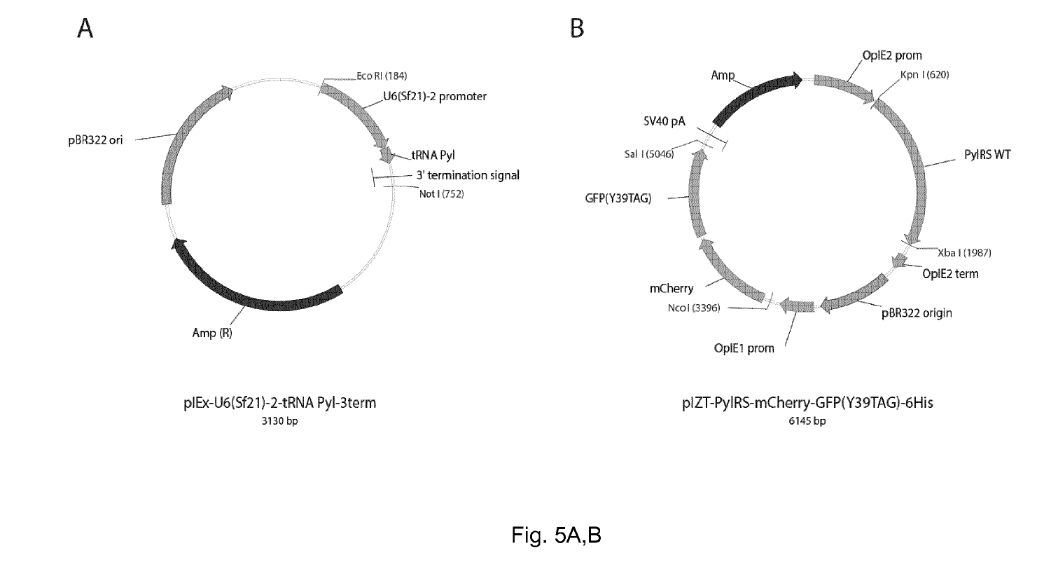

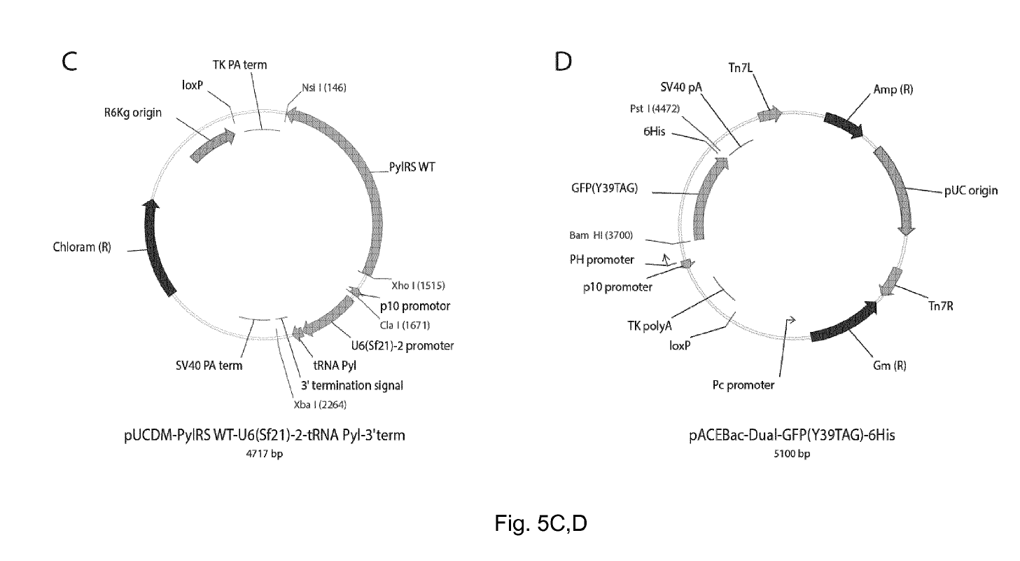

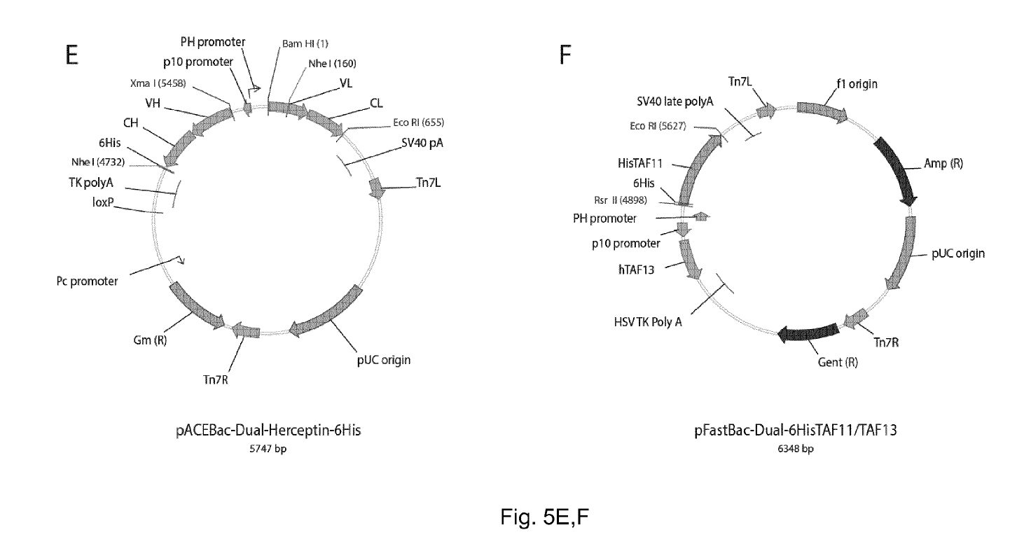

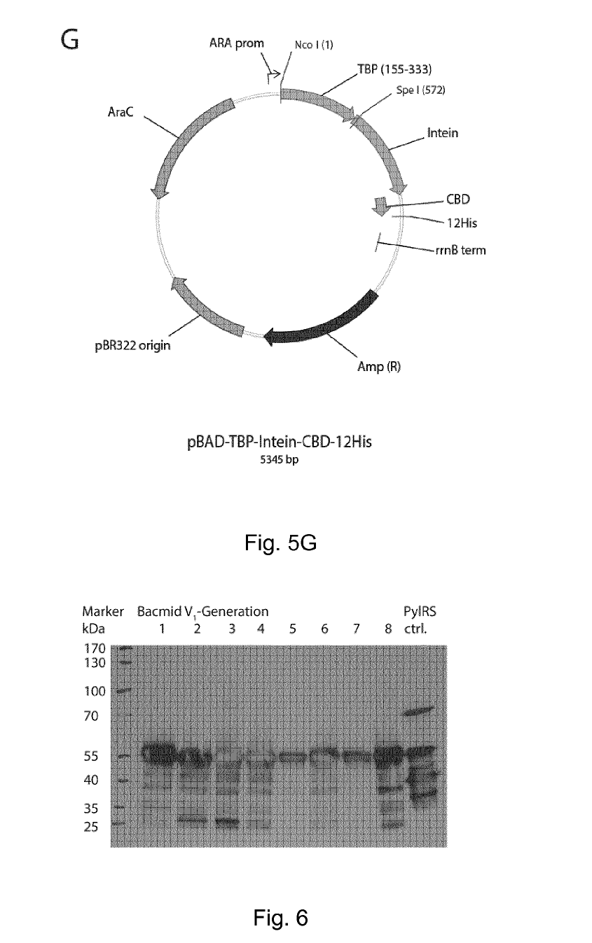

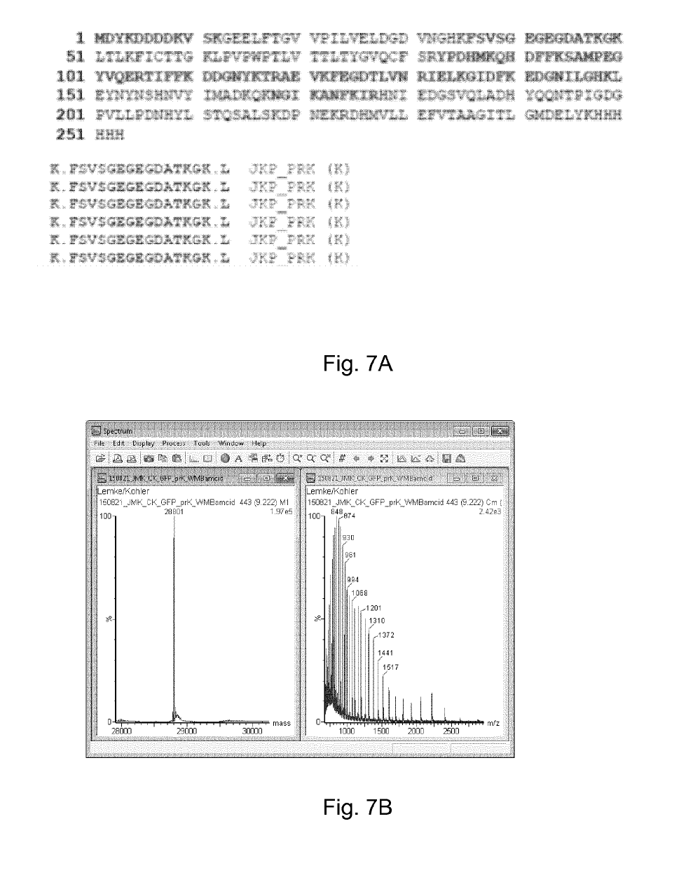

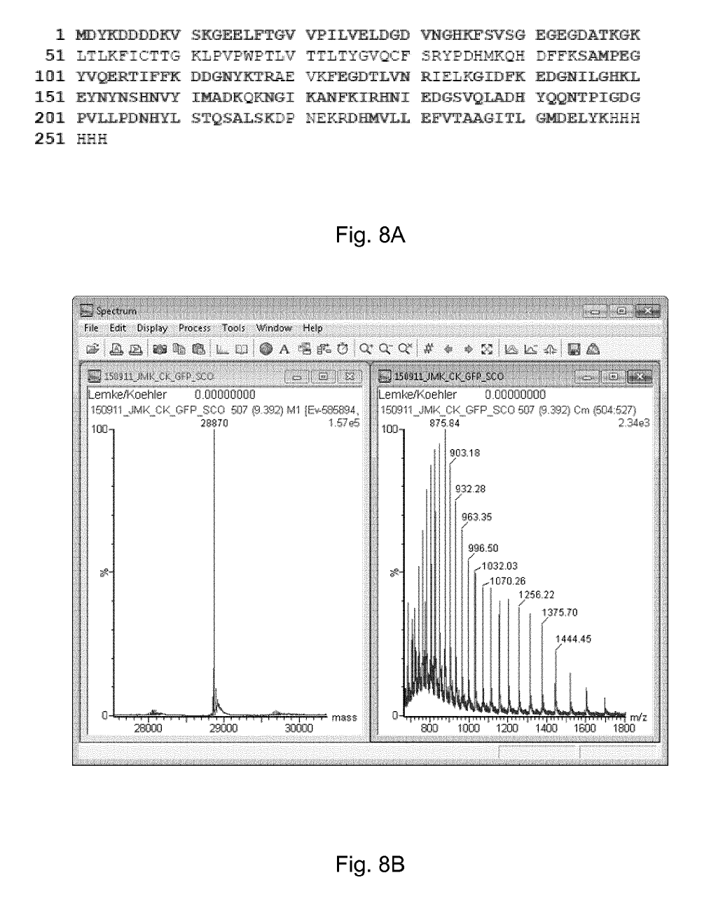

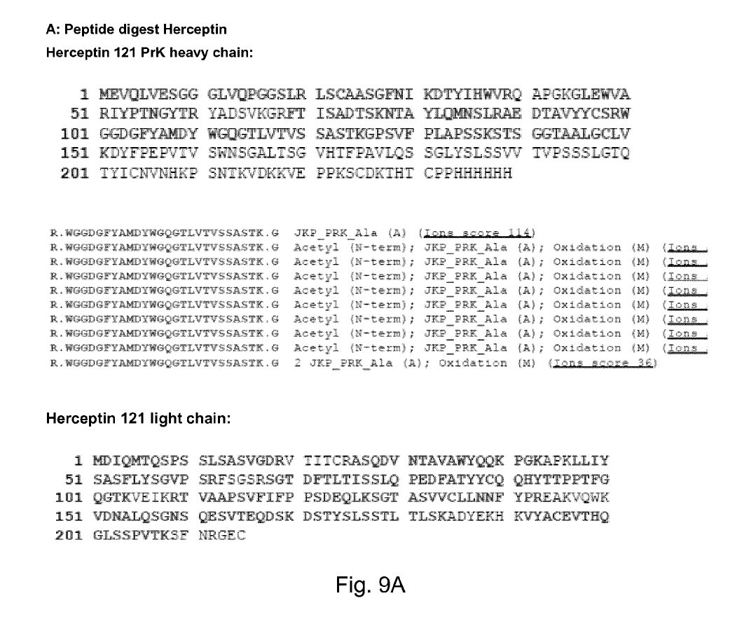

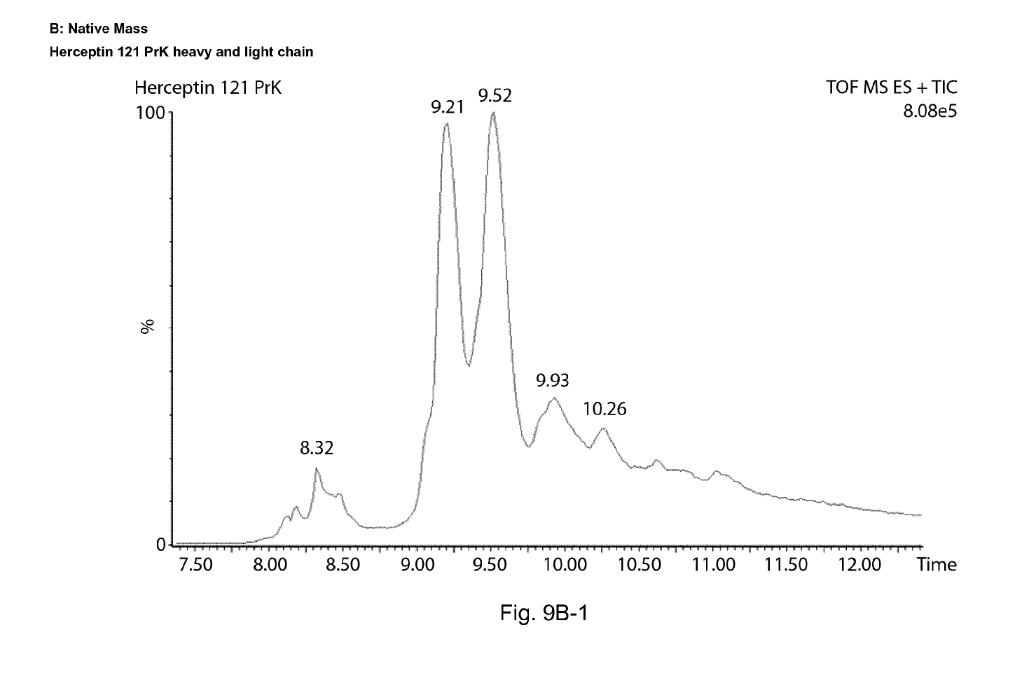

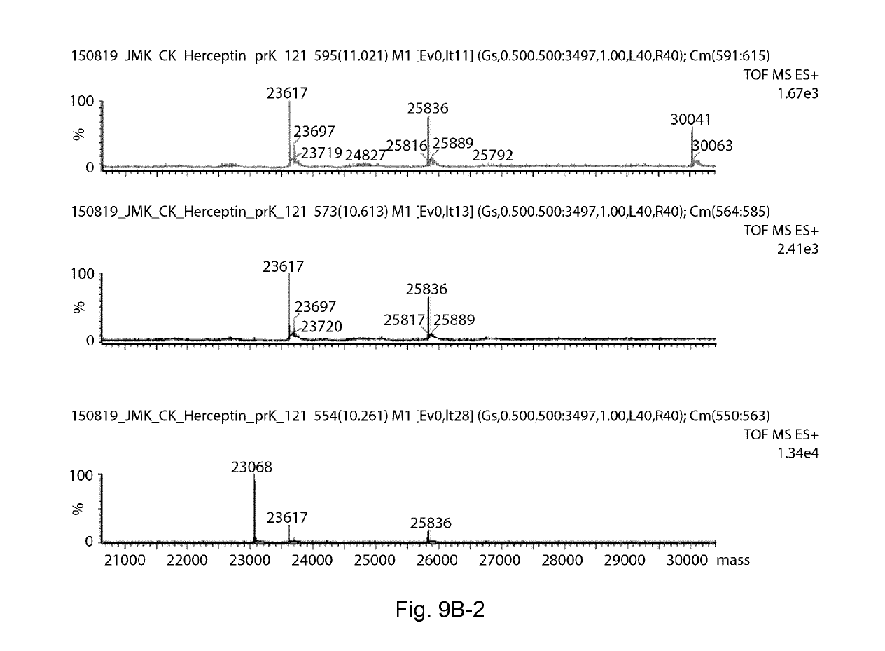

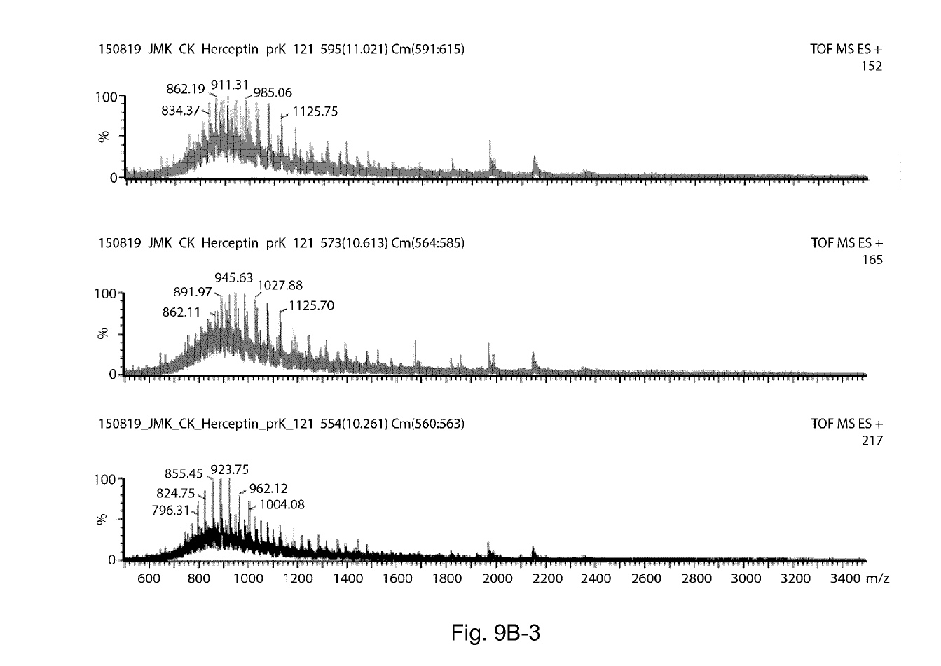

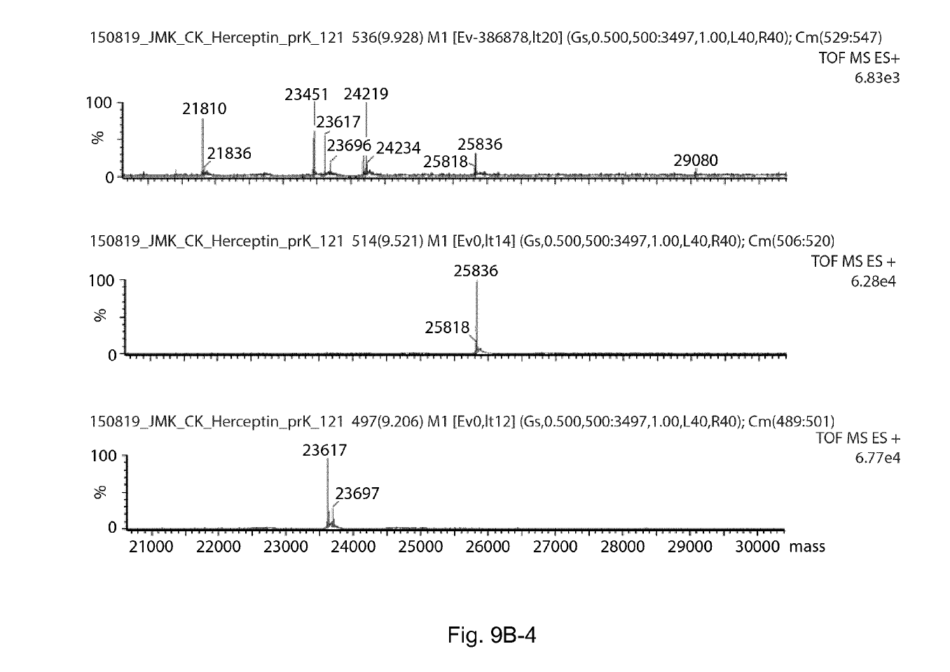

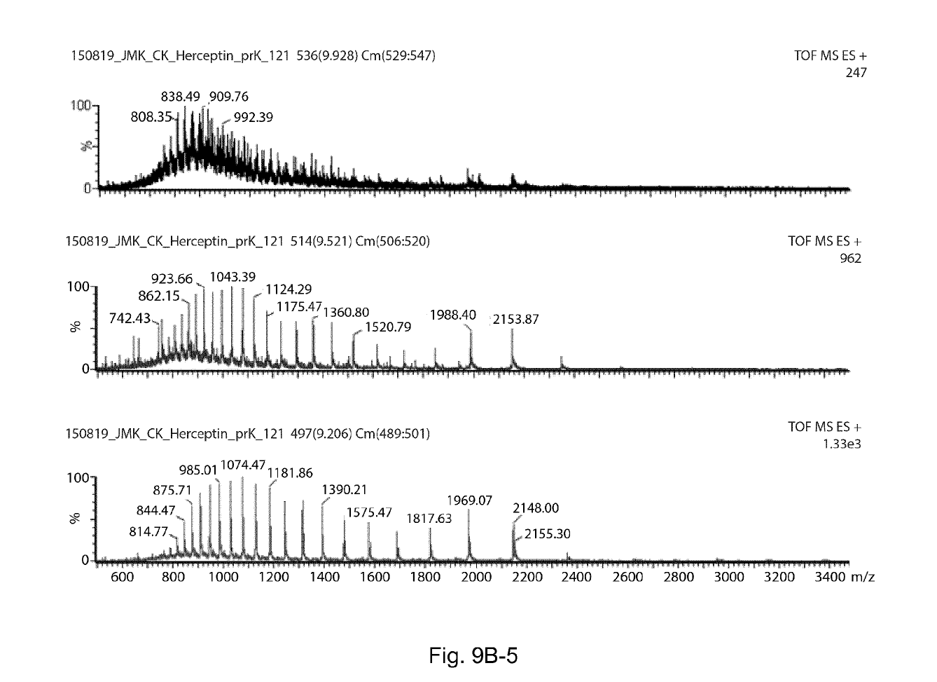

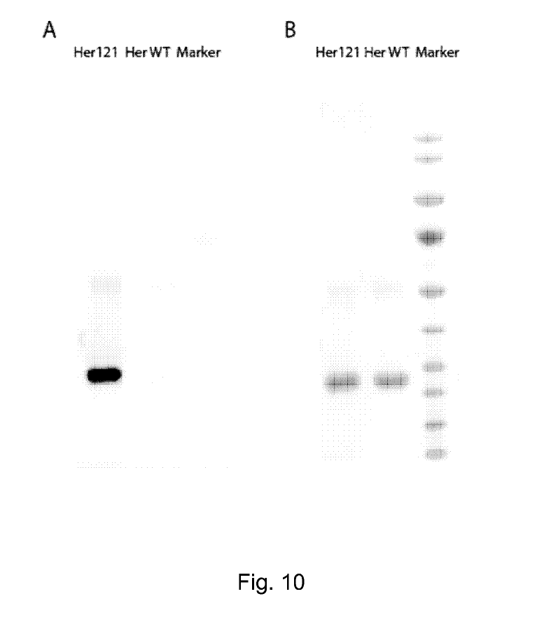

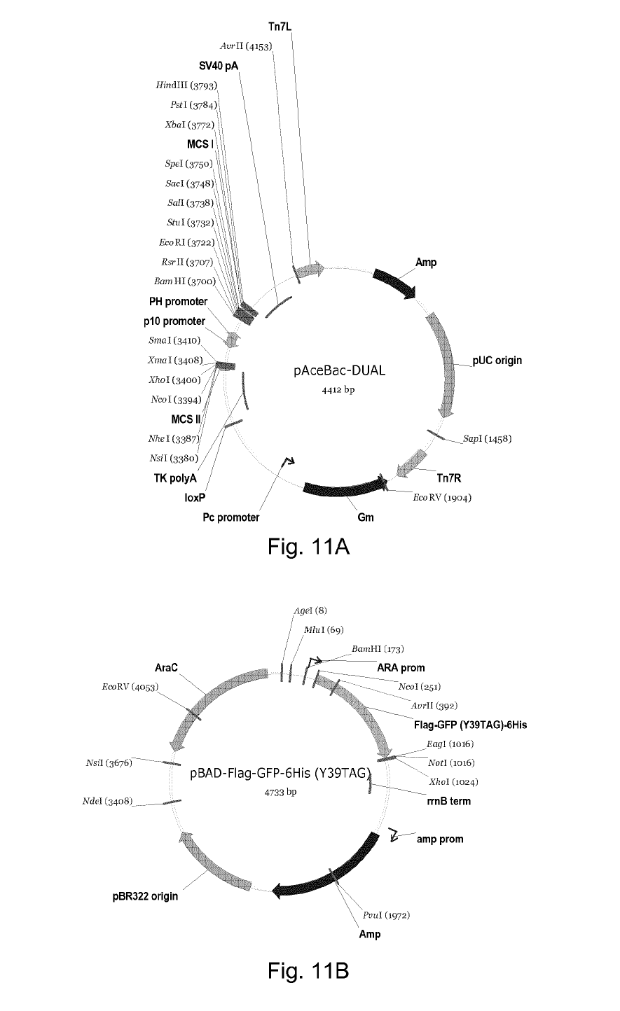

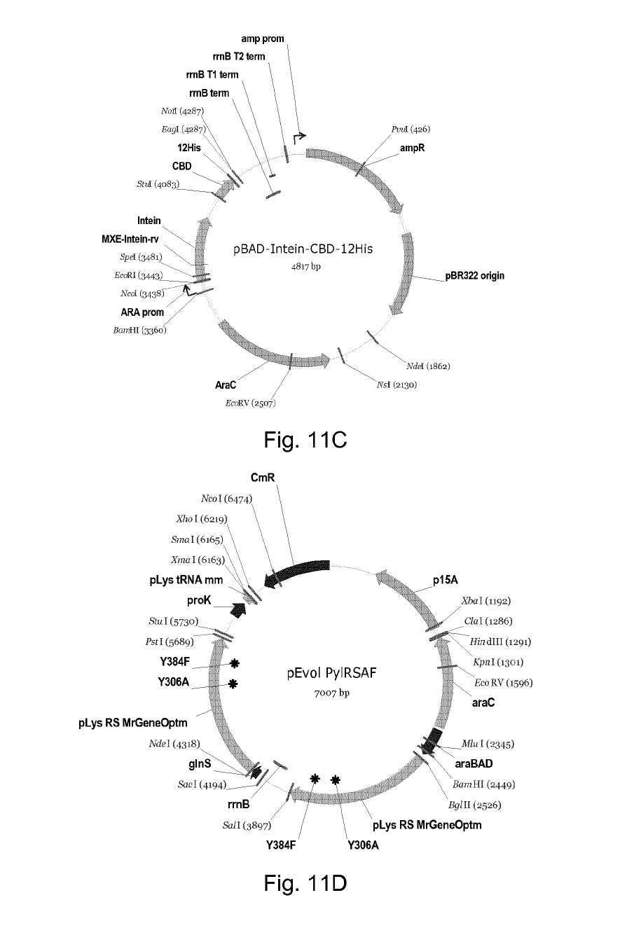

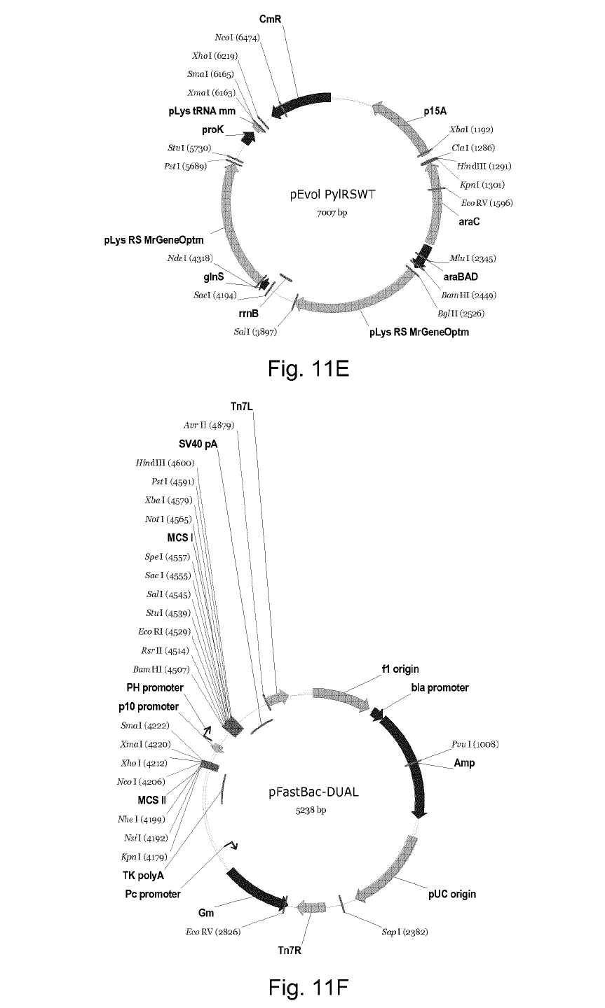

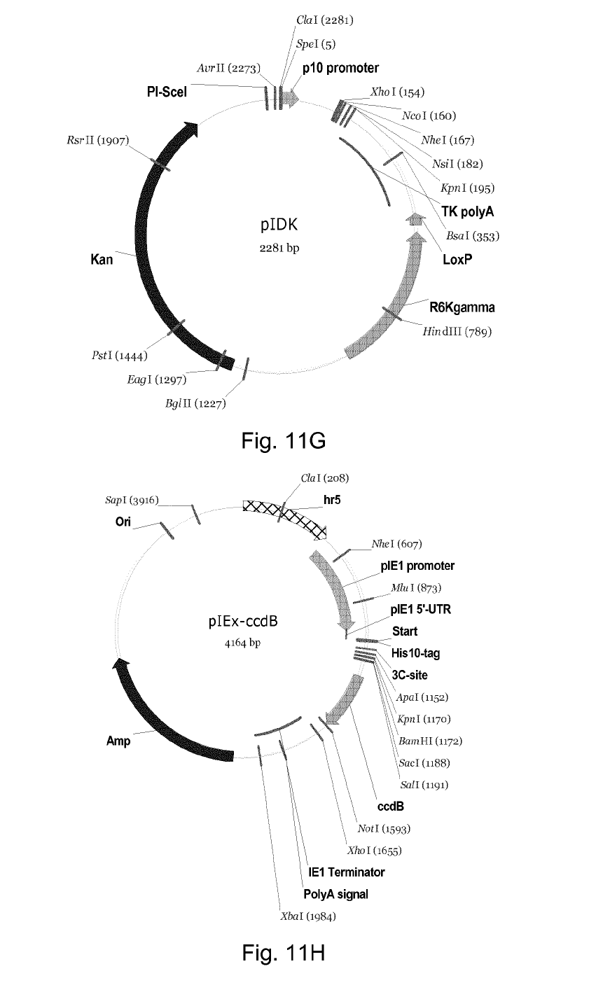

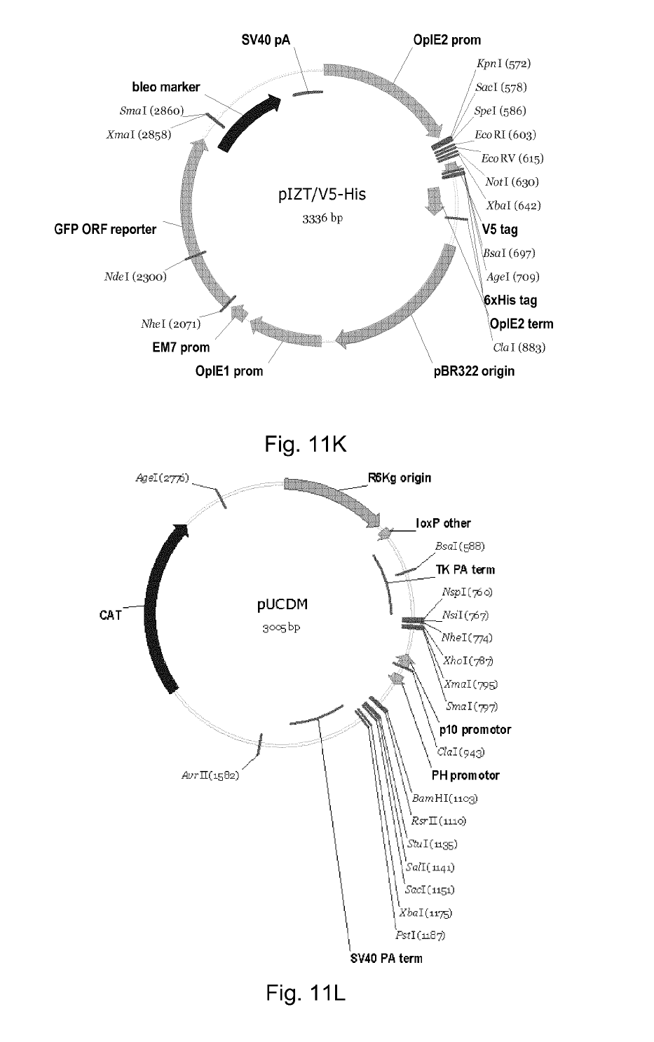

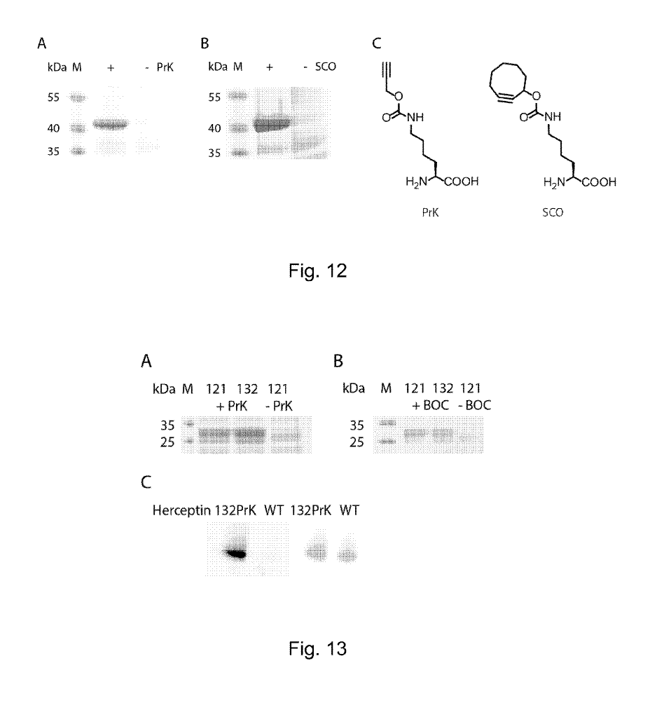

B: FACS results of Sf21 cells transfected with different U6-tRNA constructs and the reporter construct (pIZT-PylRSWT-mCherry-GFP(TAG) (FIGS. 3B-1 to 3B-4). Each analysis is represented by three plots. The upper right on shows scattering data and the selected gate, which contains live cells, as marked with a black eclipse. In the lower right plot singe cells are selected with a black eclipse. The final data is shown in the left diagram, which is divided into four gates. The upper left one shows the data point for cells, which only express mCherry (mCherry only), the lower left one shows cells, which are expressing neither mCherry nor GFP (double negatives). The lower right gate contains the cell counts for GFP expressing cells only (GFP only). In the upper right gate, cells are presented, which are expressing mCherry, as well as GFP, the so called double positives. The upper panel shows the expression results with PrK and the lower one without ncAA. FIG. 3B-1: Human U6-tRNAPyl-3term construct. FIG. 3B-2: U6 promoter from Drosophila melanogaster (Dm). FIG. 3B-3, U6 promoter from Bombyx mori (Bm)was used. FIG. 3B-4 shows the expression pattern using the U6 promoter from Spodoptera frugiperda (Sf21).

C: This figure shows a sequence-alignment-based comparison of different U6-tRNA^{Pyl} constructs (Promoter—tRNA^{Pyl} M. mazei—Termination signal), with promoter and termination signals of different origin (Human, D. melanogaster (Dm) and B. mori (Bm)); for S. frugiperda the U6-2 regulatory sequences are shown.

FIGS. 4-1 and 4-2 show the flow cytometry analysis of Schneider-2 cells transfected with pIZT-PylRS-mCherry-GFP(TAG) and pIEx-U6(Dm)-2-tRNA^{Pyl}-3term(1×) or pIEx-U6(Dm)-2-tRNA^{Pyl}-3term(4×) with and without ncAA are shown. Each analysis is represented by three plots. The upper right one shows scattering data and the selected gate, which contains live cells, as marked with a black eclipse. In the lower right plot singe cells are selected with a black eclipse. The final data is shown in the left diagram, which is divided into four gates. The upper left one shows the data point for cells, which only express mCherry (mCherry only), the lower left one shows cells, which are not expressing mCherry nor GFP (double negatives). The lower right gate contains the cell counts for GFP expressing cells only (GFP only). In the upper right gate, cells are presented, which are expressing mCherry, as well as GFP, the so called double positives. A (FIG. 4-1): Schneider-2 cells transfected with the 1× tRNA cassette with 1 mM PrK. B (FIG. 4-2): the 4× tRNA cassette was used for this transfection; C (FIG. 4-1): same as A, but without PrK; D (FIG. 4-2): same as B, but without PrK.

FIG. 5 shows in Panel A to F plasmid maps of plasmids used for transient transfections of Sf21 and S2R+ cells, as well as for the Baculovirus-system in Sf21 cells. All resistance gens, replication origins, other features, as well as protein or tRNA coding genes are highlighted. A: Plasmid map of pIEx-U6(Sf21)-2-tRNA^{Pyl}-3′term, containing under the U6(Sf21)-2 promoter the Mm tRNA^{Pyl} gen, ending with the U6-3′termination signal. B: This plasmid is built up of the Mm PylRS gen under the OpEI2 promoter, and under the OpEI1 promoter, the reporter construct mCherry-GFP(Y39TAG). C: The plasmid map of pUCDM-PylRS-U6(Sf21)-2-tRNA^{Pyl}-3′term shows the Mm PylRS gen under the p10 promoter and the tRNA^{Pyl} gen under the U6(Sf21)-2 promoter, followed by the 3′termination signal of U6(Sf21)-2. D: The gene for GFP(Y39TAG)-6His is cloned into the plasmid pACEBac-Dual under the PH promoter, further the plasmid contains two Tn7 sides, one Tn7L and one Tn7R, for the integration into the Bacmid. E: pACEBac-Dual-Herceptin-6His contains the F_{ab} fragment of Herceptin, the light chain (variable and constant region) under the PH promoter, the heavy chain (variable and constant region) under the p10 promoter with a C-terminal 6His tag. This plasmid also contains the Tn7 sides. F: The TAF complex, consisting of TAF11 and TAF13 is cloned into the pFastBac-Dual plasmid under the p10 and PH promoter, respectively. Tn7 sides are included in the map. G: Plasmid pBAD-TBP-Intein-CBD-12His includes the TBP gene for expression in E. coli, which has an Intein-CBD-12His tag, under an Ara promoter and the corresponding AraC gene.

FIG. 6 shows a western-blot of the expression of Mm PylRS in V,-stage using DH10BacTAG, using anti-PylRS as primary and anti-Rat-HRP conjugate as secondary antibody. The marker lane is drawn by hand after plotting and the molecular weights are indicated in kDa. Bacmid V₁-Generations 1-8 correspond to the eight different Bacmid-DNA preparations used. As a control PylRS-6His, expressed in E. coli and purified with standard Nickel purification protocol, was loaded (PylRS ctrl.).

FIG. 7 shows the mass spectrometry analysis of GFP(Y39PrK)

GFP(Y39PrK) was expressed in Sf21 cell, transfected with Bacmid-DNA prepared out of DH10Bac-TAG cells, harboring the MM PylRS WT and the tRNA^{Pyl} expression cassette. A: Result of the peptide digest using trypsin and the corresponding peptide sequence, showing the incorporation of PrK at position Y39 of GFP. B: Native mass result underlining the incorporation of PrK in the protein sequence.

FIG. 8 shows the mass spectrometry analysis of GFP(Y39SCO)

GFP(Y39SCO) was expressed in Sf21 cell, transfected with Bacmid-DNA prepared out of DH10Bac-TAG cells, harboring the MM PylRS AF and the tRNA^{Pyl} expression cassette. A: Result of the peptide digest using trypsin showing the coverage of the GFP sequence. B: Native mass result underlining the incorporation of SCO in the protein sequence.

FIG. 9 shows the mass spectrometry analysis of Herceptin 121 PrK

Herceptin (121 PrK) was expressed in Sf21 cell, transfected with Bacmid-DNA prepared out of DH10Bac-TAG cells, harboring the MM PylRS WT and the tRNA^{Pyl} expression cassette. A: Result of the peptide digest using trypsin showing the incorporation of PrK into the heavy chain of Herceptin at position 121 and the coverage of the light chain of Herceptin. B (FIGS. 9B-1 to 9B-5): Native mass result shows two main peaks (9.21 and 9.52) which correspond to the native mass of the light chain (23617) and the heavy chain (25836).

FIG. 10 shows the results of a SPACC click-reaction of Herceptin F_{ab} mutant 121SCO reacted with TAMRA-H-tetrazine and Herceptin F_{ab} wildtype. A: fluorescent scan; B: protein staining with Coomassie Blue. As can be seen Her121SCO is selectively marked.

FIGS. 11A to H, K and L shows the maps of different plasmids as applied in the context of the present invention.

FIG. 12 illustrates the expression of the amber mutant (Y39TAG) of GFP in the presence and absence of propargyl-lysine (PrK) and SCO-L-lysine; corresponding SDS-PAGE stained with Coomassie Blue are shown.

FIG. 13A: shows the SDS-PAGE of an Sf21 cell expression of Herceptin F_{ab} fragments carrying an amber stop codon at position 121 or 132 of the heavy chain with or without PrK; FIG. 13B: shows the expression of the same mutants with or without BOC-lysine; FIG. 13C illustrates via SDS-PAGE the selective alkyne-azide cycloaddition reaction for Herceptin F_{ab} fragment 132PrK mutant as compared to the wildtype (left panel; specific cycloaddition of mutant; right panel non-specific Coomassie Blue staining).

### DETAILED DESCRIPTION OF THE INVENTION

### A. General Definitions and Abbreviations

1. Definitions

If not otherwise stated nucleotide sequences (NS) are depicted herein in the 5′→3′ direction. If not otherwise stated amino acid sequences (AS) are depicted herein in the N-terminal 4 to C-terminal direction.

The term “ncAA” refers generally to any non-canonical or non-natural amino acid or amino acid residue which is not among the 22 naturally occurring proteinogenic amino acids. Numerous ncAAs are well known in the art (for reviews see: Liu, C. C., and Schultz, P. G. (2010). Annual review of biochemistry 79, 413-444; Lemke, E. A. (2014). Chembiochem: a European journal of chemical biology 15, 1691-1694). Particular preferred ncAAs are those which may be post-transitionally further modified.

The term “translation system” generally refers to a set of components necessary to incorporate a naturally occurring amino acid in a growing polypeptide chain (protein). Components of a translation system can include, e.g., ribosomes, tRNAs, aminoacyl tRNA synthetases (RS), mRNA and the like.

An aminoacyl tRNA synthetase (RS) is an enzyme capable of acylating a tRNA with an amino acid or amino acid analog. An RS used in processes of the invention is capable of acylating a tRNA with the corresponding ncAA, i.e. acylating a tRNA^{ncAA}.

The term “orthogonal” as used herein refers to a molecule (e.g., an orthogonal tRNA (O-tRNA) and/or an orthogonal aminoacyl tRNA synthetase (O-RS)) that is used with reduced efficiency by a translation system of interest (e.g., a cell). “Orthogonal” refers to the inability or reduced efficiency, e.g., less than 20% efficient, less than 10% efficient, less than 5% efficient, or e.g., less than 1% efficient, of an orthogonal tRNA or an orthogonal aminoacyl tRNA synthetase to function with the endogenous aminoacyl tRNA synthetases or endogenous tRNAs, respectively, of a translation system of interest.

For example, an orthogonal tRNA (O-tRNA) in a translation system of interest is acylated by any endogenous aminoacyl tRNA synthetase of a translation system of interest with reduced or even zero efficiency, when compared to acylation of an endogenous tRNA by the endogenous aminoacyl tRNA synthetase. In another example, an orthogonal aminoacyl tRNA synthetase (O-RS) acylates any endogenous tRNA in the translation system of interest with reduced or even zero efficiency, as compared to acylation of the endogenous tRNA by an endogenous aminoacyl tRNA synthetase.

“Orthogonal RS/tRNA pairs” or “O-tRNA/O-RS pairs” used in processes of the invention preferably have following properties: the O-tRNA is “preferentially acylated” with the unnatural amino acid of the invention by the O-RS. In addition, the orthogonal pair functions in the translation system of interest, e.g., the translation system uses the unnatural amino acid (ncAA) acylated O-tRNA to incorporate the unnatural amino acid (ncAA) in a polypeptide chain.

Incorporation occurs in a site-specific manner, e.g., the O-tRNA recognizes a “selector codon”, e.g., an amber stop codon, in the mRNA coding for the polypeptide. As non-limiting examples there may be mentioned TyrRS/tRNA^{Tyr} from B. stearothermophilus; TrpRS/tRNA^{Trp} from B. subtilis or PylRS/tRNA^{Pyl} from M. mazei.

The term “preferentially acylated” refers to an efficiency of, e.g., about 50% efficient, about 70% efficient, about 75% efficient, about 85% efficient, about 90% efficient, about 95% efficient, or about 99% or more efficient, at which an O-RS acylates an O-tRNA with an unnatural amino acid (ncAA) compared to an endogenous tRNA or amino acid of a translation system of interest. The ncAA is then incorporated in a growing polypeptide chain with high fidelity, e.g., at greater than about 75% efficiency for a given selector codon, at greater than about 80% efficiency for a given selector codon, at greater than about 90% efficiency for a given selector codon, at greater than about 95% efficiency for a given selector codon, or at greater than about 99% or more efficiency for a given selector codon.

The term “selector codon” refers to any codon (any stop codon, any coding codon, or quadruplet codon) recognized by the O-tRNA in the translation process and not recognized by an endogenous tRNA. The O-tRNA anticodon loop recognizes the selector codon on the mRNA and incorporates its amino acid, e.g., an ncAA, at this site in the polypeptide. Selector codons can include, e.g., nonsense codons, such as stop codons, e.g., amber, ochre, and opal codons; four or more base codons; codons derived from natural or unnatural base pairs and the like. For a given system, a selector codon can also include one of the natural three base codons (i.e. natural triplets), wherein the endogenous system does not use said natural triplet, e.g., a system that is lacking a tRNA that recognizes the natural triplet or a system wherein the natural triplet is a rare codon.

An “anticodon” has the reverse complement sequence of the corresponding codon.

An “O-RS/O-tRNA” pair is composed of an O-tRNA, e.g., a suppressor tRNA, or the like, and an O-RS.

A “suppressor tRNA” is a tRNA that alters the reading of a messenger RNA (mRNA) in a given translation system. A suppressor tRNA can read through, e.g., a stop codon, a four base codon, or a rare codon.

The O-tRNA is not acylated by endogenous synthetases and is capable of decoding a selector codon, as described herein. The O-RS recognizes the O-tRNA, e.g., with an extended anticodon loop, and preferentially acylates the O-tRNA with an unnatural amino acid (ncAA).

“Bacterial” in the context of the present invention has to be understood broadly and encompasses bacteria of the class Bacteria, and for example of the families Coccaceae, Bacteriaceae, Bacillaceae, Spirillaceae or of the class Cyanophycea; as well as Archaebacteria (Archaea) as for example of the families Caldisphaeraceae, Cenarchaeaceae, Desulfurococcaceae, Pyrodictiaceae, Sulfolobaceae, Thermoproteaceae, Thermofilaceae, Nitrososphaeraceae, Archaeoglobaceae, Halobacteriaceae, Methanobacteriaceae, Methanothermaceae, Methanocaldococcaceae, Methanococcaceae, Methanocellaceae Methanocorpusculaceae, Methanomicrobiaceae, Methanospirillaceae, Methanosaetaceae, Methanosarcinaceae, Methermicoccaceae, Methanopyraceae, Thermococcaceae, Ferroplasmataceae, Picrophilaceae or Thermoplasmataceae.

“Amber suppression” in the context of the invention has to be understood broadly, if not otherwise stated, and is understood as any type reprogramming any codon (such as natural codons, quadruplet codons) aiming at the introduction of ncAAs into a polypeptide or protein. More narrowly, said term is synonymously used with the term stop codon suppression.

“Genetic code expansion” refers to reprogramming any codon aiming at the introduction of ncAAs into a polypeptide or protein.

An “amber-suppressor” refers to a gene whose gene product suppresses in a cell the action of an amber mutation, in particular a mutation resulting in the generation of a preterm stop codon. In presence of an amber suppressor cells will again synthesize complete functional, biologically active polypeptides. Amber suppressors in the context of the invention are tRNA whose anticodon recognizes the amber (stop) codon as amino acid codon, here the codon of a non-naturally occurring amino acid (ncAA). Thus, the amber codon does no longer result in preterm chain termination during biosynthesis of the gene product.

“snRNA” refers to small nuclear ribonucleic acids with a sequence length of 100 to 300 residues; they are localized in the cell nucleus, are produced by RNA Polymerase II and III, There are several different types of snRNAs, and the snRNAs U1, U2, U4, U6 and U5 are involved in mRNA splicing processes. snRNAs are catalytically active and responsible for the spicing of introns of pre-mRNA in the cell nucleus.

“Baculovirus” (“bacmid”) refers to an expression vector system which shows several advantageous features: High levels of heterologous gene expression are often achieved compared to other eukaryotic expression systems, particularly for intracellular proteins. In many cases, the recombinant proteins are soluble, post-translationally modified and easily recovered from infected cells late in infection when host protein synthesis is diminished. The cell lines used for propagation grow well in suspension cultures, permitting the production of recombinant proteins in large-scale bioreactors. Expression of hetero-oligomeric protein complexes can be achieved by simultaneously infecting cells with two or more viruses or by infecting cells with recombinant viruses containing two or more expression cassettes. Baculoviruses have a restricted host range, limited to specific invertebrate species. They are safer to work with than most mammalian viruses since they are noninfectious to vertebrates. Particular baculovirus vectors are those shuttle vectors that can be propagated in both E. coli and insect cells. Baculovirus vector systems are generally known and commercially available. Reference is for example made to the so-called multibac® expression system specifically suited for multigene applications, which comprises a modified Baculovirus recipient DNA and a set of baculovirus transfer vectors which allow a simple and rapid transfer of multiple coding sequences into specific sites of the recipient DNA in E. coli. Said thus modified baculoviral recipient DNA may the be propagated and isolated and applied as vector for the transfection of suitable hosts wherein protein expression is then performed. “Baculovirus” (“bacmid”) vectors encompass isolated baculoviral DNA as well as viral vectors carrying the same.

“Insect-cell derived” refers to genes or gene products which naturally contained in or produced by an insect cell or insect cell line.

“Transfection” refers to the direct gene transfer (for example of viral DNA) into eukaryotic cells, like for example insect cells. Said term has to be understood broadly and also encompasses the gene transfer) into eukaryotic cells, like for example insect cells, by the process of “transduction” i.e. by viral infection.

2. Abbreviations
TP target polypeptide
ncAA, non-canonical amino acid residues
ICL insect cell line
CSTP TP encoding nucleotide sequence
O-RS orthogonal bacterial aminoacyl tRNA synthetase
O-tRNA^{ncAA} orthogonal bacterial tRNA^{ncAA}
CS tRNA^{ncAA} coding sequences of said one or more bacterial tRNA^{ncAA}
RS^{IC} insect-cell derived regulatory sequence and
snRNA small nuclear ribonucleic acid
tRNA transfer ribonucleic acid
tRNA^{Pyl} Pyrrolysyl tRNA
PylRS Pyrrolysyl tRNA Synthetase
PylRS WT Pyrrolysyl tRNA Synthetase wild-type
PylRS AF Pyrrolysyl tRNA Synthetase mutant
Pyl Pyrrolysin
SCO particular cyclooctynyl derivative of Lysine
TCO particular cyclooctynyl derivative of Lysine
TOC* particular cyclooctynyl derivative of Lysine
BOC Butoxycarbonyl lysine
PrK particular propynyl derivative of lysine
GFP green fluorophore (Green fluorescent protein from Aequoria victoria)
mCherry red fluorophore (from Drosophila, monomeric form)

### B. Particular Embodiments

The present invention relates to the following particular embodiments.
1. A method of preparing a, in particular engineered, target polypeptide (TP) comprising in its amino acid sequence one or more, identical or different, preferably 1 to 5, most preferably 1, 2 or 3, non-canonical amino acid (ncAA) residues, which method comprises the steps of
   a) expressing said TP in an insect cell line (ICL), in the presence of said one or more ncAAs, wherein the TP encoding nucleotide sequence (CS^{TP}) comprises one or more, preferably 1 to 5, most preferably 1, 2 or 3, selector codons encoding said one or more ncAA residues; and concomitantly or sequentially in any order
   b) expressing in said ICL one or more, preferably 1 to 5, most preferably 1, 2 or 3, orthogonal bacterial, in particular archaebacterial, aminoacyl tRNA synthetase/tRNA^{ncAA} (O-RS/O-tRNA^{ncAA}) pairs required for introducing said one or more ncAA residues into the amino acid sequence of said TP, wherein the coding sequences for said one or more bacterial, in particular archaebacterial, tRNA^{ncAA} (CS^{tRNAncAA}) are under the control of an insect-cell derived regulatory sequence (RS^{IC}); and
   c) optionally recovering the expressed TP.

Said TP corresponds to a native or parent eukaryotic or a prokaryotic polypeptide which native or parent polypeptide distinguishes from TP in that it does not contain an ncAA. A TP may be composed of one single or more, identical or different polypeptide chains. The expressed TP may be in the form of homo- or heterooligomeric protein complexes (aggregates) the polypeptide chains of which adhere together by non-covalent (for example ionic and/or hydrophobic interactions) or are covalently linked, for example via disulphide bridges.

Preferred archaebacterial tRNA^{ncAA} are in particular so-called polyspecific tRNA^{ncAA} which may be aminoacylated with different ncAAs, so that in a particular selector codon, like an amber stop codon, a ncAA selected from a set of different ncAAs may be inserted in the corresponding sequence position of the TP to be expressed. Non limiting examples are tRNA^{Pyl} from M. mazei or tRNA^{Tyr} from B. stearothermophilus.
2. The method of embodiment 1, wherein said ICL is transiently or stably, preferably stably, transfected with one or more, like 1 to 5, preferably 1, 2 or 3, most preferably 1 or 2, vectors carrying the genetic information (coding sequences and regulatory sequences) required for expressing said TP and said one or more O-RS/O-tRNA^{ncAA} pairs.
3. The method of one of the preceding embodiments, wherein said ICL is transfected with one or more, like 1 to 5, preferably 1, 2 or 3, most preferably 1 or 2, baculoviral vectors carrying the genetic information (coding sequences and regulatory sequences) required for expressing said TP and said one or more O-RS/O-tRNA^{ncAA} pairs
4. The method one of the preceding embodiments, wherein said RS^{IC} and said ICL are derived from identical insect species, insect of the genus Spodoptera, preferably Spodoptera frugiperda, like in particular Spodoptera frugiperda cell line Sf21 (DSMZ Nr. ACC119).
5. The method of one of the preceding embodiments, wherein said RS^{IC} is derived from an insect of the genus Spodoptera, preferably Spodoptera frugiperda, like in particular Spodoptera frugiperda cell line Sf21 (DSMZ Nr. ACC119).
6. The method of one of the preceding embodiments, wherein said RS^{IC} is selected from regulatory sequences recognized by (is functional for) RNA-polymerase III, in particular insect RNA-polymerase III.
7. The method of embodiment 4, wherein said RS^{IC} is selected from
   a) regulatory sequences of insect snRNA U6 genes, and
   b) regulatory sequences of insect tRNA genes, in particular H1 regulatory sequences; wherein embodiment a) is preferred.
8. The method of one of the preceding embodiments, wherein said RS^{IC} comprises at least one, preferably one U6 promoter sequence 5′-downstream of the insect snRNA U6 coding sequence, in particular 5′-downstream of the insect snRNA U6 coding sequence as depicted in FIG. 1, preferably 5′-downstream of the insect snRNA U6 coding sequence of SEQ ID NO:12.
9. The method of embodiment 8, wherein said U6 promoter is selected from nucleotide sequences corresponding to consecutive nucleotide residues
   a) 1 to 400 of SEQ ID NO. 1
   b) 1 to 392 of SEQ ID NO: 2
   c) 1 to 385 of SEQ ID NO: 3 to 8
   d) functional fragments thereof, which retain the intended promoter activity, and in particular regulate the expression of tRNA^{ncAA} being under the control for said functional fragment, comprising a partial sequence of anyone of the nucleotide sequences as defined in a) to c)
   Preferably said U6 promotor may be selected from sequences of groups b) or c); more preferably from b) or c), SEQ ID NO: 4, 5, 6, 7 or 8.
   By routine experimentation, optionally based on further suitable sequence information, like suitable sequence alignments of related sequences, as for example like those depicted in FIG. 3 C, a skilled reader may provide, without undue experimentation, further suitable promoter sequences, which differ in sequence from those mentioned under items a), b) and c) of embodiment 9, while retaining the intendent promoter activity of regulation the intended expression of tRNA^{ncAA} being under the control for said functional fragment.

For example suitable functional fragments of promoters may comprise less that 400 consecutive nucleotide residues, less than 300, less than 250, less than 200, less than 150, or less than 100 and at least 20, at least 30, at least 40, at least 50, at least 60, at least 70 or at least 80 or at least 90 nucleotide residues preferably 5′-downstream of the insect snRNA U6 coding sequence of SEQ ID NO:12, which may be a single fragment of consecutive 5′-downstream residues or may encompass more that one partial sequence comprising one or more functional partial elements, required for the intended promoter activity. As non-limiting examples of such functional partial elements there may be mentioned TATA boxes or PSEA elements as described in the art.
For example suitable functional fragments of promoters may have a sequence length of 20, 30, 40 or 50 to 399, like 20 to 350, 20 to 300, 20 to 250, 20 to 200, 20 to 150, 20 to 100, or like 30 to 350, 30 to 300, 30 to 250, 30 to 200, 30 to 150, 30 to 100, or like 40 to 350, 40 to 300, 40 to 250, 40 to 200, 40 to 150, 40 to 100; or like 50 to 350, 50 to 300, 50 to 250, 50 to 200, 50 to 150, 50 to 100 nucleotide residues; or like 100 to 350, 100 to 300, 100 to 250, 100 to 200, 100 to 150, nucleotide residues; or like 80 to 350, 80 to 300, 80 to 250, 80 to 200, 80 to 150, 80 to 100 nucleotide residues.
10. The method of one of the preceding embodiments, wherein said RS^{IC} comprises a U6 termination sequence 3′-upstream of the of insect snRNA U6 coding sequence, in particular 3′-upstream of the insect snRNA U6 coding sequence as depicted in FIG. 1, preferably 3′-upstream of the insect snRNA U6 coding sequence of SEQ ID NO:12.
11. The method of embodiment 10, wherein said U6 terminator is selected from nucleotide sequences corresponding to consecutive nucleotide residues
   a) 470 to 569 of SEQ ID NO:1
   b) 462 to 561 of SEQ ID NO:2
   c) 455 to 554 of SEQ ID NO:3 to 8
   d) functional fragments thereof, preferably which retain the intended terminator activity, and in particular regulate the expression of tRNA^{ncAA} being under the control for said functional fragment, comprising a partial sequence of anyone of the nucleotide sequences as defined in a) to c).
   Preferably said U6 promotor may be selected from sequences of groups b) or c); more preferably from b) or c), SEQ ID NO: 4, 5, 6, 7 or 8
   By routine experimentation, optionally based on further suitable sequence information, like suitable sequence alignments of related sequences, as for example like those depicted in FIG. 3C, a skilled reader may provide, without undue experimentation, further suitable terminator sequences, which differ in sequence from those mentioned under items a), b) and c) of embodiment 11, while retaining the intendent terminator activity of regulation the intended expression of tRNA^{ncAA} being under the control for said functional fragment.
   For example suitable functional fragments of terminators may comprise less than 100 consecutive nucleotide residues, less than 75, lest than 50, less than 40, less than 30, less than 30 and at least 10, at least 15, at least 20, at least 30 nucleotide residues preferably 3′-downstream of the insect snRNA U6 coding sequence of SEQ ID NO:12, which may be a single fragment of consecutive 3′-upstream residues or may encompass more than one partial sequence comprising one or more functional partial elements, required for the intended terminator activity.
   For example suitable functional fragments of promoters may have a sequence length of 10, 20, 30, 40 or 50 to 99, like 10 to 90, 10 to 80, 10 to 70, 10 to 60, 10 to 50, 10 to 40, or like 20 to 90, 20 to 80, 20 to 70, 20 to 60, 20 to 50, 20 to 40, or like 30 to 90, 30 to 80, 30 to 70, 30 to 60, 30 to 50, 30 to 40, nucleotide residues.
12. The method of embodiment 1, wherein an expression cassette for bacterial, in particular archaebacterial tRNA^{ncAA} is applied which is construed according to the following scheme:
   5′-downstream U6 promoter/tRNA^{ncAA} coding sequence/3′-upstream U6 terminator
13. The method of one of the preceding embodiments, wherein the bacterial tRNA^{ncAA} is of archaebacterial origin, in particular of a bacterium of the genus Methanosarcina, like M. M. hafniense, M. bakeri and M. mazei, preferably M. mazei.
14. The method of one of the preceding embodiments, wherein said bacterial tRNA^{ncAA} is Pyrrolysyl tRNA (tRNA^{Pyl}) from Methanosarcina mazei.
15. The method of embodiment 14, wherein said bacterial tRNA^{Pyl} is encoded by an expression cassette comprising a nucleotide sequences selected from U6-1 to U6-8 according to SEQ ID NOs: 1 to 8 or sequences having a degree of sequence identity of at least 40%, as for example at least 50, 60, 70, 75, 80, 85, 90, 91, 92, 93, 94, 95, 96, 97, 98, or 99%, retaining their ability to functionally express bacterial, in particular archaebacterial, tRNA^{Pyl} in insect cells, in particular Spodoptera cell lines, preferably Spodoptera frugiperda cell lines.
16. The method of embodiment 14 or 15 wherein said tRNA^{Pyl} comprises a nucleotide sequence according to SEQ ID NO: 9 or a sequence having a degree of sequence identity of at least 70%, as for example at least 75, 80, 85, 90, 91, 92, 93, 94, 95, 96, 97, 98, or 99%, while retaining the tRNA^{Pyl} function.
17. The method of one of the preceding embodiments, wherein said bacterial O-RS is of archaebacterial origin, and preferably is PylRS comprising an amino acid sequence of SEQ ID NO: 27 or a sequence having a degree of sequence identity of at least 70%, as for example at least 75, 80, 85, 90, 91, 92, 93, 94, 95, 96, 97, 98, or 99%, while retaining the PylRS function, in particular PylRS WT SEQ ID NO: 27 or PylRS AF SEQ ID NO: 30.
18. The method of anyone of the preceding embodiments, wherein said ICL is selected from Spodoptera cell lines, in particular Spodoptera frugiperda cell lines, preferably Sf21 (DSMZ Nr. ACC119); or Drosophila cell lines, in particular Drosophila melanogaster cell lines preferably Schneider-2 R+ (Drosophila Genomics Research Center (DGRC) stock number 150) or Schneider 2 (ATCC CRL-1963). Preferably, said ICL is selected from Spodoptera cell lines, more preferably Spodoptera frugiperda cell lines, most preferably Sf21 (DSMZ Nr. ACC119). Further said ILC may be selected from Trichoplusia cell lines, preferably Trichoplusia ni BTI-Tn-5B1-4 (High Five, Invitrogen).
19. A baculoviral shuttle vector (bacmid) (encompassing isolated baculoviral DNA or the corresponding viral vector carrying said DNA) comprising the coding sequences of one or more orthogonal bacterial, in particular archaebacterial, aminoacyl tRNA synthetase/tRNA^{ncAA} (O-RS/O-tRNA^{ncAA}) pairs, wherein said bacterial, in particular archaebacterial, tRNA^{ncAA} coding sequence (CS^{tRNAncAA}) is placed under the control of an insect-cell derived regulatory sequence (RS^{IC}).
20. The vector of embodiment 19, wherein said RS^{IC} is selected from regulatory sequences recognized by (is functional for) RNA-polymerase III, in particular insect RNA-polymerase III.
21. The vector one of the embodiments 19 or 20, wherein said RS^{IC} is derived from an insect of the genus Spodoptera, preferably Spodoptera frugiperda, like in particular Spodoptera frugiperda cell line Sf21 (DSMZ Nr. ACC119).
22. The vector of one of the embodiments 19 to 21, wherein said RS^{IC} is selected from
   a) regulatory sequences of insect snRNA U6 genes, and
   b) regulatory sequences of insect tRNA genes, in particular H1 regulatory sequences; wherein embodiment a) is preferred
23. The vector of one of the embodiments 19 to 22, wherein said RS^{IC} comprises a U6 promoter sequence 5′-downstream of the insect snRNA U6 coding sequence, in particular 5′-downstream of the insect snRNA U6 coding sequence as depicted in FIG. 1, preferably 5′-downstream of the insect snRNA U6 coding sequence of SEQ ID NO:12.
24. The vector of embodiment 23, wherein said U6 promoter is selected from nucleotide sequences corresponding to nucleotide residues
   a) 1 to 400 of SEQ ID NO. 1
   b) 1 to 392 of SEQ ID NO: 2
   c) 1 to 385 of SEQ ID NO: 3 to 8
   d) functional fragments thereof which retain the intended promoter activity, and in particular regulate the expression of tRNA^{ncAA} being under the control for said functional fragment, comprising a partial sequence of anyone of the nucleotide sequences as defined in a) to c)

As regards the provision of further functional fragments reference is made to embodiment 9, above.
25. The vector of one of the embodiments 19 to 24, wherein said RS^{IC} comprises a U6 termination sequence 3′-upstream of the of insect snRNA U6 coding sequence, in particular 3′-upstream of the insect snRNA U6 coding sequence as depicted in FIG. 1, preferably 3′-upstream of the insect snRNA U6 coding sequence of SEQ ID NO:12.
26. The vector of embodiment 25, wherein said U6 terminator is selected from nucleotide sequences corresponding to nucleotide residues
   a) 470 to 569 of SEQ ID NO:1
   b) 462 to 561 of SEQ ID NO:2
   c) 455 to 554 of SEQ ID NO:3 to 8
   d) functional fragments thereof, preferably which retain the intended terminator activity, and in particular regulate the expression of tRNA^{ncAA} being under the control for said functional fragment, comprising a partial sequence of anyone of the nucleotide sequences as defined in a) to c).

As regards the provision of further functional fragments reference is made to embodiment 11, above.
27. The vector of anyone of the embodiments 19 to 26, comprising an expression cassette for bacterial, in particular archaebacterial, tRNA^{ncAA}, which is construed according to the following scheme:
   5′-downstream U6 promoter/tRNA^{ncAA} coding sequence/3′-upstream U6 terminator
28. The vector of anyone of the embodiments 19 to 27, wherein the bacterial tRNA^{ncAA} is of archaebacterial origin, in particular of a bacterium of the genus Methanosarcina, like M. M. hafniense, M. bakeri and M. mazei, preferably Methanosarcina mazei.
29. The vector of anyone of the embodiments 19 to 28, wherein said bacterial tRNA^{ncAA} is Pyrrolysyl tRNA (tRNA^{Pyl}) from Methanosarcina mazei.
30. The vector of anyone of the embodiments 19 to 29, wherein said bacterial tRNA^{Pyl} is encoded by an expression cassette comprising a nucleotide sequences selected from U6-1 to U6-8 according to SEQ ID Nos: 1 to 8 or sequences having a degree of sequence identity of at least 40%, as for example at least 50, 60, 70, 75, 80, 85, 90, 91, 92, 93, 94, 95, 96, 97, 98, or 99%, retaining their ability to functionally express bacterial tRNA^{Pyl} in insect cells, in particular Spodoptera cell lines, preferably Spodoptera frugiperda cell lines, like in particular Spodoptera frugiperda cell line Sf21 (DSMZ Nr. ACC119).
31. The vector of embodiment 29 or 30 wherein said tRNA^{Pyl} comprises a nucleotide sequence according to SEQ ID NO: 9 or a sequence having a degree of sequence identity of at least 70%, as for example at least 75, 80, 85, 90, 91, 92, 93, 94, 95, 96, 97, 98, or 99%, while retaining the tRNA^{Pyl} function.
32. The vector of anyone of the embodiments 19 to 31, wherein said bacterial O-RS synthetase is of archaebacterial origin, and preferably is PylRS comprising an amino acid sequence of SEQ ID NO: 27 or a sequence having a degree of sequence identity of at least 70%, as for example at least 75, 80, 85, 90, 91, 92, 93, 94, 95, 96, 97, 98, or 99%, while retaining the tRNA^{Pyl} function, in particular PylRS WT SEQ ID NO: 27 or PylRS AF SEQ ID NO: 30
33. An insect cell line or insect cell (ICL) capable of expressing one or more orthogonal bacterial aminoacyl tRNA synthetase/tRNA^{ncAA} O-RS/O-tRNA^{ncAA} pairs required for introducing at one or more ncAA residues into the amino acid sequence of a TP to be co-expressed by said ICL, wherein each bacterial tRNA^{ncAA} coding sequence is expressed under the control of an insect-cell derived regulatory sequence (RS^{IC}).
34. The ICL of embodiment 33, which is transiently or stably transfected with one or more vectors carrying the genetic information required for expressing said O-RS/O-tRNA^{ncAA} pairs.
35. The ICL of one of the embodiments 33 and 34, wherein said ICL is transfected with one or more baculoviral vectors carrying the genetic information required for expressing said TP and said one or more O-RS/O-tRNA^{ncAA} pairs.
36. The ICL of anyone of the embodiments 33 to 35, wherein said RS^{IC} and said ICL are derived from identical insect species.
37. The ICL one of the embodiments 33 or 36, wherein said RS^{IC} is derived from an insect of the genus Spodoptera, preferably Spodoptera frugiperda, like in particular Spodoptera frugiperda cell line Sf21 (DSMZ Nr. ACC119).
38. The ICL of one of the embodiments 33 to 37, wherein said RS^{IC} is selected from regulatory sequences recognized by (is functional for) RNA-polymerase III, in particular insect RNA-polymerase III.
39. The ICL of embodiment 38, wherein said RS^{IC} is selected from
   a) regulatory sequences of insect snRNA U6 genes, and
   b) regulatory sequences of insect tRNA genes, in particular H1 regulatory sequences; wherein embodiment a) is preferred.
40. The ICL of one of the embodiments 33 to 39, wherein said RS^{IC} comprises a U6 promoter sequence 5′-downstream of the insect snRNA U6 coding sequence, in particular 5′-downstream of the insect snRNA U6 coding sequence as depicted in FIG. 1, preferably 5′-downstream of the insect snRNA U6 coding sequence of SEQ ID NO:12.
41. The ICL of embodiment 40, wherein said U6 promoter is selected from nucleotide sequences corresponding to nucleotide residues
   a) 1 to 400 of SEQ ID NO. 1
   b) 1 to 392 of SEQ ID NO: 2
   c) 1 to 385 of SEQ ID NO: 3 to 8
   d) functional fragments thereof which retain the intended promoter activity, and in particular regulate the expression of tRNA^{ncAA} being under the control for said functional fragment, comprising a partial sequence of anyone of the nucleotide sequences as defined in a) to c).

As regards the provision of further functional fragments reference is made to embodiment 9, above.
42. The ICL of one of the embodiments 33 to 41, wherein said RS^{IC} comprises a U6 termination sequence 3′-upstream of the of insect snRNA U6 coding sequence.
43. The ILC of embodiment 42, wherein said U6 terminator is selected from nucleotide sequences corresponding to nucleotide residues
   a) 470 to 569 of SEQ ID NO:1
   b) 462 to 561 of SEQ ID NO:2
   c) 455 to 554 of SEQ ID NO:3 to 8
   d) functional fragments thereof, preferably which retain the intended terminator activity, and in particular regulate the expression of tRNA^{ncAA} being under the control for said functional fragment, comprising a partial sequence of anyone of the nucleotide sequences as defined in a) to c).

As regards the provision of further functional fragments reference is made to embodiment 11, above.
44. The ICL of anyone of the embodiments 33 to 43, comprising an expression cassette for bacterial, in particular archaebacterial, tRNA^{ncAA} which is construed according to the following scheme:
   5′-downstream U6 promoter/tRNA^{ncAA} coding sequence/3′-upstream U6 terminator
45. The ICL of anyone of the embodiments 33 to 44, wherein the bacterial tRNA^{ncAA} is of archaebacterial origin, in particular of a bacterium of the genus Methanosarcina, like M. hafniense, M. bakeri and M. mazei, preferably Methanosarcina mazei.
46. The ICL of anyone of the embodiments 33 to 45, wherein said bacterial tRNA^{ncAA} is Pyrrolysyl tRNA (tRNA^{Pyl}) from Methanosarcina mazei.
47. The ICL of anyone of the embodiments 33 to 46, wherein said bacterial tRNA^{Pyl} is encoded by an expression cassette comprising a nucleotide sequences selected from U6-1 to U6-8 according to SEQ ID Nos: 1 to 8 or sequences having a degree of sequence identity of at least 40%, as for example at lest 50, 60, 70, 75, 80, 85, 90, 91, 92, 93, 94, 95, 96, 97, 98, or 99%, retaining their ability to functionally express bacterial tRNA^{Pyl} in insect cells, in particular Spodoptera cell lines, preferably Spodoptera frugiperda cell lines, like in particular Spodoptera frugiperda cell line Sf21 (DSMZ Nr. ACC119).
48. The ICL of anyone of the embodiments 33 to 47, wherein said tRNA^{Pyl} comprises a nucleotide sequence according to SEQ ID NO: 9 or a sequence having a degree of sequence identity of at least 70%, as for example at lest 75, 80, 05, 90, 91, 92, 93, 94, 95, 96, 97, 98, or 99%, while retaining the tRNA^{Pyl} function.
49. The ICL of anyone of the embodiments 33 to 48, wherein said bacterial O-RS synthetase is of archaebacterial origin, and preferably is PylRS comprising an amino acid sequence of SEQ ID NO: 27 or a sequence having a degree of sequence identity of at least 70%, as for example at lest 75, 80, 05, 90, 91, 92, 93, 94, 95, 96, 97, 98, or 99%, while retaining the tRNA^{Pyl} function, in particular PylRS WT SEQ ID NO: 27 or PylRS AF SEQ ID NO: 30.
50. The ICL of anyone of the embodiments 33 to 49, wherein said ICL is selected from Spodoptera cell lines, in particular Spodoptera frugiperda cell lines, preferably Sf21 (DSMZ Nr. ACC119); or Drosophila cell lines in particular Drosophila melanogaster cell lines preferably Schneider-2 R+ (Drosophila Genomics Research Center (DGRC) stock number 150). Preferably, said ICL is selected from Spodoptera cell lines, more preferably Spodoptera frugiperda cell lines, most preferably Sf21 (DSMZ Nr. ACC119). Further said ILC may be selected from Trichoplusia cell lines, preferably Trichoplusia ni BTI-Tn-5B1-4 (High Five, Invitrogen).
51. The ICL of anyone of the embodiments 33 to 50, transfected with a baculovirus vector.
52. The ICL of embodiment 51, wherein said vector is as defined in anyone of the embodiments 19 to 32.
53. An expression cassette encoding tRNA^{Pyl} selected from U6-1 to U6-8 according to SEQ ID Nos: 1 to 8 or sequences having a degree of sequence identity of at least 40% as for example at lest 50, 60, 70, 75, 80, 85, 90, 91, 92, 93, 94, 95, 96, 97, 98, or 99%, retaining their ability to functionally express bacterial tRNA^{Pyl} in insect cells.
54. A (engineered) target protein (TP) comprising in its amino acid sequence one or more identical or different non-canonical amino acid (ncAA) residues, obtained by expressing the TP encoding nucleotide sequence (CS^{TP}) in an insect cell line (ICL), in the presence of said one or more ncAAs, wherein said CS^{TP}) comprises one or more selector codons encoding said one or more ncAA residues.
55. The TP of embodiment 54, obtained by a method of anyone of the embodiments 1 to 13, optionally further chemically modified by performing a click reaction with said at least one non-canonical amino acid (ncAA) residue contained in its amino acid sequence.
56. The TP of embodiment 55, wherein said ncAA contains a clickable functionalized side chain, such as PrK, SCO, TCO, TCO*, BOC (butoxycarbonyl lysine) and derivatives.
57. The TP of anyone of the embodiments 54 to 56, selected from engineered prokaryotic or in particular eukaryotic, polypeptides, proteins or enzymes, like in particular immunoglobulin molecules, fluorophores, cellular marker proteins and transcription factors.
58. A kit for preparing a recombinant target polypeptide TP having one or more ncAA residues, comprising at least one ncAA or salt thereof and further comprising at least one baculoviral vector of one of the embodiments 19 to 32.
59. A promoter sequence is selected from nucleotide sequences corresponding to nucleotide residues
   a) 1 to 400 of SEQ ID NO. 1
   b) 1 to 392 of SEQ ID NO: 2
   c) 1 to 385 of SEQ ID NO: 3 to 8
   d) functional fragments thereof which retain the intended promoter activity, and in particular regulate the expression of tRNA^{ncAA} being under the control for said functional fragment, comprising a partial sequence of anyone of the nucleotide sequences as defined in a) to c).

As regards the provision of further functional fragments reference is made to embodiment 9, above.

For example suitable functional fragments of promoters may comprise less than 400 consecutive nucleotide residues, less than 300, lest than 250, less than 200, less than 150, or less than 100 and at least 20, at least 30, at least 40, at least 50, at least 60, at least 70 or at least 80 or at least 90 nucleotide residues preferably 5′-downstream of the insect snRNA U6 coding sequence of SEQ ID NO:12, which may be a single fragment of consecutive 5′-downstream residues or may encompass more than one partial sequence comprising one or more functional partial elements, required for the intended promoter activity. As non-limiting examples of such functional partial elements there may be mentioned TATA boxes or PSEA elements as described in the art.

For example suitable functional fragments of promoters may have a sequence length of 20, 30, 40 or 50 to 399, like 20 to 350, 20 to 300, 20 to 250, 20 to 200, 20 to 150, 20 to 100, or like 30 to 350, 30 to 300, 30 to 250, 30 to 200, 30 to 150, 30 to 100, or like 40 to 350, 40 to 300, 40 to 250, 40 to 200, 40 to 150, 40 to 100, or like 50 to 350, 50 to 300, 50 to 250, 50 to 200, 50 to 150, 50 to 100 nucleotide residues.

According to a very particular embodiment of the above methods, cell lines, vectors and expression cassettes the following preferred meanings apply individually or in combination:
said ICL is Sf21 (DSMZ Nr. ACC119),
said ICL is stably transfected with 1 or 2 baculoviral vectors carrying the genetic information (coding sequences and regulatory sequences) required for expressing said TP and said one or more O-RS/O-tRNA^{ncAA} pairs;
said tRNA^{ncAA} is pyrrolysyl tRNA (tRNA^{Pyl}) from Methanosarcina mazei; and
said RS^{IC} comprises a U6 termination sequence 3′-upstream of the of insect snRNA U6 coding sequence, in particular 3′-upstream of the insect snRNA U6 coding sequence as depicted in FIG. 1, preferably 3′-upstream of the insect snRNA U6 coding sequence of SEQ ID NO:12; most preferably selected from nucleotide sequences corresponding to consecutive nucleotide residues 1 to 400 of SEQ ID NO. 1, 1 to 392 of SEQ ID NO: 2 or 1 to 385 of SEQ ID NO: 3 to 8.

### C. Further Embodiments of the Invention

1. Enzymes, Target Polypeptides (TP) and Functional Equivalents and Mutants Thereof

The present invention is not limited to the particular proteins or enzymes (like aminoacyl-tRNA synthetases, target polypeptides) concretely disclosed or described herein, but rather also extends to functional equivalents or analogs thereof.

“Functional equivalents” or analogs of the concretely disclosed enzymes or target polypeptides are within the scope of the present invention. Such functional equivalents furthermore possess the desired biological activity, as for example tRNA synthetase activity.

For example “functional equivalents” are understood to include enzymes and mutants that have an at least 1%, in particular at least about 5 to 10%, for example at least 10% or at least 20%, for example at least 50% or 75% or 90% higher or lower activity of an enzyme, comprising an amino acid sequence concretely defined herein.

The activity information for “functional equivalents” refers herein, unless stated otherwise, to activity determinations, performed by means of a reference substrate (for example particular ncAA) under standardized conditions which easily may be defined by a skilled reader.

“Functional equivalents” may, moreover, be stable e.g. between pH 4 to 11 and advantageously possess a pH optimum in a range from pH 5 to 10, such as in particular 6.5 to 9.5 or 7 to 8 or at about 7.5, and a temperature optimum in the range from 15° C. to 80° C. or 20° C. to 70° C., for example about 30 to 60° C. or about 35 to 45° C., such as at 40° C.

“Functional equivalents” are to be understood according to the invention to include in particular also “mutants”, which have in at least one sequence position of the particular amino acid sequences, an amino acid other than that concretely stated, but nevertheless possess one of the aforementioned biological activities.

“Functional equivalents” comprise the mutants obtainable by one or more, for example 1 to 50, 2 to 30, 2 to 15, 4 to 12 or 5 to 10 “additional mutations”, such as amino acid additions, substitutions, deletions and/or inversions, wherein the stated changes can occur in any sequence position, provided they lead to a mutant with the property profile according to the invention. Functional equivalence is in particular also present when the reactivity profiles between mutant and unaltered polypeptide coincide qualitatively, i.e. for example the same substrates are used at a different rate.

Nonlimiting examples of suitable amino acid substitutions are given in the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also “precursors” of the polypeptides described as well as “functional derivatives” and “salts” of the polypeptides.

“Precursors” are natural or synthetic precursors of the polypeptides with or without the desired biological activity.

The term “salts” means both salts of carboxyl groups and salts of acid addition of amino groups of the protein molecules according to the invention. Salts of carboxyl groups can be produced in a manner known per se and comprise inorganic salts, for example sodium, calcium, ammonium, iron and zinc salts, and salts with organic bases, for example amines, such as triethanolamine, arginine, lysine, piperidine and the like. Salts of acid addition, for example salts with mineral acids, such as hydrochloric acid or sulfuric acid and salts with organic acids, such as acetic acid and oxalic acid, are also objects of the invention.

“Functional derivatives” of polypeptides according to the invention can also be produced on functional amino acid side groups or at their N- or C-terminal end by known techniques. Derivatives of this kind comprise for example aliphatic esters of carboxylic acid groups, amides of carboxylic acid groups, obtainable by reaction with ammonia or with a primary or secondary amine; N-acyl derivatives of free amino groups, produced by reaction with acyl groups; or O-acyl derivatives of free hydroxyl groups, produced by reaction with acyl groups.

“Functional equivalents” naturally also comprise polypeptides that are accessible from other organisms, and naturally occurring variants thereof. For example areas of homologous sequence regions can be established by sequence comparison and equivalent enzymes can be determined based on the concrete information of the invention.

“Functional equivalents” also comprise fragments, preferably individual domains or sequence motifs, of the polypeptides according to the invention, which for example have the desired biological function.

“Functional equivalents” are moreover fusion proteins, which have one of the aforementioned polypeptide sequences or functional equivalents derived therefrom and at least one further, functionally different therefrom, heterologous sequence in functional N- or C-terminal linkage (i.e. without mutual substantial functional impairment of the fusion protein parts). Nonlimiting examples of heterologous sequences of this kind are e.g. signal peptides, histidine anchors or enzymes.

“Functional equivalents” that are also included according to the invention are homologs to the concretely disclosed proteins. These possess at least 60%, preferably at least 75%, especially at least 85%, for example 90, 91, 92, 93, 94, 95, 96, 97, 98 or 99%, homology (or identity) to one of the concretely disclosed amino acid sequences, calculated using the algorithm of Pearson and Lipman, Proc. Natl. Acad. Sci. (USA) 85(8), 1988, 2444-2448. A percentage homology or identity of a homologous polypeptide according to the invention means in particular percentage identity of the amino acid residues relative to the total length of one of the amino acid sequences concretely described herein.

The percentage identity values can also be determined on the basis of BLAST alignments, blastp algorithms (protein-protein BLAST), or using the Clustal settings given below.

In the case of a possible protein glycosylation, “functional equivalents” according to the invention comprise proteins of the type designated above in deglycosylated or glycosylated form as well as modified forms obtainable by changing the glycosylation pattern.

Homologs of the proteins or polypeptides used or prepared according to the invention can be produced by mutagenesis, e.g. by point mutation, lengthening or shortening of the protein.

Homologs of the proteins according to the invention can be identified by screening combinatorial databases of mutants, for example shortened mutants. For example a variegated database of protein variants can be produced by combinatorial mutagenesis at nucleic acid level, for example by enzymatic ligation of a mixture of synthetic oligonucleotides. There are a great many methods that can be used for producing databases of potential homologs from a degenerated oligonucleotide sequence. The chemical synthesis of a degenerated gene sequence can be carried out in an automatic DNA synthesizer, and the synthetic gene can then be ligated into a suitable expression vector. The use of a degenerated set of genes makes it possible to provide all sequences, in one mixture, which code for the desired set of potential protein sequences. Methods for the synthesis of degenerated oligonucleotides are known by a person skilled in the art (e.g. Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

Several techniques for screening gene products of combinatorial databases, which were produced by point mutations or shortening, and for screening cDNA databases for gene products with a chosen property, are known in the prior art. These techniques can be adapted for rapid screening of gene banks that have been produced by combinatorial mutagenesis of homologs according to the invention. The techniques used most often for screening large gene banks, as the basis for high-throughput analysis, comprise cloning the gene bank into replicatable expression vectors, transforming suitable cells with the resultant vector bank and expressing the combinatorial genes in conditions in which detection of the desired activity facilitates the isolation of the vector that codes for the gene whose product was detected. Recursive ensemble mutagenesis (REM), a technique that increases the frequency of functional mutants in the databases, can be used in combination with the screening tests, to identify homologs (Arkin and Yourvan (1992) PNAS 89:7811-7815; Delgrave et al. (1993) Protein Engineering 6(3):327-331).

2. Nucleic Acids and Constructs

2.1 Nucleic Acids and Functional Equivalents Thereof

The invention also relates to regulatory nucleic acid sequences (like promoter and terminator sequences), and nucleic acid sequences that code for enzymes or target polypeptides or tRNAs as described above, or mutants or functional equivalents thereof.

The present invention also relates to nucleotide sequences/nucleic acids with a specified degree of identity to the concrete sequences described herein.

“Identity” between two nucleic acids means identity of the nucleotides in each case over the whole length of nucleic acid, in particular the identity that is calculated by comparison by means of the Vector NTI Suite 7.1 software from the company Informax (USA) using the Clustal method (Higgins D G, Sharp P M. Fast and sensitive multiple sequence alignments on a microcomputer. Comput Appl. Biosci. 1989 April; 5(2):151-1), setting the following parameters:

| Multiple alignment parameters: | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighting | 0 |
| Pairwise alignment parameter: | |
| FAST algorithm | on |
| K-tuplesize | 1 |
| Gap penalty | 3 |
| Window size | 5 |
| Number of best diagonals | 5 |

As an alternative, the identity can also be determined according to Chenna, Ramu, Sugawara, Hideaki, Koike, Tadashi, Lopez, Rodrigo, Gibson, Toby J, Higgins, Desmond G, Thompson, Julie D. Multiple sequence alignment with the Clustal series of programs. (2003) Nucleic Acids Res 31 (13):3497-500, according to Internet address: http://www.ebi.ac.uk/Tools/clustalw/index.html# and with the following parameters:

| | |
|---|---|
| DNA Gap Open Penalty | 15.0 |
| DNA Gap Extension Penalty | 6.66 |
| DNA Matrix | Identity |
| Protein Gap Open Penalty | 10.0 |
| Protein Gap Extension Penalty | 0.2 |
| Protein matrix | Gonnet |
| Protein/DNA ENDGAP | −1 |
| Protein/DNA GAPDIST | 4 |

All nucleic acid sequences mentioned herein (single-stranded and double-stranded DNA and RNA sequences, for example cDNA, mRNA, tRNA) can be produced in a manner known per se by chemical synthesis from the nucleotide building blocks, for example by fragment condensation of individual overlapping, complementary nucleic acid building blocks of the double helix. The chemical synthesis of oligonucleotides can for example be carried out in a known manner, by the phosphoroamidite technique (Voet, Voet, 2nd edition, Wiley Press New York, pages 896-897). The adding-on of synthetic oligonucleotides and filling of gaps using the Klenow fragment of DNA polymerase and ligation reactions as well as general cloning techniques are described in Sambrook et al. (1989), Molecular Cloning: A Laboratory Manual, Cold Spring Harbor Laboratory Press.

The invention also relates to nucleotide sequences (single-stranded and double-stranded DNA and RNA sequences, for example cDNA, mRNA), coding for one of the above polypeptides, enzymes or tRNAs and functional equivalents thereof, which are accessible e.g. using artificial nucleotide analogs.

The invention relates both to isolated nucleic acid molecules, which code for polypeptides, enzymes or tRNAs according to the invention or biologically active segments thereof, and to nucleic acid fragments, which can be used for example as hybridization probes or primers for the identification or amplification of coding nucleic acids according to the invention.

The nucleic acid molecules according to the invention can in addition contain untranslated sequences of the 3′- and/or 5′-end of the coding gene region.

The invention further comprises the nucleic acid molecules complementary to the concretely described nucleotide sequences, or a segment thereof.

The nucleotide sequences according to the invention make it possible to produce probes and primers that can be used for the identification and/or cloning of homologous sequences in other cell types and organisms. Said probes or primers usually comprise a nucleotide sequence region which hybridizes under “stringent” conditions (see below) to at least about 12, preferably at least about 25, for example about 40, 50 or 75 successive nucleotides of a sense strand of a nucleic acid sequence according to the invention or of a corresponding antisense strand.

An “isolated” nucleic acid molecule is separated from other nucleic acid molecules that are present in the natural source of the nucleic acid, and moreover can be essentially free of other cellular material or culture medium, when it is produced by recombinant techniques, or free of chemical precursors or other chemicals, when it is chemically synthesized.

A nucleic acid molecule according to the invention can be isolated by standard techniques of molecular biology and the sequence information provided according to the invention. For example, cDNA can be isolated from a suitable cDNA-bank, using one of the concretely disclosed complete sequences or a segment thereof as hybridization probe and standard hybridization techniques (as described for example in Sambrook, J., Fritsch, E. F. and Maniatis, T. Molecular Cloning: A Laboratory Manual. 2nd edition, Cold Spring Harbor Laboratory, Cold Spring Harbor Laboratory Press, Cold Spring Harbor, N.Y., 1989). Moreover, a nucleic acid molecule, comprising one of the disclosed sequences or a segment thereof, can be isolated by polymerase chain reaction, using the oligonucleotide primers that were constructed on the basis of this sequence. The nucleic acid thus amplified can be cloned into a suitable vector and can be characterized by DNA sequence analysis. The oligonucleotides according to the invention can moreover be produced by standard methods of synthesis, e.g. with an automatic DNA synthesizer.

Nucleotide sequences according to the invention or derivatives thereof, homologs or parts of these sequences, can be isolated for example with usual hybridization methods or PCR techniques from other pro- or eukaryotic organisms, like insects, bacteria, archaebacteria, e.g. via genomic or cDNA databases. These DNA sequences hybridize under standard conditions to the sequences according to the invention.

“Hybridization” means the capacity of a poly- or oligonucleotide to bind to an almost complementary sequence under standard conditions, whereas under these conditions nonspecific binding between noncomplementary partners does not occur. For this, the sequences can be up to 90-100% complementary. The property of complementary sequences of being able to bind specifically to one another is utilized for example in Northern or Southern blotting or in primer binding in PCR or RT-PCR.

Short oligonucleotides of the conserved regions are used advantageously for hybridization. However, longer fragments of the nucleic acids according to the invention or the complete sequences can also be used for hybridization. These standard conditions vary depending on the nucleic acid used (oligonucleotide, longer fragment or complete sequence) or depending on which type of nucleic acid, DNA or RNA, is used for hybridization. Thus, for example, the melting temperatures for DNA:DNA hybrids are approx. 10° C. lower than those of DNA:RNA hybrids of the same length.

Standard conditions mean for example, depending on the nucleic acid, temperatures between 42 and 58° C. in an aqueous buffer solution with a concentration between 0.1 to 5×SSC (1×SSC=0.15 M NaCl, 15 mM sodium citrate, pH 7.2) or additionally in the presence of 50% formamide, for example 42° C. in 5×SSC, 50% formamide. Advantageously, the hybridization conditions for DNA:DNA hybrids are 0.1×SSC and temperatures between about 20° C. to 45° C., preferably between about 30° C. to 45° C. For DNA:RNA hybrids the hybridization conditions are advantageously 0.1×SSC and temperatures between about 30° C. to 55° C., preferably between about 45° C. to 55° C. These stated temperatures for hybridization are for example calculated melting temperature values for a nucleic acid with a length of approx. 100 nucleotides and a G+C content of 50% in the absence of formamide. The experimental conditions for DNA hybridization are described in relevant textbooks on genetics, for example Sambrook et al., “Molecular Cloning”, Cold Spring Harbor Laboratory, 1989, and can be calculated using formulas known by a person skilled in the art, for example depending on the length of the nucleic acids, the type of hybrids or the G+C content. Further information on hybridization can be obtained by a person skilled in the art from the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

“Hybridization” can in particular take place under stringent conditions. Said hybridization conditions are described for example by Sambrook, J., Fritsch, E. F., Maniatis, T. in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6.

“Stringent” hybridization conditions mean in particular: Incubation at 42° C. overnight in a solution consisting of 50% formamide, 5×SSC (750 mM NaCl, 75 mM trisodium citrate), 50 mM sodium phosphate (pH7.6), 5× Denhardt solution, 10% dextran sulfate and 20 g/ml denatured, sheared salmon sperm DNA, followed by a step of washing the filters with 0.1×SSC at 65° C.

The invention also relates to derivatives of the concretely disclosed or derivable nucleotide sequences.

Thus, further nucleotide sequences according to the invention can be derived from particular sequences as referred to herein and differ from them by addition, substitution, insertion or deletion of single or several, as for example 1 to 50, 2 to 30, 2 to 15, 4 to 12 or 5 to 10 nucleotides, but furthermore code for polypeptides, Enzyme or tRNA with the desired property profile. as for example 1 to 50, 2 to 30, 2 to 15, 4 to 12 or 5 to 10

The invention also includes nucleotide sequences that comprise so-called silent mutations, as for example 1 to 50, 2 to 30, 2 to 15, 4 to 12 or 5 to 10, or are altered corresponding to the codon-usage of a special original or host organism, compared with a concretely stated sequence, as well as naturally occurring variants, for example splice variants or allele variants, thereof.

It also relates to sequences obtainable by conservative nucleotide substitutions, as for example 1 to 50, 2 to 30, 2 to 15, 4 to 12 or 5 to 10, (i.e. as a result thereof the corresponding amino acid in question is replaced with an amino acid of the same charge, size, polarity and/or solubility).

The invention also relates to the molecules derived by sequence polymorphisms from the concretely disclosed nucleic acids. These genetic polymorphisms can exist between individuals within a population owing to natural variation. These natural variations usually bring about a variance of 1 to 5% in the nucleotide sequence of a gene.

Derivatives of the nucleotide sequences according to the invention include for example allele variants that have at least 60% homology at the derived amino acid level, preferably at least 80% homology, quite especially preferably at least 90% homology over the whole sequence region (regarding homology at the amino acid level, reference should be made to the above account relating to polypeptides). The homologies can advantageously be higher over partial regions of the sequences.

Furthermore, derivatives also mean homologs of the nucleotide sequences according to the invention, for example fungal or bacterial, mammalian or insect homologs, shortened sequences, single-strand DNA or RNA of the coding and noncoding DNA sequence.

The regulatory sequences of the invention, like promoters or terminator sequences can be altered by at least one nucleotide exchange, at least one, as for example 1 to 50, 2 to 30, 2 to 15, 4 to 12 or 5 to 10, insertion, inversion and/or deletion, without the functionality or efficacy of the promoters being impaired.

2.2 Generation of Functional Mutants

Furthermore, methods for producing functional mutants of enzymes or target polypeptides according to the invention are known by a person skilled in the art.

Depending on the technology used, a person skilled in the art can introduce completely random or even more-directed mutations in genes or also noncoding nucleic acid regions (which for example are important for the regulation of expression) and then prepare gene libraries. The necessary methods of molecular biology are known by a person skilled in the art and for example are described in Sambrook and Russell, Molecular Cloning. 3^{rd} edition, Cold Spring Harbor Laboratory Press 2001.

Methods for altering genes and therefore for altering the proteins that they encode have long been familiar to a person skilled in the art, for example

site-directed mutagenesis, in which single or several nucleotides of a gene are deliberately exchanged (Trower M K (Ed.) 1996; In vitro mutagenesis protocols. Humana Press, New Jersey),

saturation mutagenesis, in which a codon for any amino acid can be exchanged or added at any point of a gene (Kegler-Ebo D M, Docktor C M, DiMaio D (1994) Nucleic Acids Res 22:1593; Barettino D, Feigenbutz M, Valcárel R, Stunnenberg HG (1994) Nucleic Acids Res 22:541; Barik S (1995) Mol Biotechnol 3:1),

the error-prone polymerase chain reaction (error-prone PCR), in which nucleotide sequences are mutated by error-prone DNA polymerases (Eckert K A, Kunkel T A (1990) Nucleic Acids Res 18:3739);

the SeSaM method (sequence saturation method), in which preferred exchanges are prevented by the polymerase. Schenk et al., Biospektrum, Vol. 3, 2006, 277-279

the passaging of genes in mutator strains, in which, for example owing to defective DNA repair mechanisms, there is an increased mutation rate of nucleotide sequences (Greener A, Callahan M, Jerpseth B (1996) An efficient random mutagenesis technique using an E. coli mutator strain. In: Trower M K (Ed.) In vitro mutagenesis protocols. Humana Press, New Jersey), or

DNA shuffling, in which a pool of closely related genes is formed and digested and the fragments are used as templates for a polymerase chain reaction, in which, by repeated strand separation and bringing together again, finally mosaic genes of full length are produced (Stemmer WPC (1994) Nature 370:389; Stemmer W P C (1994) Proc Natl Acad Sci USA 91:10747).

Using so-called directed evolution (described for instance in Reetz M T and Jaeger K-E (1999), Topics Curr Chem 200:31; Zhao H, Moore J C, Volkov A A, Arnold F H (1999), Methods for optimizing industrial enzymes by directed evolution, in: Demain A L, Davies J E (Ed.) Manual of industrial microbiology and biotechnology. American Society for Microbiology), a person skilled in the art can produce functional mutants in a directed manner and on a large scale. For this, in a first step, gene libraries of the respective proteins are first produced, for example using the methods given above. The gene libraries are expressed in a suitable way, for example by bacteria or by phage display systems.

The relevant genes of host organisms that express functional mutants with properties that largely correspond to the desired properties can be submitted to another round of mutation. The steps of mutation and selection or screening can be repeated iteratively until the present functional mutants have the desired properties to a sufficient extent. Using this iterative procedure, a limited number of mutations, for example 1, 2, 3, 4 or 5 mutations, can be effected in stages and can be assessed and selected for their influence on the enzyme property in question. The selected mutant can then be submitted to a further mutation step in the same way. In this way the number of individual mutants to be investigated can be reduced significantly.

The results according to the invention also provide important information relating to structure and sequence of the relevant enzymes, which is required for deliberately generating further enzymes or target polypeptides with desired modified properties. In particular so-called “hot spots” can be defined, i.e. sequence segments that are potentially suitable for modifying an enzyme property by introducing targeted mutations.

Information can also be deduced regarding amino acid sequence positions, in the region of which mutations can be carried out that should probably have little effect on enzyme activity, and can be designated as potential “silent mutations”.

2.3 Nucleic Acid Constructs

The invention further relates to, in particular recombinant, expression constructs or expression cassettes, containing, under the genetic control of regulatory nucleic acid sequences as defined herein, a nucleic acid sequence coding for a polypeptide, enzyme or tRNA. The invention also relates to, in particular recombinant, vectors, comprising at least one of these expression constructs.

An “expression unit” means, according to the invention, a nucleic acid with expression activity, which comprises a promoter, as defined herein, and after functional linkage with a nucleic acid to be expressed or a gene, regulates the expression, i.e. the transcription and the translation of said nucleic acid or said gene. Therefore in this connection it is also called a “regulatory nucleic acid sequence”. In addition to the promoter, other regulatory elements, for example enhancers, can also be present.

An “expression cassette” or “expression construct” means, according to the invention, an expression unit that is functionally linked to the nucleic acid to be expressed or the gene to be expressed. In contrast to an expression unit, an expression cassette therefore comprises not only nucleic acid sequences that regulate transcription and translation, but also the nucleic acid sequences that are to be expressed as protein or tRNA as a result of the transcription and translation.

The terms “expression” or “overexpression” describe, in the context of the invention, the production or increase in intracellular activity of one or more enzymes in a microorganism or other cells, like insect cells as described herein, which are encoded by the corresponding DNA. For this, it is possible for example to introduce a gene into an organism, replace an existing gene with another gene, increase the copy number of the gene or genes, use a strong promoter or use a gene that codes for a corresponding enzyme with a high activity; optionally, these measures can be combined.

Preferably said constructs according to the invention comprise a promoter 5′-upstream of the respective coding sequence and a terminator sequence 3′-downstream and optionally other usual regulatory elements, in each case operatively linked with the coding sequence.

A “promoter”, or a “nucleic acid with promoter activity” or of a “promoter sequence” means, according to the invention, a nucleic acid which, functionally linked to a nucleic acid to be transcribed, regulates the transcription of said nucleic acid.

A “functional” or “operative” linkage means, in this connection, for example the sequential arrangement of one of the nucleic acids with promoter activity and of a nucleic acid sequence to be transcribed and optionally further regulatory elements, for example nucleic acid sequences that ensure the transcription of nucleic acids, and for example a terminator, in such a way that each of the regulatory elements can perform its function during transcription of the nucleic acid sequence. This does not necessarily require a direct linkage in the chemical sense. Genetic control sequences, for example enhancer sequences, can even exert their function on the target sequence from more remote positions or even from other DNA molecules. Arrangements are preferred in which the nucleic acid sequence to be transcribed is positioned behind (i.e. at the 3′-end of) the promoter sequence, so that the two sequences are joined together covalently. The distance between the promoter sequence and the nucleic acid sequence to be expressed can be smaller than 200 base pairs, or smaller than 100 base pairs or smaller than 50 base pairs.

In addition to promoters and terminator, the following may be mentioned as examples of other regulatory elements: targeting sequences, enhancers, polyadenylation signals, selectable markers, amplification signals, replication origins and the like. Suitable regulatory sequences are described for example in Goeddel, Gene Expression Technology: Methods in Enzymology 185, Academic Press, San Diego, Calif. (1990).

Nucleic acid constructs according to the invention comprise in particular SEQ ID NOs: 1 to 8 comprising the coding sequence of tRNApyl which has been linked operatively or functionally with one or more regulatory signals advantageously for controlling, e.g. increasing, gene expression; and the nucleic acid sequences derivable therefrom.

Particular U6-tRNA^{Pyl}-3term sequences (SEQ ID NOs: 1 to 8) are depicted below:

Promoter sequence in italics

tRNA sequence underlined

3′termination signal double underlined

U6-1(Sf21)-rRNA^{Pyl}-3term (U6-1)
(SEQ ID NO: 1)
Ataagttgagttatggcttaaaaaaaattattttttttctatttcatactgttaaaaatcaacgcaatttacaatctgggaaatgaaatatc
caataattaagttagggttacgaagtaattggaatatcgattcaattgtaatcgatttacggtacagagttcatactatttacgaaaatgct
ttaagtatttctatgatgatcggatgatttatttaattaaaataataaaatctattagaattacagtattcagagttaaaactaaataatta
tctacataattaatataagtcgattcacatttactcatcgattattatatttttaatctgtgcaactctgacttgacattgacatgcaatca
atgacatcgatcggcaccaagtatatgtttggaaacctgatcatgtagatcgaatggactctaaatccgttcagccgggttagattcccggg
gtttccgttttttgtaatcgtagatacaatgtcgaagattctggcccgtttattgtacatgtgtcccgcgttgctgactccccaaattctgg
tgttagtttaaaacc
U6-2(Sf21)-tRNA^{Pyl}-3term (U6-2)
(SEQ ID NO: 2)
Acttaactactcaaaaagtgagggccagcagctcgaccaatgtaaaaccttgcgaggtgcgaggttaccggggacccaatcaaagagtataa
taactatagggaaaggcccaaccccccccccccccactgtatgtaaaaatataagacctatttctcaacctataaacctatgcaataaaaca
tccactagattagtctagtgactagactagaccattgttagttaacagtagttcggctagatggcgccaaattggttcttttagtgaacggt
agatggcgctgtactcaatcttcatacaaatcatgttaaatgtatgggattctacatcgcgctatcaaagttttcattgtgtttgtgaaggg
tacaataattttgccttggcaagtggaaacctgatcatgtagatcgaatggactctaaatccgttcagccgggttagattcccggggtttcc
gttttttgaagagtttcagtttggtatggtttttctattttcaaattggtatgagggagtaagcataatcaaatttaatttcttttgttaaa
ctttagctt
U6-3(Sf21)-tRNA^{Pyl}-3term (U6-3)
(SEQ ID NO: 3)
Agatctacgaattgttatttcgactttaatttttattaaactacgtaattattgttttatttttcaatgagtttcgtattacaaattgttct
aatgtttacctacatgtttaaaagatttcggcactgatcaaaatgtattcataccttacatactacccaatcaaaggctttacaagttactt
tcggcacatcgtctgtcaatgccataacttctgcagaaaatgggtcgagtttcggcctttcgcatcctttgcctttctcttgtaaacagtac
ttcatggcgcggttttcaactatactgtaaagtaattaaagtaattacctacataattgtatgattggactaccttgagtgacttggactaa
gatcttggactaagatcggaaacctgatcatgtagatcgaatggactctaaatccgttcagccgggttagattcccggggtttccgtttttt
ataatattaataagttatggaagaacggtgtctcccaatacaggctgtctatgcttaacgggaggctccaatcacaatcttttttgtacaat
cc
U6-4(Sf21)-tRNA^{Pyl}-3term (U6-4)
(SEQ ID NO: 4)
Ttatgcgagtgaggttaccggaggttcaattacccccttacactgtgtgtaaaatagataacctttttctcaacctaaactcaaactcaaat
catttattgcattcatgtgtacatttagatgatacataattaggagtatacctagtatacctagtataaacacatgaataatagactagcta
gagtctagtagtgtctacaccagactatttttagttaacagtagtttaactagatggcgctaaattagttcttttagtaaacggtagatggc
gctgtacttaatcgtcatacaaatcatgccgaatgtatgagattctacatcgcgctatcaaagtttttattgtgtttgtgagcggtacaata
attttgccatagcaagtggaaacctgatcatgtagatcgaatggactctaaatccgttcagccgggttagattcccggggtttccgtttttt
gaagagtttcagtttggtatggtttttctattttcaaattggtatgaggaaataagcataatcaaatttaatttcttttgtaaaactttagc
tt
U6-5(Sf21)-tRNA^{Pyl}-3term (U6-5)
(SEQ ID NO: 5)
agaaataaaattgaaatattcgatcaagttcaattttatgtctactgagatagttgatatagcatacctaccggtaaatttctacgttaaaa
aaaacaaaacagaaaatatgtcattcattattttcggtatttagtagcttttaataaataatttcaacataaaaatatacaaaaagaaatta
ttcatattaatttctaattttcaacttaaagatcccgtacagtttgacaaccattaaattaacttatttcttaaagtttaccaacagatggc
gttgtactcaacccacatacaaattgcgtcaaatgtatgggattctacatcgcgctatgaaagttttcattgtgtttgtgagcggtacaata
attttgccttagcaagtggaaacctgatcatgtagatcgaatggactctaaatccgttcagccgggttagattcccggggtttccgtttttt
gaagaaattttaaataaaaaaaattgttttattttatttttttaagtattctctattacataattctatacgtaggtatttgtcattctatg
cg
U6-6(Sf21)-tRNA^{Pyl}-3term (U6-6)
(SEQ ID NO: 6)
Ttgaaaatcgggttaaaatatacaatatcaacgacatctatcgttcatattcagaaacggattacgagttaactagcgccatctgttgttgt
gtaagtaacaacactgatatacttgtgtggaatagttccgacagaatttgtagatggcgctgtaataaaaatattatttaaaaacatgtatt
tttcacaattttatatattattgtaagatatttcgtgatattttataataaaaaatacattaatagtaaatattgtaattaaaaaaaggttt
caccttatttcattaaagattttaagaaatataacatgaaactctaaatcgcgatatcaacatttttgttgtttggtgcctaatatacaaaa
attcgtgctcgaccaccggaaacctgatcatgtagatcgaatggactctaaatccgttcagccgggttagattcccggggtttccgtttttt
gaagagtttcagtacgtttataattttattattatttatttatagtaaaaacgtgactaataaacaaagacgattgtttatttgtatgcaat
tt
U6-7(Sf21)-tRNA^{Pyl}-3term (U6-7)
(SEQ ID NO: 7)
Tcggttcaaaatatacaataccaacgacatctgtagttcatattcagaaacgtgtcacgggttaactagcgccatctattgttgtgtaagta
atattgataaaacgatgccatactgtgcggaaaagttccgacagaatttatagatggcgctgtaataaaaatattatttaagaacatgtatt
tttcaaaattttatatattattgtaagatatttcatgatattttataataaaaaatatgttaatagtaaatattgtaattaaaagtgggttt
gaccttatttcattgaaaatttaaagaaatataaaacaaaactctaaatcgcgatatcaacatttttgttgttcggtgcctaatgtactaaa
attcgtgctttacaaccggaaacctgatcatgtagatcgaatggactctaaatccgttcagccgggttagattcccggggtttccgtttttt
gaagaatgtcgctaagatagaattttaataattctttatttttggtaaatccgtgactaaaaacaaaagtgattgtttatttttttaactta
ag
U6-8(Sf21)-tRNA^{Pyl}-3term (U6-8)
(SEQ ID NO: 8)
attgtttattttttataaaagctgatatataaataaatattaactgataaataaaaaaatactttcttggaacaattgaagggaataatgat
gaaaaattttgctacgtgtaaaaaaaggactttagttcttttacgtttcgttagatggcgctttttacaaagtacgactaccaagtttaatt
ttattcattaaaaatagaaaattagtagaatttgtaaatttattctacaaaaaaatataaataaagtctgaaattttactatacataatttt
tcaatccaaaatcaattactatcatccagtaatttacaaaatctctgcatcgcgctagtaaaatttttatgctaagaatcatgtataccaaa
acggttattccacaagtggaaacctgatcatgtagatcgaatggactctaaatccgttcagccgggttagattcccggggtttccgtttttt
ggacattttcattttggtgaatattttaaaaatgctttgtatttcatcacatcttttattacatttctttcatcacatcacagtgatttttt
tt

In addition to these regulatory sequences, the natural regulation of these sequences can still be present before the actual structural genes and optionally can have been genetically altered, so that the natural regulation has been switched off and expression of the genes has been increased. The nucleic acid construct can, however, also be of simpler construction, i.e. no additional regulatory signals have been inserted before the coding sequence and the natural promoter, with its regulation, has not been removed. Instead, the natural regulatory sequence is mutated so that regulation no longer takes place and gene expression is increased.

A particular nucleic acid construct also may contain one or more of the “enhancer” sequences, functionally linked to the promoter, which make increased expression of the nucleic acid sequence possible. Additional advantageous sequences can also be inserted at the 3′-end of the DNA sequences, such as further regulatory elements or terminators. One or more copies of the nucleic acids according to the invention can be contained in the construct. The construct can also contain other markers, such as antibiotic resistances or auxotrophy complementing genes, optionally for selection on the construct.

Examples of suitable regulatory sequences are contained in promoters such as cos-, tac-, trp-, tet-, trp-tet-, Ipp-, lac-, lpp-lac-, lacl^{q-}, T7-, T5-, T3-, gal-, trc-, ara-, rhaP (rhaP_{BAD})SP6-, lambda-P_{R}- or in the lambda-P_{L}-promoter, which advantageously find application in gram-negative bacteria. Further advantageous regulatory sequences are contained for example in the gram-positive promoters amy and SPO2, in the yeast or fungal promoters ADC1, MFalpha, AC, P-60, CYC1, GAPDH, TEF, rp28, ADH. Artificial promoters can also be used for regulation.

For expression in a host organism, the nucleic acid construct is advantageously inserted into a vector, for example a plasmid or a phage, particularly preferred a viral, more particular a baculoviral vector, which makes optimal expression of the genes in the host possible. Apart from plasmids and phage, vectors are also to be understood as all other vectors known by a person skilled in the art, e.g. viruses, such as SV40, CMV, baculovirus and adenovirus, transposons, IS elements, phasmids, cosmids, and linear or circular DNA. These vectors can be replicated autonomously in the host organism or can be replicated chromosomally. These vectors represent a further embodiment of the invention.

Suitable plasmids are for example in E. coli pLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-B1, λgt11 or pBdCl, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, in yeasts 2alphaM, pAG-1, YEp6, YEp13 or pEMBLYe23 or in plants pLGV23, pGHlac⁺, pBIN19, pAK2004 or pDH51. The stated plasmids represent a small selection of the possible plasmids. Further plasmids are well known by a person skilled in the art and can for example be found in the book Cloning Vectors (Eds. Pouwels P. H. et al. Elsevier, Amsterdam-New York-Oxford, 1985, ISBN 0 444 904018).

In another embodiment of the vector, the vector containing the nucleic acid construct according to the invention or the nucleic acid according to the invention can also advantageously be introduced in the form of a linear DNA into the microorganisms and integrated via heterologous or homologous recombination into the genome of the host organism. This linear DNA can consist of a linearized vector such as a plasmid or only of the nucleic acid construct or the nucleic acid according to the invention.

For optimal expression of heterologous genes in organisms, it is advantageous to alter the nucleic acid sequences corresponding to the specific “codon usage” used in the organism. The “codon usage” can easily be determined on the basis of computer evaluations of other known genes of the organism in question.

An expression cassette according to the invention is produced by fusion of a suitable promoter with a suitable coding nucleotide sequence and a terminator signal or polyadenylation signal. Common recombination and cloning techniques are used, as described for example in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

For expression in a suitable host organism, advantageously the recombinant nucleic acid construct or gene construct is inserted into a host-specific vector, which makes optimal expression of the genes in the host possible. Vectors are well known by a person skilled in the art and are given for example in “Cloning vectors” (Pouwels P. H. et al., Ed., Elsevier, Amsterdam-New York-Oxford, 1985).

3. Microbial Host Cells

Depending on the context, the term “microorganism” or “host” is to be understood broadly can mean the wild-type microorganism or a genetically altered, recombinant microorganism or both, and extends to prokaryotic or eukaryotic microorganisms as well as cell lines of higher eukaryotic organisms, in particular insect cell lines, which may be applied for generating suitable expression vectors or which may be applied for generating target polypeptides of the invention.

Using the vectors according to the invention, recombinant microorganisms can be produced, which are for example transformed with at least one vector according to the invention and can be used for producing the polypeptides according to the invention. Advantageously, the recombinant constructs according to the invention, described above, are introduced into a suitable host system and expressed. Preferably common cloning and transfection methods, known by a person skilled in the art, are used, for example coprecipitation, protoplast fusion, electroporation, retroviral transfection and the like, for expressing the stated nucleic acids in the respective expression system. Suitable systems are described for example in Current Protocols in Molecular Biology, F. Ausubel et al., Ed., Wiley Interscience, New York 1997, or Sambrook et al. Molecular Cloning: A Laboratory Manual. 2nd edition, Cold Spring Harbor Laboratory, Cold Spring Harbor Laboratory Press, Cold Spring Harbor, N.Y., 1989.

In principle, all prokaryotic or eukaryotic organisms may be considered as recombinant host organisms for the nucleic acid according to the invention or the nucleic acid construct. Microorganisms such as bacteria, fungi or yeasts are used as host organisms. Bacteria may be gram-positive or gram-negative bacteria, like bacteria of the families Enterobacteriaceae, Pseudomonadaceae, Rhizobiaceae, Streptomycetaceae or Nocardiaceae, especially preferably bacteria of the genera Escherichia, Pseudomonas, Streptomyces, Nocardia, Burkholderia, Salmonella, Agrobacterium, Clostridium or Rhodococcus. The genus and species Escherichia coli is quite especially preferred.

The host organisms according to the invention preferably contain at least one of the nucleic acid sequences, nucleic acid constructs or vectors described in the present invention.

Depending on the host organism, the organisms used in the method according to the invention are grown or cultured in a manner known by a person skilled in the art. Microorganisms are as a rule grown in a liquid medium, which contains a carbon source generally in the form of sugars, a nitrogen source generally in the form of organic nitrogen sources such as yeast extract or salts such as ammonium sulfate, trace elements such as iron, manganese and magnesium salts and optionally vitamins, at temperatures between 0° C. and 100° C., preferably between 10° C. to 60° C. with oxygen aeration. The pH of the liquid nutrient can be kept at a fixed value, i.e. regulated or not during culture. Culture can be batchwise, semi-batchwise or continuous. Nutrients can be present at the beginning of fermentation or can be supplied later, semicontinuously or continuously.

4. Recombinant Production of Target Polypeptides

The invention further relates to methods for recombinant production of target polypeptides as defined herein, wherein a polypeptide-producing microorganism is cultured, optionally the expression of the polypeptides is induced and these are isolated from the culture.

A summary of known cultivation methods can be found in the textbook by Chmiel (Bioprozesstechnik 1. Einführung in die Bioverfahrenstechnik [Bioprocess technology 1. Introduction to bioprocess technology] (Gustav Fischer Verlag, Stuttgart, 1991)) or in the textbook by Storhas (Bioreaktoren and periphere Einrichtungen [Bioreactors and peripheral equipment] (Vieweg Verlag, Braunschweig/Wiesbaden, 1994)).

The culture medium to be used must suitably meet the requirements of the respective strains. Descriptions of culture media for various microorganisms are given in the manual “Manual of Methods for General Bacteriology” of the American Society for Bacteriology (Washington D.C., USA, 1981).

These media usable according to the invention usually comprise one or more carbon sources, nitrogen sources, inorganic salts, vitamins and/or trace elements.

Preferred carbon sources are sugars, such as mono-, di- or polysaccharides. Very good carbon sources are for example glucose, fructose, mannose, galactose, ribose, sorbose, ribulose, lactose, maltose, sucrose, raffinose, starch or cellulose. Sugars can also be added to the media via complex compounds, such as molasses, or other by-products of sugar refining. It can also be advantageous to add mixtures of different carbon sources. Other possible carbon sources are oils and fats, for example soybean oil, sunflower oil, peanut oil and coconut oil, fatty acids, for example palmitic acid, stearic acid or linoleic acid, alcohols, for example glycerol, methanol or ethanol and organic acids, for example acetic acid or lactic acid.

Nitrogen sources are usually organic or inorganic nitrogen compounds or materials that contain these compounds. Examples of nitrogen sources comprise ammonia gas or ammonium salts, such as ammonium sulfate, ammonium chloride, ammonium phosphate, ammonium carbonate or ammonium nitrate, nitrates, urea, amino acids or complex nitrogen sources, such as corn-steep liquor, soya flour, soya protein, yeast extract, meat extract and others. The nitrogen sources can be used alone or as a mixture.

Inorganic salt compounds that can be present in the media comprise the chloride, phosphorus or sulfate salts of calcium, magnesium, sodium, cobalt, molybdenum, potassium, manganese, zinc, copper and iron.

Inorganic sulfur-containing compounds, for example sulfates, sulfites, dithionites, tetrathionates, thiosulfates, sulfides, as well as organic sulfur compounds, such as mercaptans and thiols, can be used as the sulfur source.

Phosphoric acid, potassium dihydrogen phosphate or dipotassium hydrogen phosphate or the corresponding sodium-containing salts can be used as the phosphorus source.

Chelating agents can be added to the medium, in order to keep the metal ions in solution. Especially suitable chelating agents comprise dihydroxyphenols, such as catechol or protocatechuate, or organic acids, such as citric acid.

The fermentation media used according to the invention usually also contain other growth factors, such as vitamins or growth promoters, which include for example biotin, riboflavin, thiamine, folic acid, nicotinic acid, pantothenate and pyridoxine. Growth factors and salts often originate from the components of complex media, such as yeast extract, molasses, corn-steep liquor and the like. Moreover, suitable precursors can be added to the culture medium. The exact composition of the compounds in the medium is strongly dependent on the respective experiment and is decided for each specific case individually. Information on media optimization can be found in the textbook “Applied Microbiol. Physiology, A Practical Approach” (Ed. P. M. Rhodes, P. F. Stanbury, IRL Press (1997) p. 53-73, ISBN 0 19 963577 3). Growth media can also be obtained from commercial suppliers, such as Standard 1 (Merck) or BHI (brain heart infusion, DIFCO) and the like.

All components of the medium are sterilized, either by heat (20 min at 1.5 bar and 121° C.) or by sterile filtration. The components can either be sterilized together, or separately if necessary. All components of the medium can be present at the start of culture or can be added either continuously or batchwise.

The culture temperature is normally between 15° C. and 45° C., preferably 25° C. to 40° C. and can be varied or kept constant during the experiment. The pH of the medium should be in the range from 5 to 8.5, preferably around 7.0. The pH for growing can be controlled during growing by adding basic compounds such as sodium hydroxide, potassium hydroxide, ammonia or ammonia water or acid compounds such as phosphoric acid or sulfuric acid. Antifoaming agents, for example fatty acid polyglycol esters, can be used for controlling foaming. To maintain the stability of plasmids, suitable selective substances, for example antibiotics, can be added to the medium. To maintain aerobic conditions, oxygen or oxygen-containing gas mixtures, for example ambient air, are fed into the culture. The temperature of the culture is normally in the range from 20° C. to 45° C. The culture is continued until a maximum of the desired product has formed. This target is normally reached within 10 hours to 160 hours.

The fermentation broth is then processed further. Depending on requirements, the biomass can be removed from the fermentation broth completely or partially by separation techniques, for example centrifugation, filtration, decanting or a combination of these methods or can be left in it completely.

If the polypeptides are not secreted in the culture medium, the cells can also be lysed and the product can be obtained from the lysate by known methods for isolation of proteins. The cells can optionally be disrupted with high-frequency ultrasound, high pressure, for example in a

French press, by osmolysis, by the action of detergents, lytic enzymes or organic solvents, by means of homogenizers or by a combination of several of the aforementioned methods.

The expressed polypeptides can be purified by known chromatographic techniques, such as molecular sieve chromatography (gel filtration), such as Q-sepharose chromatography, ion exchange chromatography and hydrophobic chromatography, and with other usual techniques such as ultrafiltration, crystallization, salting-out, dialysis and native gel electrophoresis. Suitable methods are described for example in Cooper, T. G., Biochemische Arbeitsmethoden [Biochemical processes], Verlag Walter de Gruyter, Berlin, N.Y. or in Scopes, R., Protein Purification, Springer Verlag, New York, Heidelberg, Berlin.

For isolating the recombinant protein, it can be advantageous to use vector systems or oligonucleotides, which lengthen the cDNA by defined nucleotide sequences and therefore code for altered polypeptides or fusion proteins, which for example serve for easier purification. Suitable modifications of this type are for example so-called “tags” functioning as anchors, for example the modification known as hexa-histidine anchor or epitopes that can be recognized as antigens of antibodies (described for example in Harlow, E. and Lane, D., 1988, Antibodies: A Laboratory Manual. Cold Spring Harbor (N.Y.) Press). These anchors can serve for attaching the proteins to a solid carrier, for example a polymer matrix, which can for example be used as packing in a chromatography column, or can be used on a microtiter plate or on some other carrier.

At the same time these anchors can also be used for recognition of the proteins. For recognition of the proteins, it is moreover also possible to use usual markers, such as fluorescent dyes, enzyme markers, which form a detectable reaction product after reaction with a substrate, or radioactive markers, alone or in combination with the anchors for derivatization of the proteins.

5. ncAAs

The term “ncAA” refers generally to any non-canonical or non-natural amino acid or amino acid residue which is not among the 22 naturally occurring proteinogenic amino acids. The term encompasses also the corresponding salt forms of the such ncAAs.

While the present invention is illustrated in more detail with Expression systems applying the ncAA pyrrolysine (pyl), the invention is not limited to said particular ncAA. Particular preferred ncAAs are those which may be post-transitionally further modified.

Non-limiting examples of other post-translationally modifiable residues are:

| | |
|---|---|
| p-acetylphenylalanine, | Reaction with hydrazides |
| m-acetylphenylalanine | and hydroxylamine |
| p-(3-oxobutanoy)phenylalanine | |
| p-isopropylthiocarbonyl-phenylalanine | Reaction with amines |
| p-ethylthiocarbonyl-phenylalanine | |
| p-azidophenylalanine | Cycloaddition reactions |
| p-propargyloxyphenylalanine | |
| Phenylselenidylalanine | Thiol reactive |
| p-benzoyl-l-phenylalanine | UV reactive |
| p-boronophenylalanine | Glucose binding |

A particular class of ncAAs are also those as described in WO2012/104422 or WO2015/107064. Therein ncAAs, in particular lysine-based ncAAs, are described comprising cyclooctynyl or transcyclooctynyl analog groups suitable for particularly favorable posttranslational modification reactions, also known as copper-free click reactions, as further described in WO2012/104422 or WO2015/107064.

With respect to the different forms of click chemistry reference may be made to Blackman et al., J. Am. Chem. Soc. 2008, 130, 13518-13519; Kolb et al., Angew Chem Int Ed Engl 2001, 40:2004; Devaraj et al., Angew Chem Int Ed Engl 2009, 48:7013; Devaraj et al., Bioconjugate Chem 2008, 19:2297; Devaraj et al., Angew Chem Int Ed Engl 2010, 49:2869; WO 2010/119389 A2; WO 2010/051530 A2; Agard et al., J Am Chem Soc 2004, 126:15046; WO 2006/050262 A2; Chang et al., Proc Natl Acad Sci USA 2010, 107:1821; Neef and Schultz, Angew Chem Int Ed Engl 2009, 48:1498.

6. Preparation of Engineered Target Polypeptides (TP)

The present invention also relates to a process for preparing a target polypeptide (TP) having one or more ncAA groups, the process comprising:

a) providing a translation system comprising:
(i) an aminoacyl tRNA synthetase, or a polynucleotide encoding it;
(ii) a ncAA or salt thereof;
(iii) a tRNA having an anticodon to a selector codon, or a polynucleotide encoding said tRNA; and
(iv) a polynucleotide encoding the TP and comprising one or more than one selector codon(s),

wherein the aminoacyl tRNA synthetase (i) is capable of specifically acylating the tRNA (iii) with the compound or salt (ii);

b) allowing translation of the polynucleotide (iv); and

c) optionally recovering the resulting polypeptide.

With respect to meanings and preferred embodiments of “aminoacyl tRNA synthetases”, and “tRNAs” and “ncAAs” as applied in this method, reference is also made to the corresponding sections herein above.

The term “translation system” generally has the meaning as defined above. The translation system may be an in vivo or an in vitro translation system.

An “in vitro translation” system may be a cell-free translation system. A “cell-free” translation system is a system for synthesizing a desired protein by obtaining protein factors required for mRNA translation, e.g., in form of a cell extract, followed by reconstituting this reaction in vitro. Such cell-free systems and their use for protein synthesis are known in the art. Examples include extracts of E. coli, wheat germ extract, or rabbit reticulocyte lysate (Spirin and Swartz, Cell-free Protein Synthesis, Wiley VCH Verlag, Weinheim, Germany, 2008).

Preferably, the translation system used in the process of the invention is an “in vivo translation system”. An in vivo translation system can be a cell, e.g. a prokaryotic or eukaryotic cell. The cell can be a bacterial cell, e.g. E. coli; a fungal cell such as a yeast cell, e.g. S. cerevisiae; a plant cell, or an animal cell such as an insect cell, such as Sf21, or a mammalian cell, e.g. a HeLa cell. Eukaryotic cells used for polypeptide expression may be single cells or parts of a multicellular organism, preferably single cells.

According to a particular embodiment, the translation system is an insect cell, in particular of the genus Spodoptera, preferably Spodoptera frugiperda, most preferably Sf21 (DSMZ Nr. ACC119).

A translation system useful for preparation of TPs of the invention comprises, in particular, an aminoacyl tRNA synthetase, or a polynucleotide encoding it; a ncAA or salt thereof; a tRNA having an anticodon to a selector codon, or a polynucleotide encoding said tRNA; a polynucleotide encoding the TP of the invention and comprising one or more than one selector codon(s) in its coding sequence.

For example, polynucleotides encoding the aminoacyl tRNA synthetase, the tRNA and the polypeptide of the invention may be introduced into a cell by transfection/transformation methods known in the art.

The processes of the invention utilize an aminoacyl tRNA synthetase/tRNA (RS/tRNA) pair. Preferably, the RS/tRNA pair used in the processes of the invention is “orthogonal” to the translation system.

The tRNA and the RS used in the processes of the invention can be naturally occurring or can be derived by mutation of a naturally occurring tRNA and/or RS from a variety of organisms. In various embodiments, the tRNA and RS are derived from at least one organism. In another embodiment, the tRNA is derived from a naturally occurring or from a mutated naturally occurring tRNA of a first organism and the RS is derived from naturally occurring or from a mutated naturally occurring RS of a second organism.

A suitable tRNA/RS pair may be selected from libraries of mutant tRNA and RS, e.g. based on the results of a library screening. Alternatively, a suitable tRNA/RS pair may be a heterologous tRNA/synthetase pair that is imported from a source species into the translation system. Preferably, the cell used as translation system is different from said source species.

Methods for evolving tRNA/RS pairs are described, e.g., in WO 02/085923 and WO 02/06075.

Preferably, the RS is a pyrrolysyl tRNA synthetase (PylRS) capable of acylating a tRNA with the ncAA pyrrolysin or related ncAAs.

The pyrrolysyl tRNA synthetase used in processes of the invention may be a wildtype or a genetically engineered PylRS. Examples for wildtype PylRS include, but are not limited to PylRS from archaebacteria and eubacteria such as Methanosarcina mazei, Methanosarcina barkeri, Methanococcoides burtonii, Methanosarcina acetivorans, Methanosarcina thermophila, and Desulfitobacterium hafniense, preferably Methanosarcina mazei.

Genetically engineered PylRS have been described, for example, by Neumann et al. (Nat Chem Biol 4:232, 2008), by Yanagisawa et al. (Chem Biol 2008, 15:1187), and in EP2192185A1).

According to a particular embodiment, the pyrrolysyl tRNA synthetase used for preparation of polypeptides of the invention is wildtype pyrrolysyl tRNA synthetase from M. mazei.

According to a particular embodiment, the pyrrolysyl tRNA synthetase comprises the amino acid sequence of wildtype M. mazei pyrrolysyl tRNA synthetase set forth in SEQ ID NO:27 or a functional analog thereof.

| SEQ ID NO: 27 | |
|---|---|
| MDKKPLNTLISATGLWMSRTGTIHKIKHHEVSRSKIYIEMACGDHLVVNNSRSSRTARAL | 60 |
| RHHKYRKTCKRCRVSDEDLNKFLTKANEDQTSVKVKVVSAPTRTKKAMPKSVARAPKPLE | 120 |
| NTEAAQAQPSGSKFSPAIPVSTQESVSVPASVSTSISSISTGATASALVKGNTNPITSMS | 180 |
| APVQASAPALTKSQTDRLEVLLNPKDEISLNSGKPFRELESELLSRRKKDLQQIYAEERE | 240 |
| NYLGKLEREITRFFVDRGFLEIKSPILIPLEYIERMGIDNDTELSKQIFRVDKNFCLRPM | 300 |
| LAPNLYNYLRKLDRALPDPIKIFEIGPCYRKESDGKEHLEEFTMLNFCQMGSGCTRENLE | 360 |
| SIITDFLNHLGIDFKIVGDSCMVYGDTLDVMHGDLELSSAVVGPIPLDREWGIDKPWIGA | 420 |
| GFGLERLLKVKHDFKNIKRAARSESYYNGISTNL | 454 |

According to another particular embodiment, the pyrrolysyl tRNA synthetase is pyrrolysyl tRNA synthetase from M. mazei comprising one or more than one amino acid alteration, preferably selected from amino acid substitutions Y306A and Y384F.

According to a particular embodiment, the pyrrolysyl tRNA synthetase comprises the amino acid sequence of mutant M. mazei pyrrolysyl tRNA synthetase set forth in SEQ ID NO:30 or a functional fragment thereof.

| SEQ ID NO: 30 | |
|---|---|
| MDKKPLNTLISATGLWMSRTGTIHKIKHHEVSRSKIYIEMACGDHLVVNNSRSSRTARAL | 60 |
| RHHKYRKTCKRCRVSDEDLNKFLTKANEDQTSVKVKVVSAPTRTKKAMPKSVARAPKPLE | 120 |
| NTEAAQAQPSGSKFSPAIPVSTQESVSVPASVSTSISSISTGATASALVKGNTNPITSMS | 180 |
| APVQASAPALTKSQTDRLEVLLNPKDEISLNSGKPFRELESELLSRRKKDLQQIYAEERE | 240 |
| NYLGKLEREITRFFVDRGFLEIKSPILIPLEYIERMGIDNDTELSKQIFRVDKNFCLRPM | 300 |
| LAPNLANYLRKLDRALPDPIKIFEIGPCYRKESDGKEHLEEFTMLNFCQMGSGCTRENLE | 360 |
| SIITDFLNHLGIDFKIVGDSCMVFGDTLDVMHGDLELSSAVVGPIPLDREWGIDKPWIGA | 420 |
| GFGLERLLKVKHDFKNIKRAARSESYYNGISTNL | 454 |

Any aminoacyl tRNA synthetase described herein may be used for acylation of a tRNA with the ncAA.

According to a preferred embodiment, wildtype M. mazei pyrrolysyl tRNA synthetase is used for acylation of a tRNA with a compound as those described in WO2012/104422 or WO2015/107064, in particular of formula

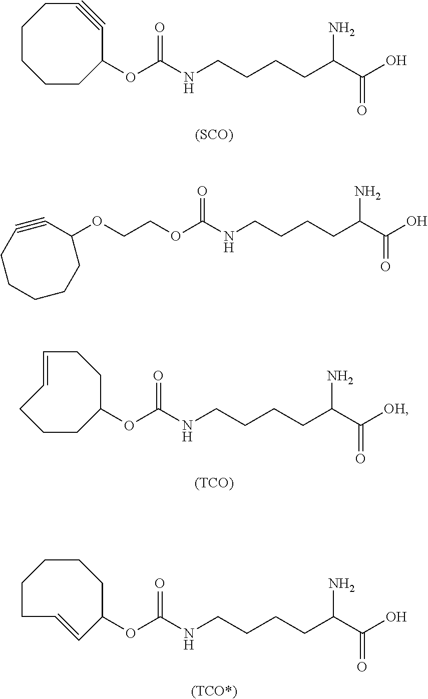

or with a compound of the formula
or a salt thereof.

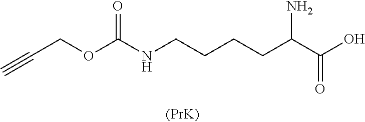

According to another preferred embodiment, a mutant M. mazei pyrrolysyl tRNA synthetase comprising amino acid substitutions Y306A and Y384F is used for acylation of a tRNA with a compound as those described in WO2012/104422 or WO2015/107064, in particular of formula

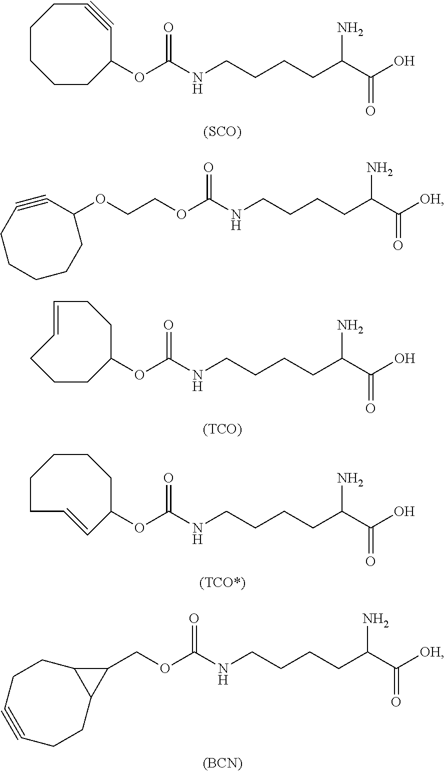

or with a compound of the formula

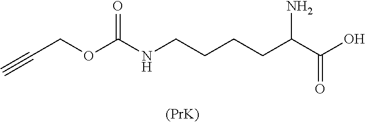

or a salt thereof.

The tRNA which is used in combination with the PylRS (tRNA^{Pyl}) may be a wildtype or a genetically engineered tRNA. Examples for wildtype tRNA^{Pyl} include, but are not limited to, tRNAs from archaebacteria and eubacteria, such as mentioned above, which facilitate translational incorporation of pyrrolysyl residues.

Selector codons utilized in processes of the present invention expand the genetic codon framework of the protein biosynthetic machinery of the translation system used. For example, a selector codon includes, e.g., a unique three base codon, a nonsense codon, such as a stop codon, e.g., an amber codon, or an opal codon, an unnatural codon, at least a four base codon or the like. A number of selector codons can be introduced into a polynucleotide encoding a TP, e.g., one or more, two or more, more than three, etc.

In one embodiment, the methods involve the use of a selector codon that is a stop codon for the incorporation of a compound of the invention. For example, an O-tRNA is generated that recognizes the stop codon, preferably the amber stop codon, and is acylated by an O-RS with a ncAA. This O-tRNA is not recognized by the naturally occurring aminoacyl-tRNA synthetases.

Conventional site-directed mutagenesis can be used to introduce the stop codon, e.g., the amber stop codon, at the site of interest into the polynucleotide sequence encoding the TP. When the O-RS, O-tRNA and the mutant gene are combined in a translation system, the unnatural amino acid is incorporated in response to the amber stop codon to give a polypeptide containing the unnatural amino acid analog, i.e. the compound of the invention, at the specified position(s).

According to particular embodiment, the tRNA^{Pyl} used in processes of the invention comprises the CUA anticodon to the amber stop codon.

Other selector codons useful for encoding compounds of the invention are rare codons. For example, when the arginine concentration in an in vitro protein synthesis reaction is reduced, the rare arginine codon, AGG, has proven to be efficient for insertion of Ala by a synthetic tRNA acylated with alanine. In this case, the synthetic tRNA competes with the naturally occurring tRNA^{Arg}, which exists as a minor species in E. coli. Some organisms do not use all triplet codons. For example, an unassigned codon AGA in Micrococcus luteus has been utilized for insertion of amino acids in an in vitro transcription/translation extract. Accordingly, any triplet codon not used by the translation system applied in the processes of the invention can serve as selector codon.

The translation system is kept for a suitable time at conditions which allow formation of the polypeptide of the invention by a ribosome. mRNA that encodes the TP and comprises one or more than one selector codon is bound by the ribosome. Then, the TP is formed by stepwise attachment of amino acids at positions encoded by codons which are bound the respective aminoacyl tRNAs. Thus, the ncAA is incorporated in the TP at the position(s) encoded by the selector codon(s).

Translation of the TP by a translation system may be effected by procedures well known in the art. To facilitate efficient translation, the components of the translation system may be mixed. Cells used as translation system are expediently cultured and kept in a suitable expression medium under conditions and for a time suitable to produce the TP. It may be required to induce expression by addition of a compound, such as arabinose, isopropyl β-D-thiogalactoside (IPTG) or tetracycline that allows transcription of the TP gene.

Optionally, after translation the TP may be recovered from the translation system. For this purpose, the TPs can be recovered and purified, either partially or substantially to homogeneity, according to procedures known to and used by those of skill in the art. Standard procedures well known in the art include, e.g., ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in making correctly folded mature proteins. High performance liquid chromatography (HPLC), affinity chromatography or other suitable methods can be employed in final purification steps where high purity is desired. Antibodies made against the unnatural amino acid or the polypeptides of the invention can be used as purification reagents, i.e. for affinity-based purification of the polypeptides.

A variety of purification/protein folding methods are well known in the art, including, e.g., those set forth in Scopes, Protein Purification, Springer, Berlin (1993); and Deutscher, Methods in Enzymology Vol. 182: Guide to Protein Purification, Academic Press (1990); and the references cited therein.

The invention is now explained in more detail by making reference to the particular, non-limiting embodiments or the subsequent experiment section.

Experimental Part

A. Materials and General Methods:

Unless stated otherwise, the cloning and expression of recombinant proteins is carried out by standard methods, as described for example in Sambrook, J., Fritsch, E. F. and Maniatis, T., Molecular Cloning: A Laboratory Manual, 2nd edition, Cold Spring Harbor Laboratory, Cold Spring Harbor Laboratory Press, Cold Spring Harbor, N.Y., 1989.

1. Plasmids and Cell Lines

1.1 Plasmids

Plasmid maps are shown in FIG. 11A to L.

pIEX-ccdb: source: Protein Expression and Purification core facility EMBL, Heidelberg

pIDK: source: Imre Berger, EMBL Grenoble

pIZT-V5/His: source: Thermo scientific, pIZTN5-His Vector Kit

pUCDM: source: Imre Berger, EMBL Grenoble

pACEBac-Dual: source: Protein Expression and Purification core facility EMBL, Heidelberg

pFastBac-Dual: Imre Berger, EMBL Grenoble

pBAD-Intein-CBD-12His: source: Plass T., Milles S., Koehler C., Schultz C., Lemke E A. Genetically encoded copper-free click chemistry. Angew Chem Int Ed Engl. 50, 3878-81 (2011)

pEvol PylRS WT: source: Plass T., Milles S., Koehler C., Schultz C., Lemke E A. Genetically encoded copper-free click chemistry. Angew Chem Int Ed Engl. 50, 3878-81 (2011)

pEvol PylRS AF: source: Plass T., Milles S., Koehler C., Schultz C., Lemke E A. Genetically encoded copper-free click chemistry. Angew Chem Int Ed Engl. 50, 3878-81 (2011)

pBAD-GFP (Y39TAG-6His): source: Plass T., Milles S., Koehler C., Schultz C., Lemke E A. Genetically encoded copper-free click chemistry. Angew Chem Int Ed Engl. 50, 3878-81 (2011)

1.2 Cell Lines

1.2.1 Insect Cells:

| | |
|---|---|
| Sf21: | Source: DSMZ ACC119 |
| | Species: insect - fall armyworm |
| | (Spodoptera frugiperda) |
| | (Spodoptera frugiperda) |
| | Cell type: ovary cells |
| Schneider 2R+: | Source: Drosophila Genomics |
| | Resource Center (DGRC) |
| | Stock number: 150 |
| DH10BacTAG WT | invention |
| DH10Bac TAG AF | invention |

1.2.2 Bacterial Cells

| | |
|---|---|
| E. coli | Source: Thermo scienitifc |
| BL21(DE3)Al | Genotype: F⁻ompT hsdS_{B} (r_{B}⁻ m_{B}⁻) |
| | gal dcm araB::T7RNAP-tetA |
| E. coli | Source: Imre Berger, EMBL Grenoble |
| DH10MultiBac^{Cre} | |
| BW23474 | Source: Imre Berger, EMBL Grenoble, |
| | CGSC (The Coli Genetic Stock Center)#: 7838 |
| | Genotype: Δ(argF-lac)169, ΔuidA4::pir-116, |
| | recA1, rpoS396(Am), endA9(del-ins)::FRT, rph-1, |
| | hsdR514, rob-1, creC510 |

2. Cell Culture

2.1 Spodoptera frugiperda cell line Sf21

Following standard Baculovirus protocols (Nie, Y., Bieniossek, C. & Berger, I. ACEMBL Expression System, User Manual. Vers. 09.11 (2009)) Sf21 cells (DSMZ ACC119) were cultured in Erlenmeyer flask at 27° C. shaking at 180 rpm, using Sf-900™ III SFM medium. Cells were split every day to 0.6*10⁶ cells/ml or every third day to 0.3*10⁶ cells/ml.

For Bacmid transfection, 3 ml per well of 0.3*10⁶ cells/ml were seeded in a 6-well multidish (Nunclon Delta Surface, Thermo scientific). Bacmid-DNA was prepared and Sf21 cell transfected using FuGENE HD Transfection Reagent (Promega). V₀-Virus was harvested after 70 hours post transfection and the V,-generation started. For small scale test expression, 100 ml of Sf21 cells at 0.6*10⁶ cells/ml were transfected with 1 ml of V₁-Virus and 1 mM of the respective ncAA was added. As negative control, a 100 ml culture was set up the same way, but without ncAA. After cell proliferation stopped, the cultures were kept another 48-60 hours at 27° C. shaking at 180 rpm. The cells were harvested at 500 rpm for 10 minutes and the pellets were stored at −20° C.

For transient transfections, Sf21 cells were seeded 15 minutes before transfection, in a 6-well multidish at a density of 0.3*10⁶ cells/ml. 3 μg of total DNA with 200 μl of medium were mixed with FuGENE HD Transfection Reagent. After an incubation time of 15 minutes, the DNA mixture was given into to the well in a drop wise manner. If required, the ncAA was added to a final concentration of 1 mM per well. For a co-transfection of two plasmids, a 1:1 ratio was used.

2.2 Drosophila Melanogaster Cell Line Schneider 2 R+ Cells

Schneider Drosophila medium (Thermo scientific) with 10% Fetal Bovine Serum (FBS) (PAA Laboratories), 2 mM Glutamine and 1% Penicillin Streptomycin was used for culturing Schneider 2 R+ cells (Drosophila Genomics Resource Center (DGRC)) (DeRenzi lab, EMBL Heidelberg) in cell culture dishes (10 mm), kept on a density of 1 Mio cells/ml. For transfection cells were seeded at a density of 0.5*10⁶ cells/ml prior to the transfection. Utilizing a 6-well multidish, 2 μg of DNA were mixed with 200 μl of medium and 12 μl of FuGENE HD Transfection Reagent, incubated for 15-20 minutes and added drop wise to each well. The ratio for co-transfections of two plasmids was always 1:1. After two days, the transfection was analyzed by microscope or flow cytometry.

1. Analyzing Sf21 Genome:

The genomic DNA of Spodoptera frugiperda was extracted using standard protocols and the genome was sequenced by the genome core facility at EMBL, Heidelberg.

Eight U6 snRNA gene could be found (U6-1-U6-8) (see Supplementary FIG. 1), using Bombyx mori snRNA U6 isoform E gene as query sequence (GenBank: AY649381.1), with at least 400 bp upstream (promoter region) and 100 bp downstream sequences (termination signal) (Supplementary FIG. 2). We decided to work with U6 promoter and the 3′termination signal out of the second scaffold (17011_2962_3036_+), which was found, and called this U6 promoter, U6(Sf21)-2.

4. Flow Cytometry Analyses

Flow cytometry analyses were done on a BD LSRFORTESSA (BD Biosciences). Therefore Sf21 or S2 cells were co-transfected with pIZT-PylRS-mCherry-GFP(Y39TAG)-6His (see FIG. 5B) and a plasmid for the tRNA expression in a 6-well multidish. After two days of incubation time, the cells were harvested at 500 rpm for 10 minutes at 4° C. and resuspended in 500 μl sterile 1× PBS. The suspension was filtered through a cell strainer (Falcon, 70 μm, Fisher scientific) and kept on ice until measurements. Data of 5 Millions cells for each sample was acquired and analyzed with FlowJo X software (FlowJo Enterprise).

B. Plasmid Preparation for Transient Transfection

For the expression of Methanosarcina mazei tRNA^{Pyl} in transient transfections, different U6 promoters were tested. The sequences of the different tRNA^{Pyl} expression constructs is shown in FIG. 3A. (SEQ ID NOs: 15 to 18)

### EXAMPLE B.1

### U6 Homo Sapiens

The U6 (homo sapiens) promoter was cloned in front of the Mm tRNA^{Pyl} gene, followed by a short 3′termination signal into the plasmid pIEx-ccdB (SEQ ID NO:50), leading to the pIEx-U6(Human)-tRNA^{Pyl}-3′term plasmid (SEQ ID NO:19).

### EXAMPLE B.2

### U6 Drosophila Melanogaster

The plasmid pIEx-U6(Dm)-2-tRNA^{Pyl}-3′term (SEQ ID NO:20) was constructed following the cloning strategy in Bianco et al. 2012¹. Bianco, A., Townsley, F. M., Greiss, S., Lang, K. & Chin, J. W. Expanding the genetic code of Drosophila melanogaster. Nat Chem Biol 8, 748-750 (2012). A four times tRNA expression cassette with the U6(Dm)-2 promoter was cloned to achieve the plasmid pIEx-U6(Dm)-2-tRNA^{Pyl}-3′term 4×. (SEQ ID NO:21)

### EXAMPLE B.3

### U6 Bombyx Mori

The tRNA cassette composed of the U6-2 promotor of Bombyx mori, followed by the tRNA^{Pyl} gene and the 3′termination signal of the snRNA U6 gene from Bombyx mori was ordered from Genewiz Inc. This 586 bp fragment was cloned between the ClaI and NcoI sites in the plasmid pIDK (SEQ ID NO:22)resulting in pIDK-U6(Bm)-2-tRNA^{Pyl}-3′term (SEQ ID NO:23)

### EXAMPLE B.4

### U6 Spodoptera Frugiperda

a) Preparation of Plasmid pIEx-U6(Sf21)-2-tRNA^{Pyl}-3′Term

To test the amber suppression in the transient system using the U6 promoter from Spodoptera frugiperda, the U6(Sf21)-2 promoter sequence (400 bp upstream the snRNA U6 gen) was followed by the Methanosarcina mazei tRNA^{Pyl} gene, ending with the corresponding 3′termination signal of the snRNA U6 gene (SEQ ID NO:2). The Methanosarcina mazei tRNA construct was taken out of an ordered gene (Genewiz Inc.) by PCR, digested with EcoRI and NotI restriction enzymes and ligated into a pIEx-ccdb plasmid (SEQ ID NO:50) (Protein Expression and Purification core facility EMBL, Heidelberg), which consists of an enhancer in front of a promoter, called IE1, and a ccdB gene, which helps to keep the background low during cloning. This plasmid was cut beforehand with the same enzymes, resulting into the plasmid pIEx-U6(Sf21)-2-tRNA^{Pyl}-3′term (FIG. 5A). (SEQ ID NO:24) To compare the different U6(Sf21) promoters (U6(SF21)1-8) the same cloning strategy was used as for the U6(Sf21)-2 tRNA construct.

b) Preparation of Plasmid pIZT-PylRS-mCherry-GFP(Y39TAG)-6His

For generating the plasmid pIZT-PylRS-mCherry-GFP(Y39TAG)-6His (FIG. 5B) (SEQ ID NO:25), first a mCherry-GFP(Y39TAG)-6His construct was cloned into pIZT-V5/His plasmid (Thermo scientific, pIZT/V5-His Vector Kit) under OpIE1 promoter using NcoI and SalI as restriction enzymes after changing the resistance gene to Ampicillin, and in a second step the Methanosarcina mazei PylRS WT gene (SEQ ID NO:26) was taken out of pEvol-PylRS by PCR and conventional restriction enzyme cloning, using KpnI and XbaI, to get the gene expressed under the OpIE2 promoter. pEvol PylRS is a plasmid, which is used for amber suppression in E. coli cells and contains in addition to the tRNA^{Pyl} gene under a constitutive promoter two Methanosarcina mazei PylRS genes, one under a constitutive promoter (gInS) and one under an arabinose inducible promoter. pIZT-PylRS-mCherry-GFP(WT) (SEQ ID NO:28) was constructed the same way.

C. Plasmid Preparation for E. Coli Transformation

The plasmid coding for the reporter construct GFP(Y39TAG)-6His was cloned as previously described (Plass T., Milles S., Koehler C., Schultz C., Lemke E A. Genetically encoded copper-free click chemistry. Angew Chem Int Ed Engl. 50, 3878-81 (2011)), as well as pEvol PylRS WT and pEvol PylRS AF. The plasmid pEvol PylRS AF contains the same elements as the plasmid pEvol PylRS WT, but the PylRS genes includes two point mutations (Y346A and Y384F). (SEQ ID NO:29)

The plasmid pBAD-GFP(Y39TAG)-6His (SEQ ID NO:31) contains a GFP gene with an amber mutation at position Y39 and a C-terminal 6His-tag, under an arabinose inducible promoter.

D. Preparation of DH10Bac TAG Cell Line

### EXAMPLE D.1

### Construction of Amber Suppression Bacmid (DH10BacTAG)

For generating a bacmid, which contains PylRS synthetase and tRNA^{Pyl} for amber suppression, we first cloned U6(Sf21)-2-tRNA^{Pyl}-3′term (SEQ ID NO:2) into the pUCDM (Imre Berger, EMBL Grenoble) (SEQ ID NO:32) plasmid using ClaI and XbaI as restriction enzymes, followed by adding the MM PylRS or MM PylRS AF under the p10 promoter, cutting with NsiI and XhoI. The pUCDM is used as a transfer plasmid in modifying a Bacmid backbone and it contains two insect promoters for parallel expression of two genes. For all cloning steps of the pUCDM plasmid, BW23474 cells were used for propagation. The resulting vector pUCDM-PylRSWT-U6(Sf21)-2-tRNA^{Pyl}-3′term (FIG. 5C) (SEQ ID NO:33) (U6-tRNA cassette 5′→3′ and PylRS gene 3′→5′) was transformed into electro-competent E. coli DH10MultiBac^{Cre} cells (Imre Berger, EMBL Grenoble), following the protocol of Berger, I., Fitzgerald, D. J. & Richmond, T. J. Baculovirus expression system for heterologous multiprotein complexes. Nat Biotechnol 22, 1583-1587 (2004); and Fitzgerald, D. J. et al. Protein complex expression by using multigene baculoviral vectors. Nat Methods 3, 1021-1032 (2006).

Eight blue colonies were picked, Bacmid-DNA was prepared and Sf21 cells were transfected as described in Craig, A. & Berger, I. ACEMBL Expression System Series, MultiBacTurbo, Multi-Protein Expression in Insect Cells, User Manual. Vers. 3.0 (2011). From each of the eight picked colonies a glycerol stock was prepared in parallel. V₀-virus was harvested after 60 hours of incubation and the V₁-generation was started. After cell proliferation stopped, the cells were harvested after additional 60 hours. The cell pellet was taken up in 4× PBS (phosphate-buffered saline) (pH8), resulting in 1 Mio. cells/ml. 10 μl of sample was mixed with same amount of SDS loading dye, incubated at 95° C. for 10 minutes and loaded on SDS-PAGE before performing a Western Blot, using Anti-PylRS (Rat; Eurogentec) as primary antibody (FIG. 6). As control, PylRS-6His was loaded, which was beforehand expressed in E. coli and purified using standard purification protocols. From the glycerol stock, corresponding to Bacmid #2, electro-competent cells were prepared, the so called DH10BacTAG (WT). This procedure was done for both, the pUCDM-U6(Sf21)-2-tRNA^{Pyl}-3′term containing the PylRS WT as well as the PylRS AF variant vector pUCDM-PylRSAF-U6(Sf21)-2-tRNA^{Pyl}-3′term (SEQ ID NO:34) (PylRS gene 5′→3′, U6-tRNA cassette 3′→5′) (Western Blot not shown), which leads to the E. coli strain DH10BacTAG (AF).

E. Plasmid Preparation for Stabile (Bacmid) Transfection

Different Plasmids were construed for expressing amber mutated proteins

### EXAMPLE E1

### pACEBac-Dual-GFP(Y39TAG)-6His

A reporter plasmid was constructed, therefore GFP(Y39TAG)-6His was cloned into pACEBac-Dual-plasmid (SEQ ID NO:35) (Protein Expression and Purification core facility, EMBL Heidelberg) under the PH (Polyhedrin) promoter, using BamHI and PstI as restriction enzymes. The resulting pACEBac-Dual-GFP(Y39TAG)-6His (SEQ ID NO:36) (FIG. 5D) was transformed into DH10BacTAG (WT) and DH10BacTAG (AF) (see Example D1) and thereby integrated into the Tn7 side.

### EXAMPLE E2

### pACEBac-Dual-Herceptin-6His

The F_{ab} fragment gen, composed of the coding sequences of variable and constant regions of the heavy (SEQ ID NO:37) and light chain (SEQ ID NO:39) of Herceptin (Anti-Her), was ordered codon optimized for Sf21 cells and cloned with a C-terminal 6His-tag at the heavy chain in pACEBac-Dual plasmid under the p10 promoter and PH promoter, respectively. Two amber mutations at position A121 and A132 of the heavy chain were inserted individually by quick change PCR into the resulting plasmid, pACEBac-Dual-Herceptin-6His, (SEQ ID NO:41) (Herceptin light chain 5′→3′, heavy chain 3′→5′) (FIG. 5E).

### EXAMPLE E3

### pFastBac-Dual-6HisTAF11/TAF13

pFastBac-Dual-6HisTAF11/TAF13 (FIG. 5F) (SEQ ID NO:42) (TAF11 5′→3′ and TAF13 3′→5′) was generated by cloning the TAF11 (Transcription initiation factor TFIID subunit 11) gene SEQ ID NO:43 with an N-terminal 6His-tag using RsrII and EcoRI as restriction enzymes into pFastBac-Dual plasmid under the PH promoter, as well as TAF13 (Transcription initiation factor TFIID subunit 13) SEQ ID NO:45 into the resulting plasmid after the p10 promoter. Single amber stop codons were introduced into the TAF13 gene at the positions R30, E34 and R35 performing quick change PCR.

### EXAMPLE E4

### BAD-TBP-Intein-CBD-12His

Residues 155-333 of the TATA-Box binding protein (TBP) (SEQ ID NO:48) were cloned into pBAD-Intein-CBD-12His plasmid by conventional restriction side cloning using NcoI and SpeI as enzymes, resulting in pBAD-TBP-Intein-CBD-12His. (FIG. 5G) (SEQ ID NO:49)

F. Protein Expression and Purification

### EXAMPLE F.1

### Expression of GFP(Y39TAG) in E. Coli and Purification

The plasmid coding for the amber mutant (Y39TAG) of GFP (pBAD-GFP(Y39TAG-6His) was co-transformed with pEvol PylRS WT or pEvol PylRS AF, respectively, and expressed in E. coli BL21(DE3) Al cells at 37° C. in TB-FB medium. The ncAA was added, when the OD₆₀₀ of 0.2 was reached and at OD₆₀₀=0.4 the expression was induced with 0.02% Arabinose. The cells were harvested after 6-8 hours and the pellets frozen and stored at −20° C.

The cell pellet was resuspended in 10 ml 4× PBS buffer (1mM PMSF, 90.2 mM TCEP, 5 mM imidazol) per 11 expression culture and sonicated for 30 seconds. After spinning down at 15000 rpm for 1 hour at 4° C., the soluble fraction was incubated on nickel beads for at least 30 minutes. Impurities were removed by washing with 4× PBS with 10 mM imidazol and finally the protein was eluted with 500 mM imidazol in 4× PBS buffer. The proteins were loaded on a NuPAGE Gradient gel (4-20%) (Invitrogen) and run in MOPS buffer. If necessary, the protein was further purified over a gelfiltration column.

### EXAMPLE F.2

### Expression of GFP(Y39TAG) in the Baculovirus-System

The plasmid pACEBac-Dual-GFP(Y39TAG)-6His (see FIG. 5D) coding for the amber mutant (Y39TAG) of GFP was transformed into DH10BacTAG cells (WT and AF variant) as prepared according to Example E1), and plated on blue/white selection plates, containing X-Gal and IPTG, as well as Ampicillin (100 μg/ml), Kanamycin (30 μg/ml), Tetracycline (10 μg/ml) and Gentamycin (10 μg/ml). Four white colonies were picked and Bacmid-DNA prepared. After transfecting Sf21 cells, as described herein beforehand, the four V₀-Virus preparations were harvested after 60 hours. V₁-Virus was produced using all four V₀-Viruses in parallel and for each 1 ml of Virus was added to 100 ml of fresh Sf21 cells. Five cultures were set up in the same way, one for each of the four V₁-Viruses, in which ncAA at a final concentration of 1 mM was added and 1 culture without ncAA, as a negative control, for which V₁-Virus resulting from the first Bacmid-DNA was added. After cell propagation stopped, the cells were harvested after additional 48-60 hours.

The purification was the same as for GFP expressed in E. coli, with only on difference. After the sonication step, the lysate was centrifuged at 40.000 rpm at 4° C. using a Beckman ultracentrifuge (SW Ti60 rotor). The purification success after incorporation of propargyl-lysine (PrK) and SCO-L-lysine was analyzed by SDS-PAGE (FIG. 12).

Mass spectrometry analysis of GFP(Y39TAG) with propagyl-lysine (PrK) and SCO—L-lysine confirm the incorporation of these unnatural amino acids into the protein GFP (FIGS. 7 and 8) as well as Herceptin (FIG. 9).

### EXAMPLE F.3

### Expression and Purification of Herceptin F_{ab} Fragment in the Baculovirus-System

For the expression of the Herceptin F_{ab} fragment carrying an amber stop codon at position 121 or 132 in the heavy chain the plasmid pACEBac-Dual-Herceptin-6His (see FIG. 5E) was transformed in DH10BacTAG WT and AF as prepared according to Example E2), following the same protocol as for the GFP(Y39TAG) expression in the Baculovirus-system expression was performed with and without PrK and with and without BOC-lysine. Also for this purification, we followed the same protocol as for the GFP(Y39TAG)-6His protocol and analyzed the purity on a SDS-PAGE. FIG. 13A shows the expression with or without PrK; FIG. 13B shows the expression with or without BOC-lysine.

### EXAMPLE F.4

### Expression and Purification of the TAF Complex (TAF11/TAF13) in the Baculovirus-System

For expressing the TAF complex we transformed plasmid pACEBac-Dual-6HisTAF11/TAF13 (see FIG. 5F) in the DH10BacTAG AF variant as prepared according to Example E3. This was done for the TAF11/TAF13 WT complex, as well as for the amber mutants. We followed the same expression protocol as for the GFP(Y39TAG)-6His expression in the Baculovirus-system. The cell pellet was resuspended in 150 ml Tris buffer (25 mM Tris, 150 mM NaCl, 5 mM imidazol, complete protease inhibitor mix (Roche Diagnostics), Leupeptin and Pepstatin) per 1 liter expression culture. Lyse the cells by several rounds of flash freezing and thawing. Spin down the cells at 40000 rpm at 4° C. (Beckman SWTi60 rotor). Incubate supernatant on Nickel beads for 1-2 hours and elute protein after several washing steps with increasing imidazol concentrations. After buffer exchange, the protein was loaded on a Q-Sepharose column. To finalize the purification procedure, the protein was injected on a Superdex column and a analyzed by SDS-PAGE.

### EXAMPLE F.5

### E. Coli Expression and Purification of TATA-Box Binding Protein (TBP), Residues 155-333

pBAD-TBP-Intein-CBD-12His (see FIG. 5G; as prepared in Example E4) was transformed into

E. coli BL21(DE3) Al cells and expressed in TB-FP medium at 18° C. over night. Cells were harvested by centrifugation (450 0 rpm, 20 min., 4° C.) and stored at −20° C.

The cells of 1 liter expression culture were lysed in 20 ml TBP lysis buffer (25 mM Tris, 1 M NaCl, 10 mM imidazol, 1 mM PMSF, 0.2 mM TCEP, pH 8) using a sonicator. After spinning down the insoluble fraction, the cleared supernatant was loaded on a beforehand equilibrated Ni-column. Washing was done with increasing concentration of imidazol and the protein was finally eluted. To cleave of the Intein-CDB-12His tag, the protein was incubated over night at RT with 100 mM β-mercaptoethanol (BME). An afterwards dialyzing step, exchanged the buffer back to TBP lysis buffer, without imidazol, and the protein was purified further with Ni beads. The purity of the protein was checked by SDS-PAGE analysis.

### EXAMPLE F.6

### Transient Transfection of S2 Cells and Flow Cytometry Analyses

As a control measurement, we tested the Drosophila melanogaster U6-tRNA construct (U6(Dm)) in Schneider 2 cells. Therefore, we performed a transient transfection, using pIEx-U6(Dm)-2-tRNA^{Pyl}-3′term, as well as pIEx-U6(Dm)-2-tRNA^{Pyl}-3′term(4×) in a co-transfection with pIZT-PylRS-mCherry-GFP(Y39TAG). The expression yield was analyzed by Flow cytometry (see FIG. 4). The GFP signal reports a working amber suppression system, which is clearly shown comparing the signal of with and without ncAA in the green channel.

### EXAMPLE F.7

### Transient Transfection of Sf21 Cells and Flow Cytometry Analyses

Sf21 cell were transiently transfected with the reporter plasmid, which includes also the PylRS gene (pIZT-PylRS-mCherry-GFP(Y39TAG) as well as one out of four tRNA expressing plasmids. These plasmids are pIEx-U6(Human)-tRNA^{Pyl}-3′term, pIEx-U6(Dm)-tRNS^{Pyl}-3′term, pIDK-U6(Bm)-2-tRNA^{Pyl}-3′term and pIEx-U6(Sf21)-2-tRNA^{Pyl}-3′term, which all contain the gene for tRNA^{Pyl}, an U6 promoter and a 3′termination signal. We show, that amber suppression in Sf21 cells in the transient system is only working, if the tRNA expression cassette containing the U6(Sf21)-2 promoter is used and that there is no amber suppression, if one of the other promoter sequences are transfected or if there is no ncAA added. This result is obtained by analyzing the GFP signal in a flow cytometer (FIG. 3B).

G. Click Reactions

### EXAMPLE G.1

### Copper-Catalyzed Alkyne-Azide Cycloaddition (CuAAC)

Purified protein, which contains an ncAA (Propargyllysine, PrK) with an alkyne group incorporated at the amber stop codon side, was exchanged to 1× PBS buffer pH7.5 (0.2 mM TCEP) and 5 nmol were used for the click reaction, following the protocol in Tyagi, S. and Lemke, E. A. Tyagi, S. & Lemke, E. A. Genetically encoded click chemistry for single-molecule FRET of proteins. Methods Cell Biol 113, 169-187 (2013). Cycloadditon reactions were followed up by SDS-PAGE (see FIG. 13C for Herceptin F_{ab} fragment 132PrK mutant).

### EXAMPLE G.2

### Strain-Promoted Alkyne-Azide Cycloaddition (SPAAC)

Protein, expressed in the presence of 1 mM of SCO-L-Lysine (Sichem), was purified and exchanged into 1× PBS buffer (pH8). For the labeling reaction 5 nmol of protein mixed with 25 nmol of TAMRA-H-tetrazine (Jena Biosciences) were incubated over night at RT. Plass, T., Milles, S., Koehler, C., Schultz, C. & Lemke, E. A. Genetically encoded copper-free click chemistry. Angew Chem Int Ed Engl 50, 3878-3881 (2011). The labeling reaction was confirmed by fluorescent scan of an SDS-PAGE FIG. 10 shows the results of a SPACC reaction of Herceptin F_{ab} 121SCO reacted with TAMRA-H-tetrazine and Herceptin F_{ab} wildtype (FIG. 10A fluorescent scan; FIG. 10B Protein staining with Coomassie Blue). As can be seen Her121SCO is selectively marked.

The documents as cited herein are all incorporated by reference.

### APPLIED SEQUENCES

| SEQ ID NO: | Description | Type |
|---|---|---|
| 1 | U6-1(Sf21)-tRNA^{Pyl}-3term (U6-1) | NS |
| 2 | U6-2(Sf21)-tRNA^{Pyl}-3term (U6-2) | NS |
| 3 | U6-3(Sf21)-tRNA^{Pyl}-3term (U6-3) | NS |
| 4 | U6-4(Sf21)-tRNA^{Pyl}-3term (U6-4) | NS |
| 5 | U6-5(Sf21)-tRNA^{Pyl}-3term (U6-5) | NS |
| 6 | U6-6(Sf21)-tRNA^{Pyl}-3term (U6-6) | NS |
| 7 | U6-7(Sf21)-tRNA^{Pyl}-3term (U6-7) | NS |
| 8 | U6-8(Sf21)-tRNA^{Pyl}-3term (U6-8) | NS |
| 9 | tRNA^{pyl} | NS |
| 10 | snRNA U6 Homo sapiens | NS |
| 11 | snRNA U6 D. melanogaster | NS |
| 12 | snRNA U6 S. frugiperda | NS |
| 13 | snRNA U6 B. mori | NS |
| 14 | snRNA U6 B. mori | NS |
| 15 | U6(Human)-tRNA^{pyl}-3term | NS |
| 16 | U6(Dm)-tRNA^{pyl}-3term | NS |
| 17 | U6(Bm)-tRNA^{pyl}-3term | NS |
| 18 | U6(Sf)-tRNA^{pyl}-3term | NS |
| 19 | plEx-U6(human)-2- tRNA^{pyl}-3term | NS |
| 20 | plEx-U6(Dm)-2- tRNA^{pyl}-3term | NS |
| 21 | plEx-U6(Dm)-2- tRNA^{pyl}-3term 4x | NS |
| 22 | plDK | NS |
| 23 | plDK-U6(Bm)-2- tRNA^{pyl}-3term | NS |
| 24 | plEx-U6(Sf)-2- tRNA^{pyl}-3term | NS |
| 25 | plZT-PylRS-mCherry-GFP(Y39TAG) | NS |
| 26 | PylRS WT | NS |
| 27 | PylRS WT | AS |
| 28 | plZT-PylRS-mCherry-GFP(WT) | NS |
| 29 | PylRS AF | NS |
| 30 | PylRS AF | AS |
| 31 | pBAD-GFP(Y39TAG)-6His | NS |
| 32 | pUCDM | NS |
| 33 | pUCDM-PylRS WT-U6(Sf)-2- tRNA^{pyl}-3term | NS |
| 34 | pUCDM-PylRS AF-U6(Sf)-2- tRNA^{pyl}-3term | NS |
| 35 | pACEBac-DUAL | NS |
| 36 | pACEBac-DUAL-GFP(Y39TAG)-6His | NS |
| 37 | Herceptin HC-6His | NS |
| 38 | Herceptin HC-6His | AS |
| 39 | Herceptin LC | NS |
| 40 | Herceptin LC | AS |
| 41 | pACEBac-DUAL-Herceptin-6His | NS |
| 42 | pFastBac-DUAL-6HisTAF11/TAF13 | NS |
| 43 | 6His-TAF11 | NS |
| 44 | 6His-TAF11 | AS |
| 45 | TAF13 | NS |
| 46 | TAF13 | AS |
| 47 | 6His-TBP | NS |
| 48 | 6His-TBP | AS |
| 49 | pBAD-TBP-lnt-CBD-12His | NS |
| 50 | plEx-ccdB | NS |

NS: Nucleotide sequence
AS: Amino acid sequence

1. A method of preparing a target polypeptide (TP) comprising in its amino acid sequence one or more, identical or different, non-canonical amino acid (ncAA) residues, which method comprises the steps of
a) expressing said TP in an insect cell line (ICL), in the presence of said one or more ncAAs, wherein the TP encoding nucleotide sequence (CS^{TP}) comprises one or more selector codons encoding said one or more ncAA residues; and
concomitantly or sequentially in any order b) expressing in said ICL one or more orthogonal bacterial aminoacyl tRNA synthetase/tRNA^{ncAA} (O-RS/O-tRNA^{ncAA}) pairs required for introducing said one or more ncAA residues into the amino acid sequence of said TP, wherein the coding sequences for said one or more bacterial tRNA^{ncAA} (CS^{tRNAncAA}) are under the control of an insect-cell derived regulatory sequence (RS^{IC}); and
c) optionally recovering the expressed TP.

2. The method of claim 1, wherein said ICL is transfected or transduced with one or more baculoviral vectors carrying the genetic information required for expressing said TP and said one or more O-RS/O-tRNA^{ncAA} pairs.

3. The method of claim 1, wherein said RS^{IC} is selected from
a) regulatory sequences of insect snRNA U6 genes, and
b) regulatory sequences of insect tRNA genes, in particular H1 regulatory sequences.

4. The method of one of the preceding claims, wherein said RS^{IC} comprises a U6 promoter, wherein said U6 promoter is selected from nucleotide sequences corresponding to nucleotide residues
a) 1 to 400 of SEQ ID NO. 1
b) 1 to 392 of SEQ ID NO: 2
c) 1 to 385 of SEQ ID NO: 3 to 8
d) functional fragments thereof comprising a partial sequence of anyone of the nucleotide sequences as defined in a) to c)

5. The method of anyone of the preceding claims, wherein said ICL is selected from Spodoptera cell lines, in particular Spodoptera frugiperda cell lines, preferably Sf21 (DSMZ Nr. ACC119); or Drosophila cell lines, in particular Drosophila melanogaster cell lines preferably Schneider-2 R+ (Drosophila Genomics Research Center (DGRC) stock number 150).

6. A baculoviral vector comprising the coding sequences of one or more orthogonal bacterial aminoacyl tRNA synthetase/tRNA^{ncAA} (O-RS/O-tRNA^{ncAA}) pairs, wherein said bacterial tRNA^{ncAA} coding sequence (CS^{tRNAncAA}) is placed under the control of an insect-cell derived regulatory sequence (RS^{IC}).

7. The vector of claim 6, wherein said RS^{IC} comprises a U6 promoter, wherein said U6 promoter is selected from nucleotide sequences corresponding to nucleotide residues
a) 1 to 400 of SEQ ID NO. 1
b) 1 to 392 of SEQ ID NO: 2
c) 1 to 385 of SEQ ID NO: 3 to 8
d) functional fragments thereof comprising a partial sequence of anyone of the nucleotide sequences as defined in a) to c)

8. An insect cell line (ICL) capable of expressing one or more orthogonal bacterial aminoacyl tRNA synthetase/tRNA^{ncAA} O-RS/O-tRNA^{ncAA} pairs required for introducing at one or more ncAA residues into the amino acid sequence of a TP to be co-expressed by said ICL, wherein each bacterial tRNA^{ncAA} coding sequence is expressed under the control of an insect-cell derived regulatory sequence (RS^{IC}).

9. The ICL of one claim 8, wherein said ICL is transfected or transduced with one or more baculoviral vectors carrying the genetic information required for expressing said TP and said one or more O-RS/O-tRNA^{ncAA} pairs, in particular one or more vectors as defined in one of the claims 6 and 7.

10. The ICL of one of the claim 8 or 9, wherein said RS^{IC} comprises a U6 promoter, wherein said U6 promoter is selected from nucleotide sequences corresponding to nucleotide residues
a) 1 to 400 of SEQ ID NO. 1
b) 1 to 392 of SEQ ID NO: 2
c) 1 to 385 of SEQ ID NO: 3 to 8
d) functional fragments thereof comprising a partial sequence of anyone of the nucleotide sequences as defined in a) to c)

11. The ICL of anyone of the claims 8 to 10, wherein said ICL is selected from Spodoptera cell lines, in particular Spodoptera frugiperda cell lines, preferably Sf21 (DSMZ Nr. ACC119);
or Drosophila cell lines in particular Drosophila melanogaster cell lines preferably Schneider-2 R+ (DGRC 150).

12. An expression cassette encoding tRNA^{pyl} selected from U6-1 to U6-8 according to SEQ ID Nos: 1 to 8 or sequences having a degree of sequence identity of at least 40% retaining their ability to functionally express bacterial tRNA^{pyl} in insect cells.

13. A target protein (TP) comprising in its amino acid sequence one or more identical or different non-canonical amino acid (ncAA) residues, obtained by expressing the TP encoding nucleotide sequence (CS^{TP}) in an insect cell line (ICL), in the presence of said one or more ncAAs, wherein said CS^{TP} comprises one or more selector codons encoding said one or more ncAA residues;
obtained by a method of anyone of the claims 1 to 5, optionally further chemically modified by performing a click reaction with said at least one non-canonical amino acid (ncAA) residue contained in its amino acid sequence.

14. A kit for preparing a recombinant target polypeptide TP having one or more ncAA residues, comprising at least one ncAA or salt thereof and further comprising at least one baculoviral vector of one of the claim 6 or 7.

15. A promoter sequence is selected from nucleotide sequences corresponding to nucleotide residues
a) 1 to 400 of SEQ ID NO. 1
b) 1 to 392 of SEQ ID NO: 2
c) 1 to 385 of SEQ ID NO: 3 to 8
d) functional fragments thereof comprising a partial sequence of anyone of the nucleotide sequences as defined in a) to c)


SEQUENCE LISTING (50 sequences)

SEQ ID NO: 1 - DNA, 569 bp, Artificial Sequence
  tRNA(Pyl) expression cassette
  promoter (1)..(400)
  tRNA (401)..(469): tRNA sequence
  3′UTR (470)..(569): Termination signal
ataagttgagttatggcttaaaaaaaaggttattttttttctatttcatactgttaaaaatcaacgcaatttacaatctgggaaatgaaatatccaataattaagttagggttacgaagtaattggaatatcgattcaattgtaatcgatttacggtacagagttcatactatttacgaaaatgctttaagtatttctatgatgatcggatgatttatttaattaaaataataaaatctattagaattacagtattcagagttaaaactaaataattatctacataattaatataagtcgattcacatttactcatcgattattatatttttaatctgtgcaactctgacttgacattgacatgcaatcaatgacatcgatcggcaccaagtatatgtttggaaacctgatcatgtagatcgaatggactctaaatccgttcagccgggttagattcccggggtttccgttttttgtaatcgtagatacaatgtcgaagattctggcccgtttattgtacatgtgtcccgcgttgctgactccccaaattctggtgttagtttaaaacc

SEQ ID NO: 2 - DNA, 561 bp, Artificial Sequence
  tRNA(Pyl) expression cassette
  promoter (1)..(392)
  tRNA (393)..(461): tRNA sequence
  3′UTR (462)..(561): Termination signal
acttaactactcaaaaagtgagggccagcagctcgaccaatgtaaaaccttgcgaggtgcgaggttaccggggacccaatcaaagagtataataactatagggaaaggcccaaccccccccccccccactgtatgtaaaaatataagacctatttctcaacctataaacctatgcaataaaacatccactagattagtctagtgactagactagaccattgttagttaacagtagttcggctagatggcgccaaattggttcttttagtgaacggtagatggcgctgtactcaatcttcatacaaatcatgttaaatgtatgggattctacatcgcgctatcaaagttttcattgtgtttgtgaagggtacaataattttgccttggcaagtggaaacctgatcatgtagatcgaatggactctaaatccgttcagccgggttagattcccggggtttccgttttttgaagagtttcagtttggtatggtttttctattttcaaattggtatgagggagtaagcataatcaaatttaatttcttttgttaaactttagctt

SEQ ID NO: 3 - DNA, 554 bp, Artificial Sequence
  tRNA(Pyl) expression cassette
  promoter (1)..(385)
  tRNA (386)..(454): tRNA sequence
  3′UTR (455)..(554): Termination signal
agatctacgaattgttatttcgactttaatttttattaaactacgtaattattgttttatttttcaatgagtttcgtattacaaattgttctaatgtttacctacatgtttaaaagatttcggcactgatcaaaatgtattcataccttacatactacccaatcaaaggctttacaagttactttcggcacatcgtctgtcaatgccataacttctgcagaaaatgggtcgagtttcggcctttcgcatcctttgcctttctcttgtaaacagtacttcatggcgcggttttcaactatactgtaaagtaattaaagtaattacctacataattgtatgattggactaccttgagtgacttggactaagatcttggactaagatcggaaacctgatcatgtagatcgaatggactctaaatccgttcagccgggttagattcccggggtttccgttttttataatattaataagttatggaagaacggtgtctcccaatacaggctgtctatgcttaacgggaggctccaatcacaatcttttttgtacaatcc

SEQ ID NO: 4 - DNA, 554 bp, Artificial Sequence
  tRNA(Pyl) expression casette
  promoter (1)..(385)
  tRNA (386)..(454): tRNA sequence
  3′UTR (455)..(554): Termination signal
ttatgcgagtgaggttaccggaggttcaattacccccttacactgtgtgtaaaatagataacctttttctcaacctaaactcaaactcaaatcatttattgcattcatgtgtacatttagatgatacataattaggagtatacctagtatacctagtataaacacatgaataatagactagctagagtctagtagtgtctacaccagactatttttagttaacagtagtttaactagatggcgctaaattagttcttttagtaaacggtagatggcgctgtacttaatcgtcatacaaatcatgccgaatgtatgagattctacatcgcgctatcaaagtttttattgtgtttgtgagcggtacaataattttgccatagcaagtggaaacctgatcatgtagatcgaatggactctaaatccgttcagccgggttagattcccggggtttccgttttttgaagagtttcagtttggtatggtttttctattttcaaattggtatgaggaaataagcataatcaaatttaatttcttttgtaaaactttagctt

SEQ ID NO: 5 - DNA, 554 bp, Artificial Sequence
  tRNA(Pyl) expression cassette
  promoter (1)..(385)
  tRNA (386)..(454): tRNA sequeence
  3′UTR (455)..(554): Termination signal
agaaataaaattgaaatattcgatcaagttcaattttatgtctactgagatagttgatatagcatacctaccggtaaatttctacgttaaaaaaaacaaaacagaaaatatgtcattcattattttcggtatttagtagcttttaataaataatttcaacataaaaatatacaaaaagaaattattcatattaatttctaattttcaacttaaagatcccgtacagtttgacaaccattaaattaacttatttcttaaagtttaccaacagatggcgttgtactcaacccacatacaaattgcgtcaaatgtatgggattctacatcgcgctatgaaagttttcattgtgtttgtgagcggtacaataattttgccttagcaagtggaaacctgatcatgtagatcgaatggactctaaatccgttcagccgggttagattcccggggtttccgttttttgaagaaattttaaataaaaaaaattgttttattttatttttttaagtattctctattacataattctatacgtaggtatttgtcattctatgcg

SEQ ID NO: 6 - DNA, 554 bp, Artificial Sequence
  tRNA(Pyl) expression cassette
  promoter (1)..(385)
  tRNA (386)..(454): tRNA Sequence
  3′UTR (455)..(554): Termination signal
ttgaaaatcgggttaaaatatacaatatcaacgacatctatcgttcatattcagaaacggattacgagttaactagcgccatctgttgttgtgtaagtaacaacactgatatacttgtgtggaatagttccgacagaatttgtagatggcgctgtaataaaaatattatttaaaaacatgtatttttcacaattttatatattattgtaagatatttcgtgatattttataataaaaaatacattaatagtaaatattgtaattaaaaaaaggtttcaccttatttcattaaagattttaagaaatataacatgaaactctaaatcgcgatatcaacatttttgttgtttggtgcctaatatacaaaaattcgtgctcgaccaccggaaacctgatcatgtagatcgaatggactctaaatccgttcagccgggttagattcccggggtttccgttttttgaagagtttcagtacgtttataattttattattatttatttatagtaaaaacgtgactaataaacaaagacgattgtttatttgtatgcaattt

SEQ ID NO: 7 - DNA, 554 bp, Artificial Sequence
  tRNA(Pyl) expression cassette
  promoter (1)..(385)
  tRNA (386)..(454): tRNA sequence
  3′UTR (455)..(554): Termination signal
tcggttcaaaatatacaataccaacgacatctgtagttcatattcagaaacgtgtcacgggttaactagcgccatctattgttgtgtaagtaatattgataaaacgatgccatactgtgcggaaaagttccgacagaatttatagatggcgctgtaataaaaatattatttaagaacatgtatttttcaaaattttatatattattgtaagatatttcatgatattttataataaaaaatatgttaatagtaaatattgtaattaaaagtgggtttgaccttatttcattgaaaatttaaagaaatataaaacaaaactctaaatcgcgatatcaacatttttgttgttcggtgcctaatgtactaaaattcgtgctttacaaccggaaacctgatcatgtagatcgaatggactctaaatccgttcagccgggttagattcccggggtttccgttttttgaagaatgtcgctaagatagaattttaataattctttatttttggtaaatccgtgactaaaaacaaaagtgattgtttatttttttaacttaag

SEQ ID NO: 8 - DNA, 554 bp, Artificial Sequence
  tRNA(Pyl) expression cassette
  promoter (1)..(385)
  tRNA (386)..(454): tRNA sequence
  3′UTR (455)..(554): Termination signal
attgtttattttttataaaagctgatatataaataaatattaactgataaataaaaaaatactttcttggaacaattgaagggaataatgatgaaaaattttgctacgtgtaaaaaaaggactttagttcttttacgtttcgttagatggcgctttttacaaagtacgactaccaagtttaattttattcattaaaaatagaaaattagtagaatttgtaaatttattctacaaaaaaatataaataaagtctgaaattttactatacataatttttcaatccaaaatcaattactatcatccagtaatttacaaaatctctgcatcgcgctagtaaaatttttatgctaagaatcatgtataccaaaacggttattccacaagtggaaacctgatcatgtagatcgaatggactctaaatccgttcagccgggttagattcccggggtttccgttttttggacattttcattttggtgaatattttaaaaatgctttgtatttcatcacatcttttattacatttctttcatcacatcacagtgatttttttt

SEQ ID NO: 9 - DNA, 69 bp, Methanosarcina mazeii
  tRNA (1)..(69): tRNA sequence
ggaaacctgatcatgtagatcgaatggactctaaatccgttcagccgggttagattcccggggtttccg

SEQ ID NO: 10 - DNA, 107 bp, Homo sapiens
gtgctcgctccggcagcacatatactaaaattggaacgatacagagaagattagcatggcccctgcgcaaggatgacacgcaaattcgtgaagcgttccatattttt

SEQ ID NO: 11 - DNA, 107 bp, Drosophila melanogaster
gttcttgcttcggcagaacatatactaaaattggaacgatacagagaagattagcatggcccctgcgcaaggatgacacgcaaaatcgtgaagcgttccacattttt

SEQ ID NO: 12 - DNA, 108 bp, Spodoptera frugiperda
gtacttgcttcggcagtacatatactaaaattggaacgatacagagaagattagcatggcccctgcgcaaggatgacacgcaaaatcgtgaagcgttccacatttttt

SEQ ID NO: 13 - DNA, 108 bp, Bombyx mori; identical to SEQ ID NO: 12

SEQ ID NO: 14 - DNA, 74 bp, Bombyx mori
gtacatatactaaaattggaacgatacagagacgattagcatggcccctgcgcaaggatgacacgcaaaatcgt

SEQ ID NO: 15 - DNA, 356 bp, Artificial Sequence
  U6 (H.sapiens) promoter tRNApyl 3term
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaaacctgatcatgtagatcgaatggactctaaatccgttcagccgggttagattcccggggtttccgtttttcggggaaatgtgcgcgga

SEQ ID NO: 16 - DNA, 561 bp, Artificial Sequence
  U6 (D. melanogaster) promoter-tRNA Pyl-3term
gttcgacttgcagcctgaaatacggcacgagtaggaaaagccgagtcaaatgccgaatgcagagtctcattacagcacaatcaactcaagaaaaactcgacacttttttaccatttgcacttaaatccttttttattcgttatgtatactttttttggtccctaaccaaaacaaaaccaaactctcttagtcgtgcctctatatttaaaactatcaatttattatagtcaataaatcgaactgtgttttcaacaaacgaacaatggacactttgattctaaaggaaattttgaaaatcttaagcagagggttcttaagaccatttgccaattcttataattctcaactgtctctttcctgatgttgatcatttatataggtatgttttcctcaatacttcggaaacctgatcatgtagatcgaatggactctaaatccgttcagccgggttagattcccggggtttccgtttttttgctaacctgtgattgctcctactcaaatacaaaaacatcaaattttctgtcaataaagcatatttatttatatttattttacagg

SEQ ID NO: 17 - DNA, 568 bp, Artificial Sequence
  U6  (B.mori) promoter-tRNA Pyl-3term
ttaatattaaataagtacataccttgagaatttaaaaatcgtcaactataagccatacgaatttaagcttggtacttggcttatagataaggacagaataagaattgttaacgtgtaagacaaggtcagatagtcatagtgattttgtcaaagtaataacagatggcgctgtacaaaccataactgttttcatttgtttttatggattttattacaaattctaaaggttttattgttattatttaatttcgttttaattatattatatatctttaatagaatatgttaagagtttttgctctttttgaataatctttgtaaagtcgagtgttgttgtaaatcacgctttcaatagtttagtttttttaggtatatatacaaaatatcgtgctctacaagtggaaacctgatcatgtagatcgaatggactctaaatccgttcagccgggttagattcccggggtttccgtttttttagctttaacactttgaataaatattactctcttaaatatctttgctacaagttgtacttaattgtatttaattgtacaaaatatattaaaat

SEQ ID NO: 18 - DNA, 561 bp, Artificial Sequence; identical to SEQ ID NO: 2
  U6 (S.frugiperda) promoter-tRNA Pyl-3term

SEQ ID NO: 19 - DNA, 2536 bp, Artificial Sequence
  Plasmid pIEx-U6(Human)-tRNAPyl-3′term
cggctcgtatgttgtgtggaattgtgagcggataacaatttcacacaggaaacagctatgaccatgattacgaattcaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaaacctgatcatgtagatcgaatggactctaaatccgttcagccgggttagattcccggggtttccgccatttttcggggaaatgtgcgcggatctagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttc

SEQ ID NO: 20 - DNA, 3160 bp, Artificial Sequence
  Plasmid pIEx-U6(Dm)-2-tRNAPyl-3term
aattcgcgatcgcaaaaagatctgttcgacttgcagcctgaaatacggcacgagtaggaaaagccgagtcaaatgccgaatgcagagtctcattacagcacaatcaactcaagaaaaactcgacacttttttaccatttgcacttaaatccttttttattcgttatgtatactttttttggtccctaaccaaaacaaaaccaaactctcttagtcgtgcctctatatttaaaactatcaatttattatagtcaataaatcgaactgtgttttcaacaaacgaacaatggacactttgattctaaaggaaattttgaaaatcttaagcagagggttcttaagaccatttgccaattcttataattctcaactgtctctttcctgatgttgatcatttatataggtatgttttcctcaatacttcggaaacctgatcatgtagatcgaatggactctaaatccgttcagccgggttagattcccggggtttccgtttttttgctaacctgtgattgctcctactcaaatacaaaaacatcaaattttctgtcaataaagcatatttatttatatttattttacaggggatccacgcgtgcggccgcacagctgtatacacgtgcaagccagccagaactcgccccggaagaccccgaggatctcgagcactaagtgattaacctcaggttatacatatattttgaatttaattaattatacatatattttatattatttttgtcttttattatcgaggggccgttgttggtgtggggttttgcatagaaataacaatgggagttggcgacgttgctgcgccaacaccacctcccttccctcctttcatcatgtatctgtagataaaataaaatattaaacctaaaaacaagaccgcgcctatcaacaaaatgataggcattaacttgccgctgacgctgtcactaacgttggacgatttgccgactaaaccttcatcgcccagtaaccaatctagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacg

SEQ ID NO: 21 - DNA, 4715 bp, Artificial Sequence
  Plasmid pIEx-U6(Dm)-2-tRNAPyl-3′term 4 x
aaataggcgtatcacgaggccctttcgttcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggcgatcgcgttcgacttgcagcctgaaatacggcacgagtaggaaaagccgagtcaaatgccgaatgcagagtctcattacagcacaatcaactcaagaaaaactcgacacttttttaccatttgcacttaaatccttttttattcgttatgtatactttttttggtccctaaccaaaacaaaaccaaactctcttagtcgtgcctctatatttaaaactatcaatttattatagtcaataaatcgaactgtgttttcaacaaacgaacaatggacactttgattctaaaggaaattttgaaaatcttaagcagagggttcttaagaccatttgccaattcttataattctcaactgtctctttcctgatgttgatcatttatataggtatgttttcctcaatacttcggaaacctgatcatgtagatcgaatggactctaaatccgttcagccgggttagattcccggggtttccgtttttttgctaacctgtgattgctcctactcaaatacaaaaacatcaaattttctgtcaataaagcatatttatttatatttattttacagggaattcgttcgacttgcagcctgaaatacggcacgagtaggaaaagccgagtcaaatgccgaatgcagagtctcattacagcacaatcaactcaagaaaaactcgacacttttttaccatttgcacttaaatccttttttattcgttatgtatactttttttggtccctaaccaaaacaaaaccaaactctcttagtcgtgcctctatatttaaaactatcaatttattatagtcaataaatcgaactgtgttttcaacaaacgaacaatggacactttgattctaaaggaaattttgaaaatcttaagcagagggttcttaagaccatttgccaattcttataattctcaactgtctctttcctgatgttgatcatttatataggtatgttttcctcaatacttcggaaacctgatcatgtagatcgaatggactctaaatccgttcagccgggttagattcccggggtttccgtttttttgctaacctgtgattgctcctactcaaatacaaaaacatcaaattttctgtcaataaagcatatttatttatatttattttacaggagatctgttcgacttgcagcctgaaatacggcacgagtaggaaaagccgagtcaaatgccgaatgcagagtctcattacagcacaatcaactcaagaaaaactcgacacttttttaccatttgcacttaaatccttttttattcgttatgtatactttttttggtccctaaccaaaacaaaaccaaactctcttagtcgtgcctctatatttaaaactatcaatttattatagtcaataaatcgaactgtgttttcaacaaacgaacaatggacactttgattctaaaggaaattttgaaaatcttaagcagagggttcttaagaccatttgccaattcttataattctcaactgtctctttcctgatgttgatcatttatataggtatgttttcctcaatacttcggaaacctgatcatgtagatcgaatggactctaaatccgttcagccgggttagattcccggggtttccgtttttttgctaacctgtgattgctcctactcaaatacaaaaacatcaaattttctgtcaataaagcatatttatttatatttattttacaggggatccgttcgacttgcagcctgaaatacggcacgagtaggaaaagccgagtcaaatgccgaatgcagagtctcattacagcacaatcaactcaagaaaaactcgacacttttttaccatttgcacttaaatccttttttattcgttatgtatactttttttggtccctaaccaaaacaaaaccaaactctcttagtcgtgcctctatatttaaaactatcaatttattatagtcaataaatcgaactgtgttttcaacaaacgaacaatggacactttgattctaaaggaaattttgaaaatcttaagcagagggttcttaagaccatttgccaattcttataattctcaactgtctctttcctgatgttgatcatttatataggtatgttttcctcaatacttcggaaacctgatcatgtagatcgaatggactctaaatccgttcagccgggttagattcccggggtttccgtttttttgctaacctgtgattgctcctactcaaatacaaaaacatcaaattttctgtcaataaagcatatttatttatatttattttacagggtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataa

SEQ ID NO: 22 - DNA, 2281 bp, Artificial Sequence
  Plasmid pIDK
gatactagtatacggacctttaattcaacccaacacaatatattatagttaaataagaattattatcaaatcatttgtatattaattaaaatactatactgtaaattacattttatttacaatcactcgacgaagacttgatcacccgggatctcgagccatggtgctagcagctgatgcatagcatgcggtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagccactacgggtacgtttaaacccatgtgcctggcagataacttcgtataatgtatgctatacgaagttatggtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttcagtgacacaggaacacttaacggctgacagaattagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggatctatgtcgggtgcggagaaagaggtaatgaaatggcacctaggtatc

SEQ ID NO: 23 - DNA, 2700 bp, Artificial Sequence
  Plasmid pIDK-U6(Bm)-2-tRNAPyl-3′term
ggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggatctatgtcgggtgcggagaaagaggtaatgaaatggcacctaggtatcgatactagtttaatattaaataagtacataccttgagaatttaaaaatcgtcaactataagccatacgaatttaagcttggtacttggcttatagataaggacagaataagaattgttaacgtgtaagacaaggtcagatagtcatagtgattttgtcaaagtaataacagatggcgctgtacaaaccataactgttttcatttgtttttatggattttattacaaattctaaaggttttattgttattatttaatttcgttttaattatattatatatctttaatagaatatgttaagagtttttgctctttttgaataatctttgtaaagtcgagtgttgttgtaaatcacgctttcaatagtttagtttttttaggtatatatacaaaatatcgtgctctacaagtggaaacctgatcatgtagatcgaatggactctaaatccgttcagccgggttagattcccggggtttccgtttttttagctttaacactttgaataaatattactctcttaaatatctttgctacaagttgtacttaattgtatttaattgtacaaaatatattaaaatccatggtgctagcagctgatgcatagcatgcggtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagccactacgggtacgtttaaacccatgtgcctggcagataacttcgtataatgtatgctatacgaagttatggtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttcagtgacacaggaacacttaacggctgacagaattagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaacc

SEQ ID NO: 24 - DNA, 3130 bp, Artificial Sequence
  Plasmid pIEx-U6(Sf21)-2-tRNAPyl-3′term
aatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcacttaactactcaaaaagtgagggccagcagctcgaccaatgtaaaaccttgcgaggtgcgaggttaccggggacccaatcaaagagtataataactatagggaaaggcccaaccccccccccccccactgtatgtaaaaatataagacctatttctcaacctataaacctatgcaataaaacatccactagattagtctagtgactagactagaccattgttagttaacagtagttcggctagatggcgccaaattggttcttttagtgaacggtagatggcgctgtactcaatcttcatacaaatcatgttaaatgtatgggattctacatcgcgctatcaaagttttcattgtgtttgtgaagggtacaataattttgccttggcaagtggaaacctgatcatgtagatcgaatggactctaaatccgttcagccgggttagattcccggggtttccgttttttgaagagtttcagtttggtatggtttttctattttcaaattggtatgagggagtaagcataatcaaatttaatttcttttgttaaactttagcttgcggccgcacagctgtatacacgtgcaagccagccagaactcgccccggaagaccccgaggatctcgagcactaagtgattaacctcaggttatacatatattttgaatttaattaattatacatatattttatattatttttgtcttttattatcgaggggccgttgttggtgtggggttttgcatagaaataacaatgggagttggcgacgttgctgcgccaacaccacctcccttccctcctttcatcatgtatctgtagataaaataaaatattaaacctaaaaacaagaccgcgcctatcaacaaaatgataggcattaacttgccgctgacgctgtcactaacgttggacgatttgccgactaaaccttcatcgcccagtaaccaatctagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcatt

SEQ ID NO: 25 - DNA, 6145 bp, Artificial Sequence
  Plasmid pIZT-PylRS-mCherry-GFP(Y39TAG)
aacttcatttttaatttaaaaggatctaggtgaagatcctttttgataatctcatgatgataaacaatgtatggtgctaatgttgcttcaacaacaattctgttgaactgtgttttcatgtttgccaacaagcacctttatactcggtggcctccccaccaccaacttttttgcactgcaaaaaaacacgcttttgcacgcgggcccatacatagtacaaactctacgtttcgtagactattttacataaatagtctacaccgttgtatacgctccaaatacactaccacacattgaacctttttgcagtgcaaaaaagtacgtgtcggcagtcacgtaggccggccttatcgggtcgcgtcctgtcacgtacgaatcacattatcggaccggacgagtgttgtcttatcgtgacaggacgccagcttcctgtgttgctaaccgcagccggacgcaactccttatcggaacaggacgcgcctccatatcagccgcgcgttatctcatgcgcgtgaccggacacgaggcgcccgtcccgcttatcgcgcctataaatacagcccgcaacgatctggtaaacacagttgaacagcatctgttcgaatttaaagcttggtaccatggacaaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctggcaccaaatctgtataactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccaattccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaatctagagggcccgcggttcgaaggtaagcctatccctaaccctctcctcggtctcgattctacgcgtaccggtcatcatcaccatcaccattgagtttatctgactaaatcttagtttgtattgtcatgttttaatacaatatgttatgtttaaatatgtttttaataaattttataaaataatttcaacttttattgtaacaacattgtccatttacacactcctttcaagcgcgtgggatcgatgctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcgaaacacgcacggcgcgcgcacgcagcttagcacaaacgcgtcgttgcacgcgcccaccgctaaccgcaggccaatcggtcggccggcctcatatccgctcaccagccgcgtcctatcgggcgcggcttccgcgcccattttgaataaataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgccgggatctgccgggctgcagcacgtgttgacaattaatcatcggcatagtatatcggcatagtataatacgacaaggtgaggaactaaaccatggacggccgcctggaaagcaccccgccgaaaaaaaaacgcaaagtggaagatagcgcgagcgattacaaagatgatgatgataaagtggcgtacgcggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcacctccgacggccccgtaatgcagaagaagacgatgggctgggaggcctcctccgagcggatgtacaccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaccctgcaggaattcccccctcccccagcgagcgattacaaagatgatgatgataaagtgagcaagggcgaggagctgttcaccggggtggtgcccatcctggtcgagctggacggcgacgtaaacggccacaagttcagcgtgtccggcgagggcgagggcgatgccacctatggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcatcaccatcaccatcactgaccgacgccgaccaacaccgccggtccgacgcggcccgacgggtccgaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtctggatcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaa

SEQ ID NO: 26 - DNA, 1365 bp, Methanosarcina mazeii
  CDS (1)..(1365)
atggacaaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctggcaccaaatctgtataactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccaattccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 27 - protein, 454 aa, Methanosarcina mazeii
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 28 - DNA, 6145 bp, Artificial Sequence; identical to SEQ ID NO: 25
  Plasmid pIZT-PylRS-mCherry-GFP(WT)

SEQ ID NO: 29 - DNA, 1365 bp, Artificial Sequence
  Mutant PylRS AF
  CDS (1)..(1365)
atggacaaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctggcaccaaatctggctaactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtttggcgacaccctggatgtcatgcacggcgacctggaactgtctagtgccgttgttggaccaattccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 30 - protein, 454 aa, Artificial Sequence
  Synthetic Construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLANYLRKLDRALPDPIKIFEIGPCYRKESDGKEHLEEFTMLNFCQMGSGCTRENLESIITDFLNHLGIDFKIVGDSCMVFGDTLDVMHGDLELSSAVVGPIPLDREWGIDKPWIGAGFGLERLLKVKHDFKNIKRAARSESYYNGISTNL

SEQ ID NO: 31 - DNA, 4733 bp, Artificial Sequence
  Plasmid pBAD-GFP(Y39TAG)-6His
aaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggattacaaagatgatgatgataaagtgagcaagggcgaggagctgttcaccggggtggtgcccatcctggtcgagctggacggcgacgtaaacggccacaagttcagcgtgtccggcgagggcgagggcgatgccacctagggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcatcaccatcaccatcactgagcggccgcactcgagagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgct

SEQ ID NO: 32 - DNA, 3005 bp, Artificial Sequence
  Plasmid pUCDM
aattctgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttatggtcaaacagagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactagcataaccccttggggcctctaaacgggtcaacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgacattcatccggggtcagcaccgtttctgcggactggctttctacgtgttccgcttcctttagcagcccttgcgccctgagtgcttgcggcagcgtgaagct

SEQ ID NO: 33 - DNA, 4717 bp, Artificial Sequence
  Plasmid pUCDM-PylRSWT-U6(Sf21)-2-tRNAPyl-3′term
aaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatttacaggttcgtagagatcccgttataatacgattcagaacgggcggcacgtttgatgtttttgaagtcgtgtttcactttcagcaggcgttccagaccgaatcctgctccgatccacggtttgtcgataccccactcacggtccagcggaattggtccaacaacggcactagacagttccaggtcgccgtgcatgacatccagggtgtcgccatacaccatacagctgtcgcccacaattttgaagtcaatgcccaggtggttcagaaaatcggtgatgatgctttccaggttctcacgagtacaacctgaacccatttggcaaaagttcagcatggtaaactcctccagatgttctttaccgtcggactctttacgataacacgggccgatctcgaagattttgataggatcaggcagggcacggtccagtttgcgcagatagttatacagatttggtgccagcatagggcgcagacagaagtttttatccacacggaaaatttgtttgctcagttcggtatcattgtcgatgcccatacgctcgatatactccagaggaatcagaatcggggatttgatctccagaaagccacgatccacgaaaaagcgggtgatttcacgttccagtttccccagatagttctcacgttcttcggcatagatttgttgcaggtcttttttacgacgtgacagcagttcgctctccagttcacgaaacggtttgccggaattcaggctgatttcgtctttcggattcagcagaacctccagacgatcggtttgggattttgtcagtgctggagctgatgcttgaaccggggcagacatgcttgtaatcggattggtattgcctttaaccagggcgctagcggtggcaccggtgctaatactgctaatgctggtgctcacacttgctggaacagaaacggactcctgggtagaaacaggaatggccggagagaatttgcttccagacggctgtgcctgtgctgcttcagtgttttccagtggtttaggggcacgagcaacggatttcggcattgcttttttagtacgggtaggagcgctaacgactttcactttcacgcttgtttggtcctcattggcttttgtcaggaatttgttcagatcctcatcggacacacggcaacgtttacaggttttacgatatttgtggtgacgcagtgcacgtgctgtacgagaagagcggctattgttcacaaccagatgatcgccacacgccatctcaatatagattttcgaacggctaacctcgtggtgtttgattttatgaatggttccggtacgactcatccacagaccagtagcagagatcagggtattcagcggttttttgtccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagacttaactactcaaaaagtgagggccagcagctcgaccaatgtaaaaccttgcgaggtgcgaggttaccggggacccaatcaaagagtataataactatagggaaaggcccaaccccccccccccccactgtatgtaaaaatataagacctatttctcaacctataaacctatgcaataaaacatccactagattagtctagtgactagactagaccattgttagttaacagtagttcggctagatggcgccaaattggttcttttagtgaacggtagatggcgctgtactcaatcttcatacaaatcatgttaaatgtatgggattctacatcgcgctatcaaagttttcattgtgtttgtgaagggtacaataattttgccttggcaagtggaaacctgatcatgtagatcgaatggactctaaatccgttcagccgggttagattcccggggtttccgttttttgaagagtttcagtttggtatggtttttctattttcaaattggtatgagggagtaagcataatcaaatttaatttcttttgttaaactttagctttctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactagcataaccccttggggcctctaaacgggtcaacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttatttgtcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccg

SEQ ID NO: 34 - DNA, 4713 bp, Artificial Sequence
  Plasmid pUCDM-PylRSAF-U6(Sf21)-2-tRNAPyl-3′term
tcgagatggacaaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgcttgcaccaaatctggctaactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtttggcgacaccctggatgtcatgcacggcgacctggaactgtctagtgccgttgttggaccaattccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaaatgcatagcatgcggtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagccactacgggtacgtttaaaccagataacttcgtataatgtatgctatacgaagttatggtaccgcggccgcgtagaggatctgttgatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctctgtttgaccataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttcagtgacacaggaacacttaacggctgacagaattagcttcacgctgccgcaagcactcagggcgcaagggctgctaaaggaagcggaacacgtagaaagccagtccgcagaaacggtgctgaccccggatgaatgtcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagatccggatatagttcctcctttcagcaaaaaacccctcaagacccgtttagaggcccctaggggttgacccgtttagaggccccaaggggttatgctagttattgctcagcggtggcagcagccaactcagcttcctttcgggctttgttagcagccggatcttctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagcttgtcgagactgcaggctctagaaagctaaagtttaacaaaagaaattaaatttgattatgcttactccctcataccaatttgaaaatagaaaaaccataccaaactgaaactcttcaaaaaacggaaaccccgggaatctaacccggctgaacggatttagagtccattcgatctacatgatcaggtttccacttgccaaggcaaaattattgtacccttcacaaacacaatgaaaactttgatagcgcgatgtagaatcccatacatttaacatgatttgtatgaagattgagtacagcgccatctaccgttcactaaaagaaccaatttggcgccatctagccgaactactgttaactaacaatggtctagtctagtcactagactaatctagtggatgttttattgcataggtttataggttgagaaataggtcttatatttttacatacagtggggggggggggggttgggcctttccctatagttattatactctttgattgggtccccggtaacctcgcacctcgcaaggttttacattggtcgagctgctggccctcactttttgagtagttaagtctattaatattccggagtaggtcgcgaatcgatactagtatacggacctttaattcaacccaacacaatatattatagttaaataagaattattatcaaatcatttgtatattaattaaaatactatactgtaaattacattttatttacaatcactcgacgaagacttgatcacccgggatc

SEQ ID NO: 35 - DNA, 4412 bp, Artificial Sequence
  Plasmid pACEBac-DUAL
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtacgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 36 - DNA, 5100 bp, Artificial Sequence
  Plasmid pACEBac-DUAL-GFP(Y39TAG)-6His
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtacgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccatggattacaaagatgatgatgataaagtgagcaagggcgaggagctgttcaccggggtggtgcccatcctggtcgagctggacggcgacgtaaacggccacaagttcagcgtgtccggcgagggcgagggcgatgccacctagggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcatcaccatcaccatcactgactgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 37 - DNA, 717 bp, Artificial Sequence
  CDS of Herceptin heavy chain- 6His
  CDS (1)..(717)
atggaagtgcagctggtcgagtccggtggtggcctggttcagcctggtggttccctgcgtctgtcctgcgctgcttccggtttcaacatcaaggacacctacatccactgggtccgccaggctcccggcaagggattggaatgggtggcccgtatctaccccaccaacggttacacccgttacgctgactccgtgaagggccgtttcaccatctccgctgacacctccaagaacaccgcttacctgcagatgaactccctgcgtgctgaggacaccgctgtgtactactgctcccgttggggtggcgacggtttctacgctatggactactggggccagggcaccctggtcaccgtgtcctctgcttccaccaagggcccctccgtgttccctctggctccatcctccaagtccacctccggtggaaccgctgctctgggttgcctggtcaaggactacttccccgagcccgtgaccgtgtcttggaactccggtgctctgacctccggcgtgcacaccttccctgctgtcctgcagtcctccggcctgtactccctgtcctccgtcgtgactgtgccctcatcctccctgggcacccagacctacatctgcaacgtgaaccacaagccctccaacaccaaggtggacaagaaggtcgagccccccaagtcctgcgacaagacccacacttgcccccctcatcaccatcaccaccac

SEQ ID NO: 38 - protein, 239 aa, Artificial Sequence
  Synthetic Construct
MEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPHHHHHH

SEQ ID NO: 39 - DNA, 648 bp, Artificial Sequence
  CDS Herceptin light chain
  CDS (1)..(648)
atggacatccagatgacccagtccccctcctccctgtccgcttccgtgggagatcgtgtgaccatcacttgccgtgcttcccaggacgtgaacaccgctgtggcttggtaccagcagaagcccggcaaggctcccaagctgctgatctactccgctagcttcctgtactccggtgtcccctcccgtttctccggttcccgttccggtactgacttcaccctgaccatctccagcctgcagcccgaggacttcgctacctactactgccagcagcactacaccaccccccccaccttcggccagggtactaaggtcgagatcaagcgtaccgtggctgctccctccgtgttcatcttcccaccctccgacgagcagctgaagtccggcactgcttccgtcgtgtgcctgctgaacaacttctacccccgcgaggctaaggtgcagtggaaggtggacaacgctctgcagtccggcaactcccaagagtccgtgaccgagcaggactccaaggactctacctactctctgtcctctaccctgaccctgtccaaggctgactacgagaagcacaaggtgtacgcttgcgaagtgacccaccagggcctgtcctccccagtgaccaagtccttcaaccgtggcgagtgctaa

SEQ ID NO: 40 - protein, 215 aa, Artificial Sequence
  Synthetic Construct
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 5747 bp, Artificial Sequence
  Plasmid pAceBac-DUAL-Herceptin-6His
gatccatggacatccagatgacccagtccccctcctccctgtccgcttccgtgggagatcgtgtgaccatcacttgccgtgcttcccaggacgtgaacaccgctgtggcttggtaccagcagaagcccggcaaggctcccaagctgctgatctactccgctagcttcctgtactccggtgtcccctcccgtttctccggttcccgttccggtactgacttcaccctgaccatctccagcctgcagcccgaggacttcgctacctactactgccagcagcactacaccaccccccccaccttcggccagggtactaaggtcgagatcaagcgtaccgtggctgctccctccgtgttcatcttcccaccctccgacgagcagctgaagtccggcactgcttccgtcgtgtgcctgctgaacaacttctacccccgcgaggctaaggtgcagtggaaggtggacaacgctctgcagtccggcaactcccaagagtccgtgaccgagcaggactccaaggactctacctactctctgtcctctaccctgaccctgtccaaggctgactacgagaagcacaaggtgtacgcttgcgaagtgacccaccagggcctgtcctccccagtgaccaagtccttcaaccgtggcgagtgctaagaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagtggtggtgatggtgatgaggggggcaagtgtgggtcttgtcgcaggacttggggggctcgaccttcttgtccaccttggtgttggagggcttgtggttcacgttgcagatgtaggtctgggtgcccagggaggatgagggcacagtcacgacggaggacagggagtacaggccggaggactgcaggacagcagggaaggtgtgcacgccggaggtcagagcaccggagttccaagacacggtcacgggctcggggaagtagtccttgaccaggcaacccagagcagcggttccaccggaggtggacttggaggatggagccagagggaacacggaggggcccttggtggaagcagaggacacggtgaccagggtgccctggccccagtagtccatagcgtagaaaccgtcgccaccccaacgggagcagtagtacacagcggtgtcctcagcacgcagggagttcatctgcaggtaagcggtgttcttggaggtgtcagcggagatggtgaaacggcccttcacggagtcagcgtaacgggtgtaaccgttggtggggtagatacgggccacccattccaatcccttgccgggagcctggcggacccagtggatgtaggtgtccttgatgttgaaaccggaagcagcgcaggacagacgcagggaaccaccaggctgaaccaggccaccaccggactcgaccagctgcacttccatcccgggtgatcaagtcttcgtcgagtgattgtaaataaaatgtaatttacagtatagtattttaattaatatacaaatgatttgataataattcttatttaactataatatattgtgttgggttgaattaaaggtccgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcg

SEQ ID NO: 42 - DNA, 6349 bp, Artificial Sequence
  Plasmid pFastBac-Dual-6HisTAF11/TAF13
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagaacaattgcggccgcggatcctcaagatccataatttgcttcatcaaatgcttttctagctcgtttcaattcttcattcatagtaagcaagtctttaaccctggcaaacttccttgggtcctttcgaatcaagaagacgatatcttcaacttgtactcgaccttgtcttccaattgacattgccttgtgagtcatttcagtgataaactctatgacaagatcttcaagaatatccactgactcagtataaggattctggtcatccccaaagccatacatcatacatcgcaattctttagaaaaaagtctctttcttttaccctgtccaccttctgcacctcctccaatttcttcattttcttcctcaaacgtggggtcttcttcctcatctgccatatgttctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtactccggaatattaatagatcatggagataattaaaatgataaccatctcgcaaataaataagtattttactgttttcgtaacagttttgtaataaaaaaacctataaatattccggattattcataccgtcccaccatcgggcgcggatctcggtccgaaaccatgtcgtactaccatcaccatcaccatcacgattacgatatcccaacgaccgaaaacctgtattttcagggcgccatggacgatgcccacgagtcgccctccgacaaaggtggagagacaggggagtcggatgagacggccgctgtgcccggggacccgggggctaccgacaccgatggaatcccagaggaaactgacggagacgcagatgtggacttgaaagaagctgcagcggaggaaggcgagctcgagagtcaggatgtctcagatttaacaacagttgaaagggaagactcatcattacttaatcctgcagccaaaaaactgaaaatagataccaaagaaaagaaagagaaaaagcagaaagtagatgaagatgagattcagaagatgcaaatcctggtttcttctttttctgaggagcagctgaaccgttatgaaatgtatcgccgctcagctttccctaaggcagccatcaaaaggctgatccagtccatcactggcacctctgtgtctcagaatgttgttattgctatgtctggtatttccaaggttttcgtcggggaggtggtagaagaagcactggatgtgtgtgagaagtggggagaaatgccaccactacaacccaaacatatgagggaagccgttagaaggttaaagtcaaaaggacagatccctaactcgaagcacaaaaaaatcatcttcttctaaggatccggaattcaaaggcctacgtcgacgagctcaactagtgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatggg

SEQ ID NO: 43 - DNA, 711 bp, Artificial Sequence
  CDS 6His-TAF11
  CDS (1)..(711)
atgtcgtactaccatcaccatcaccatcacgattacgatatcccaacgaccgaaaacctgtattttcagggcgccatggacgatgcccacgagtcgccctccgacaaaggtggagagacaggggagtcggatgagacggccgctgtgcccggggacccgggggctaccgacaccgatggaatcccagaggaaactgacggagacgcagatgtggacttgaaagaagctgcagcggaggaaggcgagctcgagagtcaggatgtctcagatttaacaacagttgaaagggaagactcatcattacttaatcctgcagccaaaaaactgaaaatagataccaaagaaaagaaagagaaaaagcagaaagtagatgaagatgagattcagaagatgcaaatcctggtttcttctttttctgaggagcagctgaaccgttatgaaatgtatcgccgctcagctttccctaaggcagccatcaaaaggctgatccagtccatcactggcacctctgtgtctcagaatgttgttattgctatgtctggtatttccaaggttttcgtcggggaggtggtagaagaagcactggatgtgtgtgagaagtggggagaaatgccaccactacaacccaaacatatgagggaagccgttagaaggttaaagtcaaaaggacagatccctaactcgaagcacaaaaaaatcatcttcttctaa

SEQ ID NO: 44 - protein, 236 aa, Artificial Sequence
  Synthetic Construct
MSYYHHHHHHDYDIPTTENLYFQGAMDDAHESPSDKGGETGESDETAAVPGDPGATDTDGIPEETDGDADVDLKEAAAEEGELESQDVSDLTTVEREDSSLLNPAAKKLKIDTKEKKEKKQKVDEDEIQKMQILVSSFSEEQLNRYEMYRRSAFPKAAIKRLIQSITGTSVSQNVVIAMSGISKVFVGEVVEEALDVCEKWGEMPPLQPKHMREAVRRLKSKGQIPNSKHKKIIFF

SEQ ID NO: 45 - DNA, 375 bp, Artificial Sequence
  CDS TAF13
  CDS (1)..(375)
atggcagatgaggaagaagaccccacgtttgaggaagaaaatgaagaaattggaggaggtgcagaaggtggacagggtaaaagaaagagacttttttctaaagaattgcgatgtatgatgtatggctttggggatgaccagaatccttatactgagtcagtggatattcttgaagatcttgtcatagagtttatcactgaaatgactcacaaggcaatgtcaattggaagacaaggtcgagtacaagttgaagatatcgtcttcttgattcgaaaggacccaaggaagtttgccagggttaaagacttgcttactatgaatgaagaattgaaacgagctagaaaagcatttgatgaagcaaattatggatcttga

SEQ ID NO: 46 - protein, 124 aa, Artificial Sequence
  Synthetic Construct
MADEEEDPTFEEENEEIGGGAEGGQGKRKRLFSKELRCMMYGFGDDQNPYTESVDILEDLVIEFITEMTHKAMSIGRQGRVQVEDIVFLIRKDPRKFARVKDLLTMNEELKRARKAFDEANYGS

SEQ ID NO: 47 - DNA, 609 bp, Artificial Sequence
  CDS 6His-TBP
  CDS (1)..(609)
atgggcagcagccatcatcatcatcatcacagcagcggcctggtgccgcgcggcagccatatgtctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaacatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaa

SEQ ID NO: 48 - protein, 202 aa, Artificial Sequence
  Synthetic Construct
MGSSHHHHHHSSGLVPRGSHMSGIVPQLQNIVSTVNLGCKLDLKTIALRARNAEYNPKRFAAVIMRIREPRTTALIFSSGKMVCTGAKSEEQSRLAARKYARVVQKLGFPAKFLDFKIQNMVGSCDVKFPIRLEGLVLTHQQFSSYEPELFPGLIYRMIKPRIVLLIFVSGKVVLTGAKVRAEIYEAFENIYPILKGFRKTT

SEQ ID NO: 49 - DNA, 5345 bp, Artificial Sequence
  Plasmid pBAD-TBP-Int-CBD-12His
catggcctctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaacatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacggcctgcatcacgggagatgcactagttgccctacccgagggcgagtcggtacgcatcgccgacatcgtgccgggtgcgcggcccaacagtgacaacgccatcgacctgaaagtccttgaccggcatggcaatcccgtgctcgccgaccggctgttccactccggcgagcatccggtgtacacggtgcgtacggtcgaaggtctgcgtgtgacgggcaccgcgaaccacccgttgttgtgtttggtcgacgtcgccggggtgccgaccctgctgtggaagctgatcgacgaaatcaagccgggcgattacgcggtgattcaacgcagcgcattcagcgtcgactgtgcaggttttgcccgcggaaaacccgaatttgcgcccacaacctacacagtcggcgtccctggactggtgcgtttcttggaagcacaccaccgagacccggacgcccaagctatcgccgacgagctgaccgacgggcggttctactacgcgaaagtcgccagtgtcaccgacgccggcgtgcagccggtgtatagccttcgtgtcgacacggcagaccacgcgtttatcacgaacgggttcgtcagccacgctactggcctcaccggtctgaactcaggcctcacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaagcgcatcatcatcatcatcatcatcaccatcaccatcactgagcggccgcactcgagagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaac

SEQ ID NO: 50 - DNA, 4164 bp, Artificial Sequence
  Plasmid pIEx-ccdb
cgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgaccatggcacaccaccatcaccaccatcaccaccatcactcttctggtctggaagttctgttccaggggcccatggactaatgaggtaccggatccgaattcgagctccgtcgacaagcttgatatcgaattcctgcagcccaaactgcagttgacaacataaaaactttgtgttatacttgtaacgtaaggaggtaatgattcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataagcttgcggccgcacagctgtatacacgtgcaagccagccagaactcgccccggaagaccccgaggatctcgagcactaagtgattaacctcaggttatacatatattttgaatttaattaattatacatatattttatattatttttgtcttttattatcgaggggccgttgttggtgtggggttttgcatagaaataacaatgggagttggcgacgttgctgcgccaacaccacctcccttccctcctttcatcatgtatctgtagataaaataaaatattaaacctaaaaacaagaccgcgcctatcaacaaaatgataggcattaacttgccgctgacgctgtcactaacgttggacgatttgccgactaaaccttcatcgcccagtaaccaatctagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcccggg